(19) United States
(12) Patent Application Publication
(10) Pub. No.: US 2024/0399001 A1
(43) Pub. Date: Dec. 5, 2024

(54) RADIOTRACERS AND THERAPEUTICS BINDING TO FIBROBLAST ACTIVATION PROTEIN (FAP)

(71) Applicant: BLUE EARTH DIAGNOSTICS LIMITED, Oxford Oxfordshire (GB)

(72) Inventors: Sabitur RAHMAN, Oxford Oxfordshire (GB); Romain BEJOT, Oxford Oxfordshire (GB); Guyan LIANG, Monore Township, NJ (US); Abdul Karim HAJI, Oxford Oxfordshire (GB); Colin BARNES, Cambridge (GB)

(73) Assignee: BLUE EARTH DIAGNOSTICS LIMITED, Oxford Oxfordshire (GB)

(21) Appl. No.: 18/577,580

(22) PCT Filed: Jul. 8, 2022
(86) PCT No.: PCT/US2022/073544

§ 371 Date: Jan. 8, 2024

(30) Foreign Application Priority Data
Jul. 9, 2021 (GB) 2109922.1

(51) Int. Cl. A61K 51/04; A61K 51/08

(52) U.S. Cl. CPC A61K 51/0455; A61K 51/0446; A61K 51/0472; A61K 51/088

(57) ABSTRACT

The present invention relates to a ligand-SIFA conjugate, comprising, within in a single molecule two separate moieties: (a) one or more ligands which are capable of binding to Fibroblast Activation Protein (FAP), and (b) a silicon-fluoride acceptor (SI FA) moiety which comprises a covalent bond between a silicon atom and a fluorine atom.

### FIELD OF THE INVENTION

The present invention relates to therapeutic and/or diagnostic useful compounds, in particular compounds which are useful in a variety of therapeutic and/or diagnostic areas associated with elevated FAP expression, including the treatment and/or diagnosis of various cancers. Suitably, the present invention relates to a ligand-SIFA conjugate (i.e. a compound) comprising within in a single molecule: (a) one or more ligands which are capable of binding to Fibroblast Activation Protein (FAP), and (b) a silicon-fluoride acceptor (SIFA) moiety which comprises a covalent bond between a silicon and a fluorine atom and which can be labeled with ¹⁸F by isotopic exchange of ¹⁹F by ¹⁸F or which is labeled with ¹⁸F.

### BACKGROUND OF THE INVENTION

### Fibroblast Activation Protein (FAP)

Fibroblast activation protein (FAP) is best known for its heightened expression in tumour stroma. This atypical serine protease has both dipeptidyl peptidase and endopeptidase activities, cleaving substrates at a post-proline bond. FAP expression is difficult to detect in non-diseased adult organs, but is greatly upregulated in sites of tissue remodelling, which include liver fibrosis, lung fibrosis, atherosclerosis, arthritis, tumours and embryonic tissues. FAP is thought to be involved in the control of fibroblast growth or epithelial-mesenchymal interactions during development, tissue repair, and epithelial carcinogenesis. FAP expression is seen on activated stromal fibroblasts of more than 90% of all human carcinomas. Stromal fibroblasts play an important role in the development, growth and metastasis of carcinomas. Due to its restricted expression pattern and dual enzymatic activities, FAP is emerging as a unique therapeutic target and several approaches of FAP targeting mainly in cancer treatment are currently being tested (Rui L. et al Cancer Biology & Therapy, 2012, 13:3, 123-129).

### ¹⁸F Labelling

¹⁸F labelling is a well-known radiolabeling technique and has been used for example for positron emission tomography (PET) imaging in conjugates that target prostate specific membrane antigen (PSMA). An attractive approach for introducing ¹⁸F labels is the use of silicon fluoride acceptors (SIFA). Silicon fluoride acceptors are described, for example, in Lindner et al., Bioconjugate Chemistry 25, 738-749 (2014). In order to preserve the silicon-fluoride bond, the use of silicon fluoride acceptors introduces the necessity of sterically demanding groups around the silicone atom. This in turn renders silicon fluoride acceptors highly hydrophobic. In terms of binding to PSMA, the hydrophobic moiety provided by the silicone fluoride acceptor may be exploited for the purpose of establishing interactions of the radio-diagnostic or -therapeutic compound with the hydrophobic pocket of PSMA described in Zhang et al., Journal of the American Chemical Society 132, 12711-12716 (2010). Yet, prior to binding, the higher degree of lipophilicity introduced into the molecule poses a severe problem with respect to the development of radiopharmaceuticals with suitable in vivo biodistribution, i.e. low unspecific binding in non-target tissue.

WO2019/020831 and WO2020/157184 disclose ligand-SIFA-chelator conjugates. WO2019/083990, WO2019/154886, WO2018/111989, WO2021/005131 and WO2021/005125 disclose compounds comprising FAP ligands.

There exists a need for imaging agents that can identify the presence of diseases associated with elevated FAP expression in human tissue. Such diseases may include cancer, chronic inflammation, atherosclerosis, fibrosis, tissue remodelling and keloid disorder.

The present invention seeks to provide FAP-targeted radio-diagnostics and/or radio-therapeutics which contain a silicon-fluorine containing moiety and which are characterized by favourable in-vivo properties.

Further, the present invention seeks to provide improved radio-therapeutics and/or radio-diagnostics for medical indications associated with elevated FAP expression.

Further, the present invention seeks to provide FAP-targeted and PSMA-targeted combination radio-diagnostics and/or radio-therapeutics which contain a silicon-fluorine containing moiety and which are characterized by favourable in-vivo properties.

Further, the present invention seeks to provide improved radio-therapeutics and/or radio-diagnostics for medical indications associated with elevated FAP expression and elevated prostate-specific membrane antigen (PSMA) expression.

### SUMMARY OF THE INVENTION

According to a first aspect of the invention there is provided a ligand-SIFA conjugate, comprising, within a single molecule, two separate moieties:
(a) one or more ligand(s) which is capable of binding to Fibroblast Activation Protein (FAP); and,
(b) a silicon-fluoride acceptor (SIFA) moiety which comprises a covalent bond between a silicon and a fluorine atom and which SIFA is optionally labelled with ¹⁸F:
or a pharmaceutically or diagnostically acceptable salt or solvate thereof;
which conjugates are referred together herein as “the conjugates of the invention” or “conjugates of some embodiments of the invention”, and which conjugates may also optionally comprise additional moieties.

Suitably, the conjugates of the invention comprise a single chemical entity comprising within a single molecule both (a) said one or more FAP ligand(s) and (b) said SIFA moiety.

Suitably, the SIFA moiety in the conjugates of the invention may be optionally radiolabeled with an ¹⁸F label. The ¹⁸F radiolabel may be introduced into the SIFA moiety by isotopic ¹⁹F-¹⁸F exchange of the SIFA moiety by techniques well known to those skilled in the art, for example as disclosed in PCT/EP2020/052268. Preferably, the conjugates of the invention include a SIFA moiety which is radiolabeled with ¹⁸F. Suitably, the inclusion of ¹⁸F radiolabel in the SIFA moiety allows the conjugates of the invention to be used as a radio-diagnostic tracer, for example in PET imaging.

The term “pharmaceutically or diagnostically acceptable salt or solvate” includes salts and solvates as described herein.

In some embodiments, the conjugates of the invention may further comprise (c) one or more chelating moieties (CM). Said one or more chelating moieties (CM), when present, may optionally contain a chelated nonradioactive cation or radioactive cation. Preferred conjugates of the invention further comprise said (c) one or more chelating moieties (CM) containing a chelated nonradioactive cation or radioactive cation as identified herein.

In some embodiments, the conjugates of the invention may further comprise (d) one or more ligands which are capable of binding to prostate-specific membrane antigen (PSMA).

Suitably, moieties (a) and (b), and (c) and (d) when present, each represent a separate moiety within the single molecule of the conjugates of the invention.

Accordingly, the conjugates of the invention include ligand-SIFA conjugates comprising, within a single molecule, two separate moieties (a) and (b), wherein: (a) is one or more ligands which are capable of binding to Fibroblast Activation Protein (FAP); and, (b) is a silicon-fluoride acceptor (SIFA) moiety which comprises a covalent bond between a silicon and a fluorine atom and which is optionally labelled with ¹⁸F; and, wherein said ligand-SIFA conjugate optionally includes within said single molecule:
(c) one or more chelating moieties (CM), optionally containing a chelated nonradioactive cation or radioactive cation; or,
(d) one or more ligands which are capable of binding to prostate-specific membrane antigen (PSMA); or,
(e) a combination of both (c) said one or more chelating moieties (CM) and (d) said one or more PSMA ligands;
or a pharmaceutically or diagnostically acceptable salt or solvate thereof.

Thus, the conjugates of the invention include:

Ligand-SIFA conjugates comprising, within a single molecule, two separate moieties:
(a) one or more ligand(s) which is capable of binding to Fibroblast Activation Protein (FAP); and,
(b) a silicon-fluoride acceptor (SIFA) moiety which comprises a covalent bond between a silicon and a fluorine atom and which is optionally labelled with ¹⁸F;
or a pharmaceutically or diagnostically acceptable salt or solvate thereof.

Ligand-SIFA conjugates comprising, within a single molecule, three separate moieties:
(a) one or more ligands which are capable of binding to Fibroblast Activation Protein (FAP);
(b) a silicon-fluoride acceptor (SIFA) moiety which comprises a covalent bond between a silicon and a fluorine atom and which is optionally labelled with ¹⁸F; and
(c) one or more chelating moieties (CM), optionally containing a chelated nonradioactive or radioactive cation;
or a pharmaceutically or diagnostically acceptable salt or solvate thereof.

Ligand-SIFA conjugates comprising, within a single molecule, three separate moieties:
(a) one or more ligands which are capable of binding to Fibroblast Activation Protein (FAP);
(b) a silicon-fluoride acceptor (SIFA) moiety which comprises a covalent bond between a silicon and a fluorine atom and which is optionally labelled with ¹⁸F; and
(d) one or more ligands which are capable of binding to prostate-specific membrane antigen (PSMA);
or a pharmaceutically or diagnostically acceptable salt or solvate thereof.

Ligand-SIFA conjugates comprising, within a single molecule four separate moieties:
(a) one or more ligands which are capable of binding to Fibroblast Activation Protein (FAP);
(b) a silicon-fluoride acceptor (SIFA) moiety which comprises a covalent bond between a silicon and a fluorine atom and which is optionally labelled with ¹⁸F;
(c) one or more chelating moieties (CM), optionally containing a chelated nonradioactive or radioactive cation; and
(d) one or more ligands which are capable of binding to prostate-specific membrane antigen (PSMA);
or a pharmaceutically or diagnostically acceptable salt or solvate thereof.

Suitably, the conjugates of the invention which comprise, within a single molecule, three or more separate moieties selected from FAP, SIFA and PSMA; FAP, SIFA and CM; and FAP, SIFA, PSMA and CM, as identified herein, may each independently be prepared from a conjugate of the invention which comprises, within a single molecule, two separate moieties selected from FAP and SIFA, as identified herein. Accordingly, the conjugates of the invention comprising, within a single molecule, two separate moieties selected form FAP and SIFA, as identified herein, may be regarded as intermediates for the synthesis of conjugates of the invention comprising, within a single molecule, three or more separate moieties selected from FAP, SIFA and PSMA; FAP, SIFA and CM; and FAP, SIFA, PSMA and CM, as identified herein.

Suitably, when the conjugates of the invention include more than one FAP ligand(s), each FAP ligand may be the same or different.

Suitably, said FAP ligand(s), in the conjugates of the invention, each independently comprise one or more four- to twelve-membered heterocyclic group(s), which heterocyclic group(s) contains at least one nitrogen atom and, optionally, one or more further heteroatoms selected from nitrogen, oxygen or sulphur. Suitably, each of said four- to twelve-membered heterocyclic group as identified herein represents a ring system which may be wholly, partly or non-aromatic in character. The term “four- to twelve-membered heterocyclic group(s)”, which each FAP ligand(s) may comprise, thus includes groups such as optionally substituted azetidinyl, pyrrolidinyl, pyrrolinyl, pyrrolyl, pyrazolyl, pyrazolinyl pyrazolidinyl, imidazolyl, imidazolinyl, imidazolidinyl, triazolyl, tetrazolyl, indolyl, oxadiazolyl, thiadiazolyl, oxatriazolyl, thiatriazolyl, pyridazinyl, pyrazinyl, morpholinyl, pyrimidinyl, purinyl, pyridinyl, piperidinyl, piperazinyl, quinolinyl, dihydroquinolinyl, tetrahydroquinolinyl, decahydroquinolinyl, isoquinolinyl, dihydroisoquinolinyl, decahydroisoquinolinyl, quinolzinyl, quinoxalinyl, phthalazinyl, quinazolinyl, cinnolinyl, naphthyridinyl, pyridopyrimidinyl, pyridopyrazinyl, and pteridinyl.

Suitably, said four- to twelve-membered heterocyclic group(s) identified herein, which each FAP ligand(s) may comprise, may be optional substituted with one or more optional substituent(s). Preferred optional one or more substituent(s) comprise halo, cyano, OH, B(OH)₂, CO₂H, C₁₋₆ alkyl, —O—C₁₋₆ alkyl, S—C₁₋₆ alkyl and optionally substituted amino. Highly preferred one or more optional substituent(s) are selected from halo, especially fluoro, and cyano.

Suitably, each FAP ligand(s), in the conjugates of the invention, independently comprise one or more four- to twelve membered heterocyclic group(s), as identified herein, which heterocyclic group(s) contain only one or more nitrogen atom(s) as the heteroatom(s).

Suitably, each FAP ligand(s), in the conjugates of the invention, independently comprise one or more four- to twelve-membered heterocyclic group(s), as identified herein, which group(s) contains at least one nitrogen atom and, optionally, one or more further nitrogen atoms.

Suitably, each FAP ligand(s), in the conjugates of the invention, independently comprise one or more five- to ten-membered heterocyclic group(s), which group(s) contains at least one nitrogen atom and, optionally, one or more further nitrogen atoms.

Suitably, each FAP ligand(s), in the conjugates of the invention, independently comprise one or more five- or ten-membered heterocyclic group(s), as identified herein, which group(s) contains at least one nitrogen atom and, optionally, one or more further nitrogen atoms.

Preferably, said one or more five- to ten-membered heterocyclic group(s), which group(s) contains at least one nitrogen atom and, optionally, one or more further nitrogen atoms, include only nitrogen as the heteroatom(s).

Suitably, each FAP ligand(s), in the conjugates of the invention, independently comprise one or more heterocyclic group(s) selected from optionally substituted pyrrolidinyl, pyrrolinyl, pyrrolyl, pyrazolyl, pyrazolinyl, pyrazolidinyl, imidazolyl, imidazolinyl, imidazolidinyl, triazolyl, tetrazolyl, indolyl, pyridazinyl, pyrazinyl, pyrimidinyl, purinyl, pyridinyl, piperidinyl, piperazinyl, quinolinyl, dihydroquinolinyl, tetrahydroquinolinyl, decahydroquinolinyl, isoquinolinyl, dihydroisoquinolinyl, decahydroisoquinolinyl, quinolzinyl, quinoxalinyl, phthalazinyl, quinazolinyl, cinnolinyl, naphthyridinyl, pyridopyrimidinyl, pyridopyrazinyl, and pteridinyl.

Suitably, each FAP ligand(s), in the conjugates of the invention, independently comprise one or more heterocyclic group(s) selected from optionally substituted pyrrolidinyl, pyrrolinyl, pyrrolyl, pyrazolyl, pyrazolinyl, pyrazolidinyl, imidazolyl, imidazolinyl, imidazolidinyl, triazolyl, tetrazolyl, quinolinyl, dihydroquinolinyl, tetrahydroquinolinyl, decahydroquinolinyl, isoquinolinyl, dihydroisoquinolinyl, decahydroisoquinolinyl, quinolzinyl, quinoxalinyl, phthalazinyl, quinazolinyl, cinnolinyl, naphthyridinyl, pyridopyrimidinyl, pyridopyrazinyl, and pteridinyl.

Suitably, each FAP ligand(s), in the conjugates of the invention, independently comprise one or more heterocyclic group(s) selected from optionally substituted pyrrolidinyl, pyrrolinyl, pyrrolyl, quinolinyl, dihydroquinolinyl, tetrahydroquinolinyl, decahydroquinolinyl, isoquinolinyl, dihydroisoquinolinyl, decahydroisoquinolinyl, quinoxalinyl, phthalazinyl, quinazolinyl, cinnolinyl, and naphthyridinyl.

Preferably, each FAP ligand(s), in the conjugates of the invention, independently comprise one or more heterocyclic group(s), selected from optionally substituted pyrrolidinyl, quinolinyl, isoquinolinyl, quinoxalinyl, phthalazinyl, quinazolinyl, cinnolinyl and naphthyridinyl, especially optionally substituted pyrrolidinyl, quinolinyl, isoquinolinyl, quinazolinyl

The silicon-fluoride acceptor (SIFA) moiety in the conjugates of the invention typically comprises an essentially C₄ to C₂₀ hydrocarbyl group which hydrocarbyl group is substituted by one or more silicon fluoride functional group(s), which silicon fluoride functional group(s) includes a silicon atom covalently bound to one or more fluorine atoms. Exemplary SIFA moieties are identified herein.

Suitably, the essentially C₄ to C₂₀ hydrocarbyl group of said SIFA moiety as identified herein comprises a C₆ to C₁₀ essentially hydrocarbyl group, preferably a C₆ to C₁₀ essentially hydrocarbyl group comprising an aryl group, more preferably a C₆ to C₁₀ essentially hydrocarbyl group comprising a phenyl ring.

Suitably, each of the one or more silicon fluoride functional group substituent(s) of the essentially C₄ to C₂₀, especially C₆ to C₁₀, hydrocarbyl moiety of the SIFA moiety as defined herein comprises a silicon atom covalently bound to one or more fluorine atoms and covalently bound to one or more C₃ to C₁₀ essentially hydrocarbyl groups. Suitably, the one or more silicon fluoride functional group substituent(s) comprises a silicon atom covalently bound to a single fluorine atom and covalently bound to two C₃ to C₁₀ essentially hydrocarbyl groups. Suitably, the one or more silicon fluoride functional group substituent(s) comprises a silicon atom covalently bound to a single fluorine atom and covalently bound to two C₃ to C₁₀ alkyl groups, which C₃ to C₁₀ alkyl groups may be the same or different.

A preferred SIFA moiety in the conjugates of the invention comprises a phenyl group which is substituted by one or more silicon fluoride functional group(s) identified herein, especially when said phenyl group is para-substituted by a silicon fluoride functional group identified herein. A highly preferred SIFA moiety in the conjugates of the invention comprises a phenyl group which is para-substituted by a silicon fluoride functional group comprising a silicon atom covalently bound to a single fluorine atom and covalently bound to two C₃ to C₁₀ alkyl groups, which C₃ to C₁₀ alkyl groups may be the same or different.

In some embodiments, the conjugates of the invention may include (c) one or more optional chelating moieties (CM). Suitably, when the conjugates of the invention include more than one chelating moiety (CM), each CM may be the same or different.

Exemplary chelating moieties which may be present in the conjugates of the invention are identified herein. Preferably, when the conjugates of the invention include one or more of said optional chelating moieties (CM), each of said one or more chelating moieties is independently selected from TRAP, DOTA and DOTAGA, especially DOTA and DOTAGA.

Suitably, the one or more optional chelating moieties (CM) may each independently include a radioactive cation or a non-radioactive cation, preferably a radioactive metal or non-radioactive metal cation as identified herein. Preferably, when the one or more chelating moieties includes a radioactive or non-radioactive cation, the cation is selected from a Ga, Cu, Lu, Y and Ac cation, especially ⁶⁸Ga, ⁶⁴Cu, ¹⁷⁷Lu, ⁹⁰Y and ²²⁵Ac cation. In some preferred embodiments, the radioactive or non-radioactive cation is a cation of indium, technetium, gallium, gadolinium, copper, lutetium, or rhenium. In some preferred embodiments, the radioactive or non-radioactive cation is ¹¹¹In, ^{99m}Tc, ⁶⁴Cu, ⁶⁷Cu, ⁶⁷Ga or ⁶⁸Ga. In some embodiments, the one or more optional chelating moieties (CM) may include a cation that bonds with a radioactive element. In some embodiments, the one or more optional chelating moieties (CM) may include a cation that bonds with ¹⁸F. In some embodiments, the one or more optional chelating moieties (CM) may include Al¹⁸F₃ or Sc¹⁸F₃. Suitably, the one or more chelating moieties (CM) may be labelled with radioactive cation, such as ⁶⁸Ga or ⁶⁴Cu, for use in diagnostics as a radiotracer. Alternatively, the one or more chelating moieties may be labelled with a therapeutic isotope, such as ¹⁷⁷Lu, ⁹⁰Y or ²²⁵Ac.

For some embodiments of the present invention, the conjugate comprises ¹⁸F, and for some embodiments of the present invention, the conjugate comprises one or more other radioactive isotopes (e.g. associated with the chelating moiety). In some embodiments of the invention, the conjugate comprises both ¹⁸F and one or more other radioactive isotopes.

Accordingly, conjugates of the invention which include a SIFA moiety radiolabeled with ¹⁸F and one or more chelating moieties (CM) which include an appropriate radioactive cation allows the conjugates of the invention to be employed as “paired” tracers to bridge diagnostic and therapeutic radiopharmaceutical applications.

In some embodiments, the conjugates of the invention include (d) one or more optional PSMA ligands. Suitably, when the conjugates of the invention include more than one PSMA ligand(s), each PSMA ligand may be the same or different.

Exemplary PSMA ligands which may optionally be present in the conjugates of the invention are identified herein and/or disclosed in WO2019/020831, WO2020/157177 and WO2020/157184.

Suitably, the one or more optional PSMA ligand(s) (d), when present, is each independently selected from a structure represented by formulae PSMA 1, PSMA 2, PSMA 3, PSMA 4 and PSMA 5 as identified herein.

Suitably, the conjugates of the invention may be represented by a conjugate of formula I

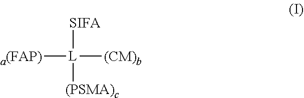

or a pharmaceutically or diagnostically acceptable salt or solvate thereof, wherein:
(i) each FAP independently represents a ligand which is capable of binding to Fibroblast Activation Protein (FAP) as identified herein;
(ii) L represents an optionally substituted linker group as identified herein;
(iii) SIFA represents a silicon-fluoride acceptor (SIFA) moiety which comprises a covalent bond between a silicon and a fluorine atom, and which SIFA moiety is optionally labelled with ¹⁸F, as identified herein;
(iv) each CM independently represents a chelating moiety, optionally containing a chelated nonradioactive cation or radioactive cation, as identified herein;
(v) each PSMA independently represents a ligand which is capable of binding to prostate-specific membrane antigen (PSMA) as identified herein;
a is an integer from 1 to 3; b is an integer from 0 to 2; and, c is an integer from 0 to 2.

Suitably, each FAP ligand in a compound of formula (I) may be the same or different, preferably each FAP ligand is identical.

Suitably, when present, each CM in a compound of formula (I) may be the same or different, preferably each CM is identical.

Suitably, when present, each PSMA ligand in a compound of formula (I) may be the same or different, preferably each PSMA ligand is identical.

Suitably, L, the optionally substituted linker group in the conjugate of formula (I), and formulae (IA), (IB), (IC), (ID), (IE), (IF), (IG) identified hereinafter, represents a multivalent organic linker group which is capable of forming a separate covalent bond with (a) each of said one or more FAP ligands, with (b) said silicon-fluoride acceptor (SIFA), with (c) each of said one or more optional chelating moieties (CM), when present, and with (d) each of said one or more optional PSMA ligands (d), when present. Suitably, each of said one or more FAP ligand(s), said SIFA, said one or more optional CM(s), when present, and said one or more optional PSMA ligand(s), when present, are each independently covalently bonded to L.

Suitably, each of said one or more FAP ligand(s), said SIFA, said one or more optional CM(s), when present, and said one or more optional PSMA ligand(s), when present, may each independently be covalently bonded to a common atom (i.e. at the same position) of the linker group L. Suitably, each of said one or more FAP ligand(s), said SIFA, said one or more optional CM(s), when present, and said one or more optional PSMA ligand(s), when present, may each independently be covalently bonded at one or more different atoms (i.e. one or more different positions) of the linker group L. Preferably, each of said one or more FAP ligand(s), said SIFA, said one or more optional CM(s), when present, and said one or more optional PSMA ligand(s), when present, are each independently covalently bonded at one or more different positions of the linker group L.

Suitably, L in the conjugate of formula (I), and formulae (IA), (IB), (IC), (ID), (IE), (IF), (IG) identified hereinafter, represents an optionally substituted multivalent linking group comprising a structure selected from an oligoamide, an oligoether, an oligothioether, an oligoester, an oligothioester, an oligourea, an oligo(ether-amide), an oligo(thioether-amide, an oligo(thioester-amide), an oligo(urea-amide), an oligo(thioether-ester), an oligo(thioether-thioester), an oligo(thioetherurea), an oligo(ester-thioester), an oligo(ester-urea), an oligo(thioester-urea).

Preferably, L in the conjugate of formula (I), and formulae (IA), (IB), (IC), (ID), (IE), (IF), (IG) identified hereinafter, represents an optionally substituted multivalent linking group having a structure selected from an oligoamide and oligo(ester-amide).

Suitably, the optional substituents of the multivalent linking group may be selected from —OH, —OCH₃, —COOH, —COOCH₃, —NH₂, and —NHC(NH)NH₂

The term “oligo” as used in oligoamide, oligoether, oligothioether, oligoester, oligothioester, oligourea, oligo(ether-amide), oligo(thioether-amide), oligo(ester-amide), oligo(thioester-amide), oligo(urea-amide), oligo(ether-thioether), oligo(ether-ester), oligo(ether-thioester), oligo (ether-urea), oligo(thioether-ester), oligo(thioether-thioester), oligo(thioether-urea), oligo(ester-thioester), oligo(ester-urea), and oligo(thioester-urea) is preferably to be understood as referring to a group wherein 2 to 20, more preferably wherein 2 to 10 subunits are linked by the type of bonds specified in the same terms. As will be understood by the skilled reader, where two different types of bonds are indicated in brackets, both types of bonds are contained in the concerned group (e.g. in “oligo (ester-amide)”, ester bonds and amide bonds are contained).

It is preferred that the linker group has a structure selected from an optionally substituted oligoamide and oligo(ester-amide) and comprises a total of 1 to 5, more preferably a total of 1 to 3, and most preferably a total of 1 or 2 amide and/or ester bonds, preferably amide bonds, within its backbone.

The term oligoamide therefore includes a moiety having a chain of CH₂ or CHR groups interrupted with groups selected from NHCO or CONH. Each occurrence of the R moiety is an optional substituent selected from, for example, —OH, —OCH₃, —COOH, —COOCH₃, —NH₂, and —NHC(NH)NH₂.

Suitably, said (a) one or more FAP ligands, said (b) SIFA, said (c) one or more optional CM, when present, and said (d) one or more optional PSMA, when present, may each independently be covalently bonded to the linker group (L) by a covalent bond which covalent bond forms a part of a functional group, for example an ether group, ester group, thioester group, thioether group, amide group, carbamate group.

Preferred conjugates of formula I include conjugates wherein:
(i) each FAP ligand(s) independently comprise one or more four- to twelve-membered heterocyclic group(s), as identified herein, which heterocyclic group(s) contains at least one nitrogen atom and, optionally, one or more further heteroatoms selected from nitrogen, oxygen or sulphur;
(ii) L represents an optionally substituted linker group as identified herein;
(iii) SIFA comprises an essentially C₄ to C₂₀ hydrocarbyl group which hydrocarbyl group is substituted by one or more silicon fluoride functional group(s), as identified herein, which silicon fluoride group(s) includes a silicon atom covalently bound to one or more fluorine atoms, and which SIFA moiety is optionally labelled with ¹⁸F; and,
(iv) CM, (v) PSMA, a, b and c are each as defined for a compound of formula I.

More preferred conjugates of formula I include conjugates wherein:
(i) each FAP ligand(s) independently comprise one or more five- or ten-membered heterocyclic group(s), as identified herein, which heterocyclic group(s) contains at least one nitrogen atom and, optionally, one or more further nitrogen atoms, and wherein each of said one or more heterocyclic group(s) include only nitrogen heteroatom(s);
(ii) L represents an optionally substituted linker group as identified herein;
(iii) SIFA comprises a C₆ to C₁₀ essentially hydrocarbyl group comprising an aryl group, which aryl group is substituted by one or more silicon fluoride functional group(s) as identified herein, and which silicon fluoride group(s) includes a silicon atom covalently bound to one or more fluorine atoms, and which SIFA moiety is optionally labelled with ¹⁸F; and,
(iv) CM, (v) PSMA, a, b and c are each as defined for a conjugate of formula I.

Even more preferred conjugates of formula I include conjugates wherein:
(i) each FAP ligand(s) independently comprise one or more heterocyclic group(s) selected from optionally substituted pyrrolidinyl, pyrrolinyl, pyrrolyl, quinolinyl, dihydroquinolinyl, tetrahydroquinolinyl, decahydroquinolinyl, isoquinolinyl, dihydroisoquinolinyl, decahydroisoquinolinyl, quinoxalinyl, phthalazinyl, quinazolinyl, cinnolinyl, and naphthyridinyl;
(ii) L represents an optionally substituted linker group as identified herein;
(iii) SIFA comprises a C₆ to C₁₀ essentially hydrocarbyl group comprising a phenyl group, which phenyl group is substituted, preferably para-substituted, by one or more silicon fluoride functional group(s) as identified herein, and which silicon fluoride group(s) includes a silicon atom covalently bound to one or more fluorine atoms and covalently bound to one or more C₃ to C₁₀ essentially hydrocarbyl groups, and which SIFA moiety is optionally labelled with ¹⁸F; and,
(iv) CM, (v) PSMA, a, b and c are each as defined for a conjugate of formula I.

Even more preferred conjugates of formula I include conjugates wherein:
(i) each FAP ligand(s) independently comprise one or more heterocyclic group(s) selected from optionally substituted pyrrolidinyl, quinolinyl, isoquinolinyl, quinoxalinyl, phthalazinyl, quinazolinyl, cinnolinyl and naphthyridinyl;
(ii) L represents an optionally substituted linker group as identified herein;
(iii) SIFA comprises a C₆ to C₁₀ essentially hydrocarbyl group comprising a phenyl group, which phenyl group is para-substituted by a silicon fluoride functional group comprising a silicon atom covalently bound to a single fluorine atom and covalently bound to two C₃ to C₁₀ alkyl groups, and which SIFA moiety is optionally labelled with ¹⁸F; and,
(iv) CM, (v) PSMA, a, b and c are each as defined for a conjugate of formula I.

Even more preferred conjugates of formula I include conjugates wherein:
(i) each FAP ligand(s) independently comprise one or more heterocyclic group(s) selected from optionally substituted pyrrolidinyl, quinolinyl, isoquinolinyl, and quinazolinyl;
(ii) L represents an optionally substituted linker group as identified herein;
(iii) SIFA comprises a C₆ to C₁₀ essentially hydrocarbyl group comprising a phenyl group, which phenyl group is para-substituted by a silicon fluoride functional group comprising a silicon atom covalently bound to a single fluorine atom and covalently bound to two C₃ to C₁₀ alkyl group, and which SIFA moiety is optionally labelled with ¹⁸F; and,
(iv) CM, (v) PSMA, a, b and c are each as defined for a conjugate of formula I.

It will be appreciated, that in the conjugates of formula I, and similarly the conjugates of formulae IA to IG identified herein, that each of said one or more FAP ligand(s), said SIFA, said one or more optional CM(s), when present, and said one or more optional PSMA ligand(s), when present, may each independently be covalently bonded to a common atom (i.e. the same position) of the linker group L or each independently covalently bonded at one or more different atoms (i.e. one or more different positions) of the linker group L.

In a preferred embodiment, conjugates of the invention include conjugates of formula I where a is 1 or 2, b is 0 and c is 0 and include conjugates of formulae IA and IB:

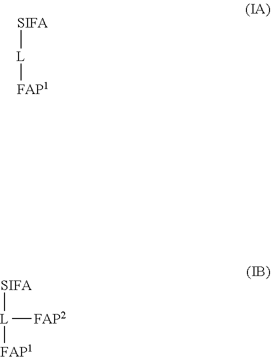

or a pharmaceutically or diagnostically acceptable salt or solvate thereof, wherein:
FAP¹ and FAP², when present, each independently represent a FAP ligand as defined for a conjugate of formula I; and, SIFA and L are each as defined for a conjugate of formula I.

Preferred conjugates of formulae IA and IB include conjugates wherein:
(i) FAP¹ and FAP², when present, each independently represents a FAP ligand as defined for a preferred conjugate of formula I, as identified herein;
(iii) L represents an optionally substituted linker group as identified herein; and,
(iii) SIFA is as defined for a preferred conjugate of formula I, as identified herein.

Suitably, highly preferred conjugates of formulae IA and IB include conjugates wherein.
(i) FAP¹ and FAP², when present, each independently represent a FAP ligand comprising one or more heterocyclic group(s) selected from optionally substituted pyrrolidinyl, quinolinyl, isoquinolinyl, and quinazolinyl;
(ii) L represents an optionally substituted linker group as defined for a conjugate of formula I;
(iii) SIFA comprises a C₆ to C₁₀ essentially hydrocarbyl group comprising a phenyl group, which phenyl group is para-substituted by a silicon fluoride functional group comprising a silicon atom covalently bound to a single fluorine atom and covalently bound to two C₃ to C₁₀alkyl groups, and which SIFA moiety is optionally labelled with ¹⁸F.

In an alternative preferred embodiment, conjugates of the invention include conjugates of formula I where a is 1 or 2, b is 1 and c is 0 and include conjugates of formulae IC and ID:

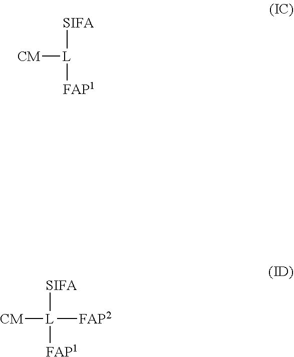

or a pharmaceutically or diagnostically acceptable salt or solvate thereof, wherein:
FAP¹ and FAP², when present, SIFA and L are as defined for a conjugate of formulae IA and IB, and CM is as defined for a conjugate of formula (I).

Preferred conjugates of formulae IC and ID include conjugates wherein CM is selected from TRAP, DOTA and DOTAGA, which CM is optionally substituted with a radioactive metal cation or non-radioactive cation identified herein, especially optionally substituted with a ⁶⁸Ga, ⁶⁴Cu, ¹⁷⁷Lu, ⁹⁰Y or ²²⁵Ac cation.

More preferred conjugates of formulae IC and ID include conjugates wherein:
(i) FAP¹ and FAP², when present, each independently represents a FAP ligand as defined for a preferred conjugate of formula IA and IB, as identified herein;
(ii) SIFA is as defined for a preferred conjugate of formula IA, as identified herein;
(iii) L represents an optionally substituted linker group as defined for a conjugate of formula I; and,
(iv) CM is selected from TRAP, DOTA and DOTAGA, which CM is optionally substituted with a radioactive metal cation or non-radioactive cation identified herein, especially optionally substituted with a ⁶⁸Ga, ⁶⁴Cu, ¹⁷⁷Lu, ⁹⁰Y or ²²⁵Ac cation.

Suitably, highly preferred conjugates of formulae IC and ID include conjugates wherein:
(i) FAP¹ and FAP², when present, each independently represent a FAP ligand comprising one or more heterocyclic group(s) selected from optionally substituted pyrrolidinyl, quinolinyl, isoquinolinyl, and quinazolinyl;
(ii) L represents an optionally substituted linker group as identified herein;
(iii) SIFA comprises a C₆ to C₁₀ essentially hydrocarbyl group comprising a phenyl group, which phenyl group is para-substituted by a silicon fluoride functional group comprising a silicon atom covalently bound to a single fluorine atom and covalently bound to two C₃ to C₁₀ alkyl groups, and which SIFA moiety is optionally labelled with ¹⁸F; and,
(iv) CM is selected from TRAP, DOTA and DOTAGA, which CM is optionally substituted with a radioactive metal cation or non-radioactive cation identified herein, especially optionally substituted with a ⁶⁸Ga, ⁶⁴Cu, ¹⁷⁷Lu, ⁹⁰Y or ²²⁵Ac cation.

In a further alternative preferred embodiment, conjugates of the invention include conjugates of formula I where a is 1, b is 1 and c is 1 and include conjugates of formula IE:

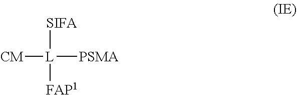

or a pharmaceutically or diagnostically acceptable salt or solvate thereof, wherein:
FAP¹, SIFA, CM and L are each as defined for a conjugate of formula IC, and PSMA is as defined for a compound of formula I.

Preferred conjugates of formula IE include conjugates wherein PSMA is selected from a structure represented by formulae PSMA 1, PSMA 2, PSMA 3, PSMA 4 and PSMA 5 as identified herein.

More preferred conjugates of formula IE include conjugates wherein PSMA is selected from a structure represented by formulae PSMA 1, PSMA 2, PSMA 3, PSMA 4 and PSMA 5 as identified herein and CM is selected from TRAP, DOTA and DOTAGA, which CM is optionally substituted with a radioactive metal cation or non-radioactive cation identified herein, especially a ⁶⁸Ga, ⁶⁴Cu, ¹⁷⁷Lu, ⁹⁰Y or ²²⁵Ac cation.

Even more preferred conjugates of formula IE include conjugates wherein:
(i) FAP¹ independently represents a FAP ligand as defined for a preferred conjugate of formula IC, as identified herein;
(iii) L represents an optionally substituted linker group, as identified herein;
(iii) SIFA is as defined for a preferred conjugate of formula IC, as identified herein;
(iv) PSMA is selected from a structure represented by formulae PSMA 1, PSMA 2, PSMA 3, PSMA 4 and PSMA 5 as identified herein; and
(v) CM is selected from TRAP, DOTA and DOTAGA, which CM is optionally substituted with a radioactive metal cation or non-radioactive cation identified herein, especially a ⁶⁸Ga, ⁶⁴Cu, ¹⁷⁷Lu, ⁹⁰Y or ²²⁵Ac cation.

Suitably, highly preferred conjugates of formula IE include conjugates wherein:
(i) FAP¹ independently represents a FAP ligand comprising one or more heterocyclic group(s) selected from optionally substituted pyrrolidinyl, quinolinyl, isoquinolinyl, and quinazolinyl;
(ii) L represents an optionally substituted linker group as identified herein;
(iii) SIFA comprises a C₆ to C₁₀ essentially hydrocarbyl group comprising a phenyl group, which phenyl group is para-substituted by a silicon fluoride functional group comprising a silicon atom covalently bound to a single fluorine atom and covalently bound to two C₃ to C₁₀ alkyl groups, and which SIFA moiety is optionally labelled with ¹⁸F;
(iv) PSMA is selected from a structure represented by formulae PSMA 1, PSMA 2, PSMA 3, PSMA 4 and PSMA 5 as identified herein; and
(v) CM is selected from TRAP, DOTA and DOTAGA, which CM is optionally substituted with a radioactive metal cation or non-radioactive cation identified herein, especially optionally substituted with a ⁶⁸Ga, ⁶⁴Cu, ¹⁷⁷Lu, ⁹⁰Y or ²²⁵Ac cation.

In a further alternative embodiment, alternative preferred conjugates of the invention include conjugates of formula I where a is 1 or 2, b is 0 and c is 1 and include conjugates of formulae IF and IG:

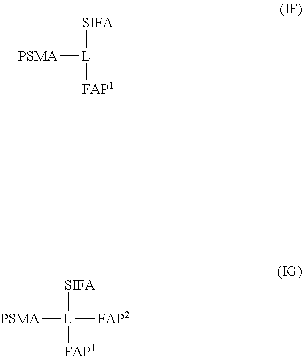

or a pharmaceutically or diagnostically acceptable salt or solvate thereof, wherein:
FAP¹ and FAP², when present, SIFA and L are as defined for a conjugate of formulae IA and IB, and PSMA is as defined for a conjugate of formula IE.

Preferred conjugates of formulae IF and IG include conjugates wherein:
(i) FAP¹ and FAP², when present, each independently represents a FAP ligand as defined for a preferred conjugate of formulae IA and IB, as identified herein;
(iii) L represents an optionally substituted linker group as identified herein;
(iii) SIFA is as defined for a preferred conjugate of formulae IA and IB, as identified herein; and,
(iv) PSMA is selected from a structure represented by formulae PSMA 1, PSMA 2, PSMA 3, PSMA 4 and PSMA 5 as identified herein.

Suitably, highly preferred conjugates of formulae IF and IG include conjugates wherein:
(i) FAP¹ and FAP², when present, each independently represent a FAP ligand comprising one or more heterocyclic group(s) selected from optionally substituted pyrrolidinyl, quinolinyl, isoquinolinyl, and quinazolinyl;
(ii) L represents an optionally substituted linker group as identified herein;
(iii) SIFA comprises a C₆ to C₁₀ essentially hydrocarbyl group comprising a phenyl group, which phenyl group is para-substituted by a silicon fluoride functional group comprising a silicon atom covalently bound to a single fluorine atom and covalently bound to two C₃ to C₁₀ alkyl groups, and which SIFA moiety is optionally labelled with ¹⁸F; and,
(iv) PSMA is selected from a structure represented by formulae PSMA 1, PSMA 2, PSMA 3, PSMA 4 and PSMA 5 as identified herein.

Highly preferred conjugates of the invention include conjugates of formula I where a is 1 or 2, b is 0 or 1, and c is 0 as represented by conjugates of formulae IA, IB, IC and ID. Especially preferred conjugates of the invention include conjugates of formula I where a is 1, b is 1 and c is 0 as represented by conjugates of formula IC.

Conjugates of the invention may comprise a single FAP ligand as exemplified by conjugates of formulae IA, IC, IE and IF, two FAP ligands as exemplified by conjugates of formulae IB, ID and IG, or three or more FAP ligands. When a conjugate includes two or more FAP ligands, each of said FAP ligands may be the same or different, preferably each of said FAP ligands is identical. Thus, FAP¹ is preferably the same as FAP² in conjugates of formulae IB, IC and IG.

In some embodiments of the present invention, conjugates may comprise one or more ligands which are capable of binding to PSMA in addition to one or more of ligands which are capable of binding to FAP, as exemplified by conjugates of formulae IE, IF and IG. Such conjugates are useful as “dual” radio-therapeutics and/or radio-diagnostics for medical indications associated with elevated FAP expression and elevated prostate-specific membrane antigen (PSMA) expression.

According to a further aspect of the invention there is provided processes for the preparation of the conjugates of the invention, as illustrated below.

The following processes are illustrative of the general synthetic procedure which may be adopted in order to obtain the conjugates of the invention.

Conjugates of the invention comprising, within a single molecule, two separate moieties: (a) one or more ligand(s) which is capable of binding to Fibroblast Activation Protein (FAP); and, (b) a silicon-fluoride acceptor (SIFA) moiety which comprises a covalent bond between a silicon and a fluorine atom, as exemplified by conjugates of formulae IA and IB, may be prepared by coupling each of (a) said one or more FAP ligands and (b) said SIFA moiety to a common linker group L to form a single molecule.

Conjugates of the invention comprising, within a single molecule, three separate moieties: (a) one or more ligands which are capable of binding to Fibroblast Activation Protein (FAP); (b) a silicon-fluoride acceptor (SIFA) moiety which comprises a covalent bond between a silicon and a fluorine atom; and, (c) one or more chelating moieties (CM), optionally containing a chelated nonradioactive cation or radioactive cation, as exemplified by conjugates of formulae IC and ID may be prepared by coupling each of (a) said one or more FAP ligands, (b) said SIFA moiety and (c) said one or more chelating moieties (CM), to a common linker group L to form a single molecule.

Conjugates of the invention comprising, within a single molecule, three separate moieties: (a) one or more ligands which are capable of binding to Fibroblast Activation Protein (FAP); (b) a silicon-fluoride acceptor (SIFA) moiety which comprises a covalent bond between a silicon and a fluorine atom; and, (d) one or more ligands which are capable of binding to prostate-specific membrane antigen (PSMA), as exemplified by conjugates of formulae IF and IG, may be prepared by coupling each of (a) said one or more FAP ligands, (b) said SIFA moiety and (d) said one or more PSMA ligands to a common linker group L to form a single molecule.

Conjugates of the invention comprising, within a single molecule, four separate moieties: (a) one or more ligands which are capable of binding to Fibroblast Activation Protein (FAP); (b) a silicon-fluoride acceptor (SIFA) moiety which comprises a covalent bond between a silicon and a fluorine atom; (c) one or more chelating moieties (CM), optionally containing a chelated nonradioactive or radioactive cation, and, (d) one or more ligands which are capable of binding to prostate-specific membrane antigen (PSMA), as exemplified by conjugates of formula IE, may be prepared by coupling each of (a) said one or more FAP ligands, (b) said SIFA moiety to a common linker group, (c) said one or more chelating moieties (CM) and (d) said one or more PSMA ligands to a common linker group L to form a single molecule.

A preferred process for preparing the conjugates of the invention comprises providing a conjugate precursor compound comprising a FAP ligand, as identified herein, covalently bound to the common linker group L and subsequently coupling (b) said SIFA moiety, (c) said one or more optional chelating moieties (CM), said one or more optional PSMA ligand(s), and said one or more further optional additional FAP ligand(s) to the linker group conjugate precursor compound.

The coupling reactions of said one or more FAP ligand(s) and said SIFA moiety, and, when present, said one or more optional CM moieties and/or said one or more optional PSMA ligand(s), to the linker group may be achieved by conventional bond forming techniques which are well known to those skilled in the art, for example the use of conventional amide, ester, ether, thioether, thioester bond forming techniques. Typical procedures that may be employed include those described herein. Further, it will be appreciated, that the coupling reactions of said one or more FAP ligands and said SIFA moiety, and, when present, said optional one or more CM moieties and/or said optional one or more PSMA ligand(s), to the linker group may be performed in any order. The conjugates of the invention may be isolated from their reaction mixtures using conventional techniques, for example crystallization, chromatography including column chromatography and HPLC.

The conjugates of the invention are useful in a variety of therapeutic and/or diagnostic areas associated with elevated FAP expression, including the treatment and/or diagnosis of various cancers in an animal or human subject. Suitably, a therapeutically and/or diagnostically effective amount of the conjugate(s) of the invention is administered to the animal or human subject.

The conjugates of the invention may be useful in the treatment or diagnosis of medical indications associated with elevated FAP expression in human tissue. The conjugates of the invention may be useful in the treatment or diagnosis of cancer.

The conjugates of the invention may be useful in the diagnosis or treatment of a disease characterized by overexpression of fibroblast activation protein (FAP) in an animal or a human subject. The disease characterized by overexpression of fibroblast activation protein (FAP) may be selected from the group consisting of cancer, chronic inflammation, atherosclerosis, fibrosis, tissue remodelling and keloid disorder. The cancer may be selected from the group consisting of breast cancer, pancreatic cancer, small intestine cancer, colon cancer, rectal cancer, lung cancer, head and neck cancer, ovarian cancer, hepatocellular carcinoma, esophageal cancer, hypopharynx cancer, nasopharynx cancer, larynx cancer, myeloma cells, bladder cancer, cholangiocellular carcinoma, clear cell renal carcinoma, neuroendocrine tumor, oncogenic osteomalacia, sarcoma, CUP (carcinoma of unknown primary), thymus carcinoma, desmoid tumors, glioma, astrocytoma, cervix carcinoma and prostate cancer. Thus, the conjugates of the invention having a FAP binding moiety (i.e. FAP ligand) can be used in the diagnosis, imaging or treatment of a cancer having FAP expression.

Suitably, when the conjugates of the invention further include said optional (d) one or more PSMA ligands, said conjugates of the invention may be useful in the diagnosis or treatment of a disease characterized by overexpression of fibroblast activation protein (FAP), or overexpression of PSMA, or overexpression of both FAP and PSMA, in an animal or a human subject. The disease characterized by overexpression of PSMA includes not only prostate cancer. Non-prostate cancers known to demonstrate PSMA expression include breast, lung, colorectal, and renal cell carcinoma. Thus any conjugate of the invention identified herein having a PSMA binding moiety (i.e. PSMA ligand) can be used in the diagnosis, imaging or treatment of a cancer having PSMA expression.

The conjugates of the invention may be useful in (i) the detection of smaller primary tumors, thus allowing earlier diagnosis, (ii) the detection of smaller metastasis, thus affording a better assessment of tumor stage, (iii) providing precise intra-operative guidance facilitating complete surgical removal of tumor tissue, (iv) providing better differentiation between inflammation and tumor tissue, (v) providing more precise staging of patients with tumors, (vi) providing better follow up of tumor lesions after antitumor therapy, and (vii) as theranostic agents for diagnosis and therapy. Furthermore, the conjugates of the invention can be used for the diagnosis and treatment of non-malignant diseases such as chronic inflammation, atherosclerosis, fibrosis, tissue remodelling and keloid disorders.

In a further aspect, the present invention provides a pharmaceutical composition comprising or consisting of one or more conjugates of the invention as disclosed herein. Suitably, the pharmaceutical composition may include pharmaceutically acceptable carriers, excipients and/or diluents.

In a further aspect, the present invention provides a diagnostic composition comprising or consisting of one or more conjugates of the invention as disclosed herein. Suitably, the diagnostic composition may include diagnostically acceptable carriers, excipients and/or diluents.

In a further aspect, the present invention provides said one or more conjugates of the invention, or a composition, especially pharmaceutical or diagnostic composition, comprising said conjugate(s) of the invention, for use in medicine.

Preferred uses in medicine are in nuclear medicine such as nuclear diagnostic imaging and/or staging, also named nuclear molecular imaging, and/or targeted radiotherapy of disease associated with an overexpression, preferably of FAP on the diseased tissue.

It will be appreciated that when the conjugates of the invention further include said optional (d) one or more PSMA ligands said preferred uses in medicine may further extend to nuclear diagnostic imaging and/or staging, and/or targeted radiotherapy of disease associated with an overexpression, preferably of FAP and/or PSMA on the diseased tissue. Accordingly, such conjugates of the invention are useful for combined diagnostic imaging and/or staging, and/or combined targeted radiotherapy of disease associated with an overexpression, preferably of both FAP and PSMA on the diseased tissue.

In a further aspect, the present invention provides said one or more conjugates of the invention as defined herein, or a composition comprising said conjugate(s) of the invention as defined herein, for use in the treatment of cancer in an animal or human subject.

In a further aspect, the present invention provides said one or more conjugates of the invention as defined herein, or a composition comprising said conjugate(s) of the invention as defined herein, for use in the treatment of a disease characterized by overexpression of FAP in an animal or human subject, especially for use in the treatment of cancer in an animal or human subject characterized by overexpression of FAP.

In a further aspect, the present invention provides said one or more conjugates of the invention as defined herein including said optional (d) one or more PSMA ligands, or a composition comprising said conjugate(s) of the invention, for use in the treatment of a disease characterized by overexpression of FAP, or overexpression of PSMA, or overexpression of both FAP and PSMA, in an animal or human subject, especially for use in the treatment of cancer in an animal or human subject characterized by overexpression of both FAP and PSMA.

In a further aspect, the present invention provides said one or more conjugates of the invention as defined herein, or a composition comprising said conjugate(s) of the invention, for use as a diagnostic or imaging agent in an animal or human subject, especially for use as a diagnostic or imaging agent of a disease, preferably cancer, associated with an overexpression of FAP.

In a further aspect, the present invention provides said one or more conjugates of the invention as defined herein including said optional (d) one or more PSMA ligands, or a composition comprising said conjugate(s) of the invention as defined herein, for use as a diagnostic or imaging agent in an animal or human subject, especially for use as a diagnostic or imaging agent of a disease, preferably cancer, associated with an overexpression of FAP and/or overexpression of PSMA

Suitably, in a further aspect the present invention provides said one or more conjugates of the invention as defined herein, or a composition comprising said conjugate(s) of the invention, for use as a cancer diagnostic or imaging agent.

Preferred indications are the detection or staging of cancer associated with an overexpression of FAP, Prostate cancer is a particularly preferred indication.

Suitably, in a further aspect the present invention provides a method for treatment of the human or animal body by surgery or therapy or a diagnostic method practiced on the human or animal body comprising administering a therapeutically or diagnostically effective amount of said one or more conjugates of the invention as defined herein, or a composition comprising said conjugate(s) of the invention, as defined herein, to a human or animal subject. Suitably, the method for treatment is of cancer.

### Definitions

The following definitions are provided for purpose of illustration and not limitation.

“FAP ligand” means a chemical moiety which includes one or more functional group(s), e.g. organic functional group(s), which is capable of binding to Fibroblast Activation Protein (FAP) expressed in mammalian, especially human, tissue. Exemplary FAP ligand(s) of the conjugates of the invention are identified herein.

“SIFA moiety” means a silicon-fluoride acceptor moiety which comprises a covalent bond between a silicon and a fluorine atom and which is optionally labelled with ¹⁸F. Exemplary SIFA moieties of the conjugates of the invention are identified herein.

“Chelating moiety” (CM) includes, amongst other things: (i) a macrocyclic ring structure with 8 to 20 ring atoms of which 2 or more are heteroatoms selected from oxygen atoms and nitrogen atoms; (ii) an acyclic, open chain chelating structure with 8 to 20 main chain atoms of which 2 or more are heteroatoms selected from oxygen atoms and nitrogen atoms; (iii) a branched chelating structure containing a quaternary carbon atom. Exemplary chelating moieties of the conjugates of the invention are identified herein.

“PSMA ligand” means a chemical moiety which includes one or more functional group(s), e.g. organic functional group(s), which is capable of binding to Prostrate Specific Membrane Antigen expressed in mammalian, especially human, tissue. Exemplary PSMA ligands in the conjugates of the invention are identified herein and/or disclosed in WO2019/020831, WO2020/157177 and WO2020/157184.

“Hydrocarbyl” means a group or radical that contains carbon and hydrogen atoms and that is bonded to the rest of the molecule via a carbon atom. It may contain hetero atoms, i.e. atoms other than carbon and hydrogen, provided they do not alter the essentially hydrocarbon nature and characteristics of the group. Preferred hydrocarbyl groups and radicals include only hydrogen and carbon. Suitably, the term hydrocarbyl embraces aliphatic and aromatic groups and radicals. Preferred hydrocarbyl groups comprise aliphatic groups and radicals, such as alkyl, alkylene, alkenyl groups and radicals.

“Alkyl” refers to a monovalent hydrocarbyl group containing no double or triple bonds. The alkyl group many be linear, branched, cyclic, acyclic, and/or part cyclic/acyclic. The alkyl group may be optional substituted with one or more substituents. Suitably, the term C₁ to C₁₀ alkyl group covers methyl, ethyl, propyl, isopropyl, n-butyl, i-butyl, t-butyl, pentyl, cyclopentyl, hexyl, cyclohexyl, heptyl, octyl, nonyl and decyl. Preferred alkyl groups represent acyclic alkyl groups.

“Aryl” refers to six to ten membered carbocyclic aromatic groups, such as phenyl and naphthyl. Each “aryl” group identified herein may be optionally substituted with one or more substituents selected from halo, cyano, nitro, C₁ to C₆ alkyl, C(O)R²¹, C(O)OR²², C(O)NR²³R²⁴, NR²⁵R²⁶, wherein R²¹, R²², R²³, R²⁴, R²⁵ and R²⁶ each independently represent hydrogen or C₁-C₆ alkyl.

“Halo” refers to fluoro, chloro, bromo and iodo.

“Comprising” or any cognate word specifies the presence of stated features, steps or integers or components, but does not preclude the presence or addition of one or more other features, steps, integers, components or groups thereof; the expressions “consists of” and “consists essentially of” or cognates may be embraced within “comprises” or cognates, wherein “consists essentially of” permits inclusion of substances not materially affecting the characteristics of the compound, composition or other feature to which it refers.

The conjugates of the invention may exhibit tautomerism. All tautomeric forms of the conjugates of the invention are included within the scope of the invention.

The conjugates of the invention may also contain one or more asymmetric carbon atoms and may exhibit optical and/or diastereoisomerim. Diastereoisomers may be separated by conventional technique, e.g. by fractional crystallisation or chromatography. The various stereoisomers may be isolated by separation of a racemic mixture or other mixture using conventional techniques e.g. fractional crystallisation and High performance liquid chromatography (HPLC). All stereoisomers are include within the scope of the conjugates of the invention.

Further, it is understood that any upper and lower quantity, range and ratio limits set forth herein may be independently combined and include “about” the quantity, range or ratio limit in question.

Also, it is understood that each and every feature of each aspect of the invention, e.g. the conjugates of the invention, may be considered to represent a preferred feature of each and every other said aspects of the invention.

### DETAILED DESCRIPTION OF THE INVENTION

In particular, the invention relates to conjugates of formula (1), (1a) or (1b):

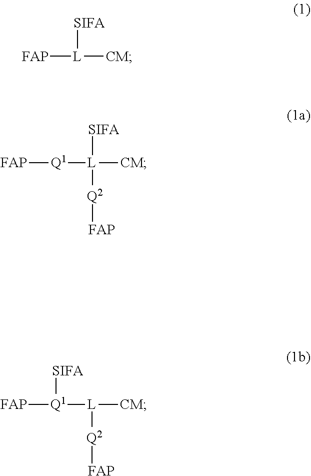

or a pharmaceutically or diagnostically acceptable salt or solvate thereof, wherein;
FAP represents a ligand which is capable of binding to Fibroblast activation protein (FAP) as defined herein;
L is an optionally substituted multivalent linker group as defined herein;
Q¹ and Q² in the conjugates of formulae 1a and 1b are optionally substituted linker groups which may be the same of different;
SIFA represents the silicon-fluoride acceptor (SIFA) moiety which comprises a covalent bond between a silicon and a fluorine atom and which SIFA moiety optionally include ¹⁸F; and
CM represents a chelating moiety, optionally containing a chelated nonradioactive or radioactive cation, as defined herein.

Conjugates of the invention may comprise a single FAP ligand (i.e. conjugates of formula (1)), two FAP ligands (i.e. formulae (1a) or (1b)) or three or more FAP ligands, which may be the same or different.

In some embodiments of the present invention, conjugates may comprise one or more ligands which are capable of binding to PSMA in addition to one or more of ligands which are capable of binding to FAP.

It is understood that collectively Q¹-L-Q² in the conjugates of formulae 1a and 1b together represent the multivalent linker group as defined herein.

The conjugate may be a conjugate of formula (4), (4a) or (4b):

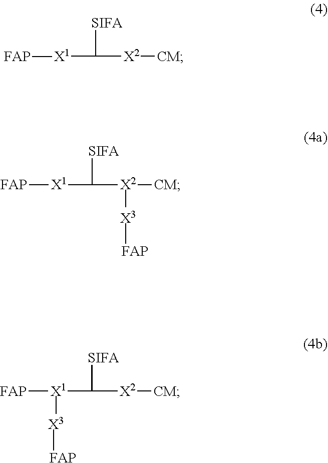

or a pharmaceutically or diagnostically acceptable salt or solvate thereof, wherein X¹, X² and X³ represent divalent linking groups, and where X¹, X² and X³ together with the groups to which they are attached comprise one or more amide bonds.

X¹ may be an optionally substituted 5-30 atom linker comprising 1 or more amide bonds. X¹ may be an optionally substituted 5-30 atom linker comprising 1 or more amide bonds, wherein the optional substituent is selected from —X³-FAP, CO₂H and CH₂OH. X¹ may be an optionally substituted 10-20 atom linker comprising 1 or more amide bonds. X¹ may be an optionally substituted 10-20 atom linker comprising 1 or more amide bonds, wherein the optional substituent is selected from —X³-FAP, CO₂H and CH₂OH.

X² may be an optionally substituted 1-30 atom linker comprising 1 or more amide bonds. X² may be an optionally substituted 1-10 atom linker comprising 1 or more amide bonds. X² may be an optionally substituted 1-5 atom linker comprising 1 or more amide bonds. In compounds of formula (4) or (4b), X² may also be —NH— or represent a bond.

X³ may be an optionally substituted 1-30 atom linker comprising 1 or more amide bonds. X³ may be an optionally substituted 5-30 atom linker comprising 1 or more amide bonds, wherein the optional substituent is selected from CO₂H and CH₂OH. X³ may be an optionally substituted 10-20 atom linker comprising 1 or more amide bonds. X³ may be an optionally substituted 10-20 atom linker comprising 1 or more amide bonds, wherein the optional substituent is selected from CO₂H and CH₂OH.

X¹ may be a group of formula:

—[(CR¹¹R¹²)ₙ(NHCO)ₘ(CONH)ₚ]_{q}—
wherein independently within each repeating unit:
   n is 1 to 10;
   m is 0 or 1;
   p is 0 or 1, where m and p cannot both be 1;
   q is 1 to 8; and
   R¹¹ and R¹² at each occurrence are independently selected from H, CO₂H and CH₂OH;
   and where one occurrence of R¹¹ and R¹² may be —X³-FAP.

X² may be a group of formula:

—[(CR¹¹R¹²)ₙ(NHCO)ₘ(CONH)ₚ]_{q}—
wherein independently within each repeating unit:
   n is 1 to 10;
   m is 0 or 1;
   p is 0 or 1, where m and p cannot both be 1;
   q is 1 to 8; and
   R¹¹ and R¹² at each occurrence are independently selected from H, CO₂H and CH₂OH;
   and where one occurrence of R¹¹ and R¹² may be —X³—FAP.

X³ may be a group of formula:

—[(CR¹¹R¹²)ₙ(NHCO)ₘ(CONH)ₚ]_{q}—
wherein independently within each repeating unit:
   n is 1to 10;
   m is 0 or 1;
   p is 0 or 1, where m and p cannot both be 1,
   q is 1 to 8; and
   R¹¹ and R¹² at each occurrence are independently selected from H, CO₂H and CH₂OH.

Independently within each repeating unit: n can be 1-10. n can be 1-5. n can be 1-3. n can be 1. n can be 2. n can be 3. n can be 4. n can be 5. n can be 6. n can be 7. n can be 8. n can be 9. n can be 10.

Independently within each repeating unit: m can be 0 and p can be 1. m can be 1 and p can be 0. m and p can both be 0.

q can be 1-8. q can be 1-5. q can be 1-3. q can be 1. q can be 2, q can be 3 q can be 4 q can be 5. q can be 6. q can be 7. q can be 8.

X¹ can be selected from:
—R¹³—NH—C(O)—R¹⁴—C(O)—NH—R¹⁵—NH—C(O)—; and
—R¹³—NH—C(O)—R¹⁴—NH—C(O)—R¹⁵—NH—C(O)—R¹⁶—NH—C(O)—;
   wherein R¹³, R¹⁴, R¹⁵ and R¹⁶ are independently C₁₋₁₀ alkyl, which alkyl groups may each be substituted by one or more substituents independently selected from —H, —OH, —OCH₃, —CH₂OH, —CO₂H, —CO₂CH₃, —NH₂, —CH₂NH₂ and —NHC(NH)NH₂; and where one of R¹³, R¹⁴, R¹⁵ and R¹⁶ may be substituted with —X³-FAP

X¹ can be selected from:
—CH(COOH)—R¹⁷—NH—C(O)—R¹⁸—C(O)—NH—R¹⁹—CH(COOH)—NH—C(O)—; and
—CH(COOH)—R¹⁷—NH—C(O)—R¹⁸—NH—C(O)—R¹⁹—NH—C(O)—CH(CH₂OH)—NH—C(O)—;
   wherein R¹⁷, R¹⁸ and R¹⁹ are independently C₁₋₆ alkyl.

X³ can be selected from:
—R¹³—NH—C(O)—R¹⁴—C(O)—NH—R¹⁵—NH—C(O)—; and
—R¹³—NH—C(O)—R¹⁴—NH—C(O)—R¹⁵—NH—C(O)—R¹⁶—NH—C(O)—;
   wherein R¹³, R¹⁴, R¹⁵ and R¹⁶ are independently C₁₋₁₀ alkyl, which alkyl groups may each be substituted by one or more substituents independently selected from —H, —OH, —OCH₃, —CH₂OH, —CO₂H, —CO₂CH₃, —NH₂, —CH₂NH₂ and —NHC(NH)NH₂; and where one of R¹³, R¹⁴, R¹⁵ and R¹⁶ may be substituted with —X³-FAP.

X² can be —NH—, —NH—C(O)—, —NH—C(O)—CH₂—, —NH—C(O)—CH₂CH₂— or —NH—C(O)—CH₂CH₂—CH(COOH)—; each of which may be substituted with —X³-FAP. In compounds of formula (4) or (4b) X² can be a bond.

X³ can be selected from:
—CH(COOH)—R¹⁷—NH—C(O)—R¹⁸—C(O)—NH—R¹⁹—CH(COOH)—NH—C(O)—; and
—CH(COOH)—R¹⁷—NH—C(O)—R¹⁸—NH—C(O)—R¹⁹—NH—C(O)—CH(CH₂OH)—NH—C(O)—:
   wherein R¹⁷, R¹⁸ and R¹⁹ are independently C₁₋₆ alkyl.

The conjugate may be a conjugate of formula (5), (5a), (5b), (5c) or (5d):

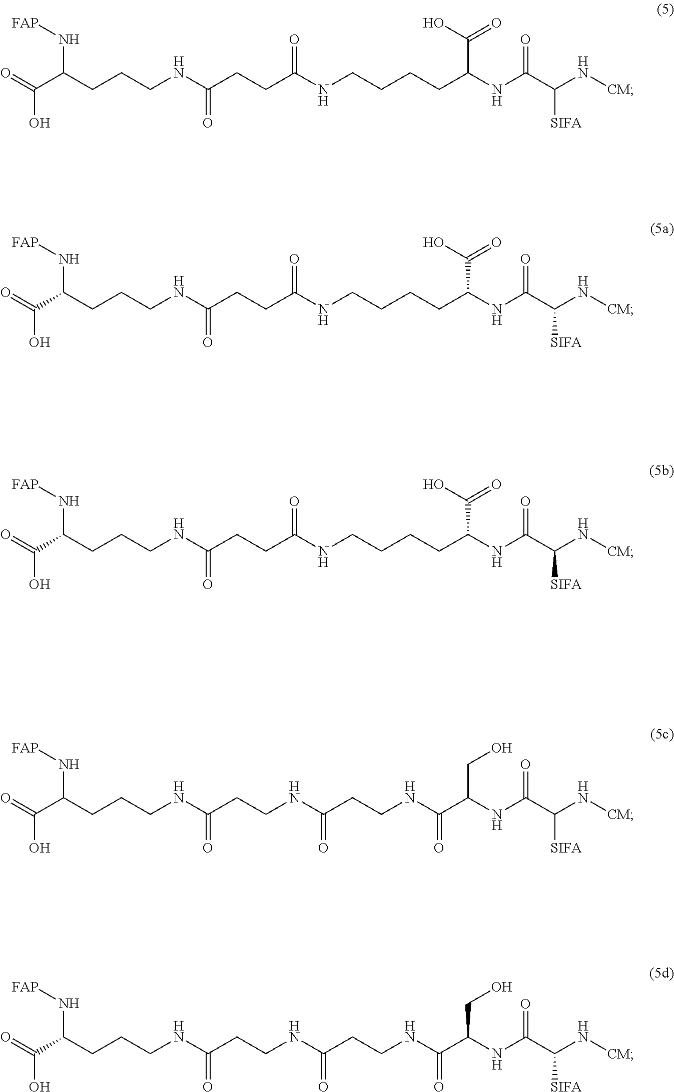

or a salt thereof, wherein FAP represents the ligand which is capable of binding to Fibroblast activation protein (FAP), SIFA represents the silicon-fluoride acceptor (SIFA) moiety and CM represents the chelating moiety; and wherein the linker linking FAP and CM may be optionally substituted with —X³-FAP at any available position.

The conjugate may be a conjugate of formula (5), (5a), (5b), (5c) or (5d):

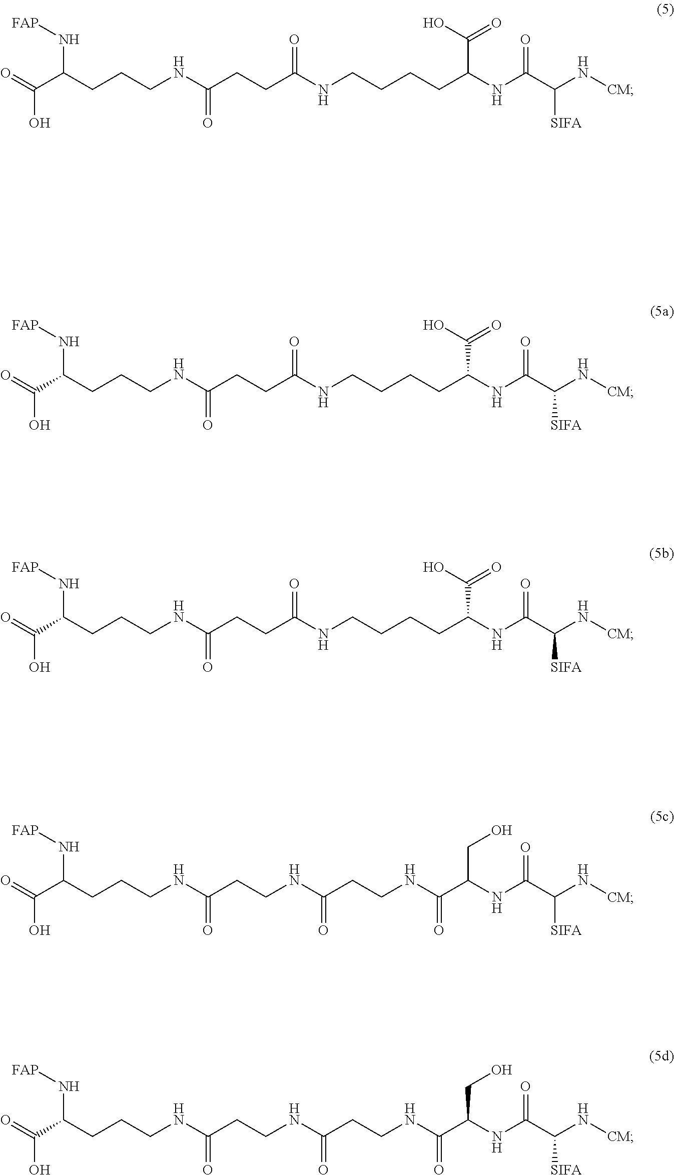

or a salt thereof, wherein FAP represents the ligand which is capable of binding to Fibroblast activation protein (FAP), SIFA represents the silicon-fluoride acceptor (SIFA) moiety and CM represents the chelating moiety.

### FAP Ligand

In the conjugates of the invention, the ligand which is capable of binding to Fibroblast Activation Protein (FAP) can be a functional group comprising a moiety which is capable of binding to FAP. The ligand which is capable of binding to Fibroblast Activation Protein (FAP) can comprise a substituted pyrrolidine ring. The ligand which is capable of binding to Fibroblast Activation Protein (FAP) can comprise a pyrrolidine ring substituted with CN and optionally one or more F atoms.

Compounds may include multiple FAP binding domains per conjugate. Thus compounds may include two or more ligands capable of binding to Fibroblast Activation Protein (FAP).

The ligand which is capable of binding to Fibroblast Activation Protein (FAP) can comprise a moiety of formula (2):

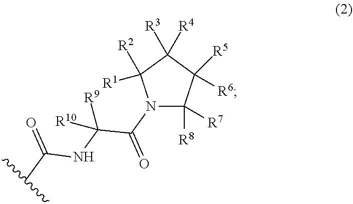

wherein, R¹, R², R³, R⁴, R⁵, R⁶, R⁷ and R⁸ are independently selected from H, OH, B(OH)₂, CO₂H, CN, halo, C₁₋₆ alkyl and —O—C₁₋₆ alkyl; and
R⁹ and R¹⁰ are independently H or C₁₋₆ alkyl.

The ligand which is capable of binding to Fibroblast Activation Protein (FAP) can comprise a moiety of formula (2a):

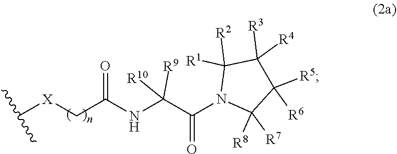

wherein, R¹, R², R³, R⁴, R⁵, R⁶, R⁷ and R⁸ are independently selected from H, OH, B(OH)₂, CO₂H, CN, halo, C₁₋₆ alkyl and —O—C₁₋₆ alkyl;
R⁹ and R¹⁰ are independently H or C₁₋₆ alkyl;
n is 0 to 3; and
X is a 5 to 10-membered N-containing monocyclic or bicyclic heterocycle which optionally further comprises 1, 2 or 3 heteroatoms selected from O, N and S and is optionally substituted with 1 to 3 substituents selected from C₁₋₆ alkyl, —O—C₁₋₆ alkyl, —S—C₁₋₆ alkyl and —NR²⁰R²¹, where R²⁰ and R²¹ are independently selected from H and C₁₋₆ alkyl.

In conjugates comprising a moiety of formula (2) or (2a), R¹, R², R³, R⁴, R⁵, R⁶, R⁷ and R⁸ can be independently selected from H, CN, and F. R¹ can be H, CN or F. R² can be H, CN or F. R³ can be H, CN or F. R⁴ can be H, CN or F. R⁵ can be H, CN or F. Re can be H, CN or F. R⁷ can be H, CN or F. R⁸ can be H, CN or F. Particular compounds include those where one of R⁷ and R⁸ is CN and the other is H, R³ and R⁴ are H or F, and R¹, R², R⁵ and R⁶ are H.

In conjugates comprising a moiety of formula (2) or (2a), R⁹ and R¹⁰ can be independently H or C₁₋₆ alkyl. R⁹ and R¹⁰ can be independently H or methyl.

In conjugates comprising a moiety of formula (2a), n can be 0 to 3. n can be 0. n can be 1. n can be 2. n can be 3.

In conjugates comprising a moiety of formula (2a), X can be selected from:

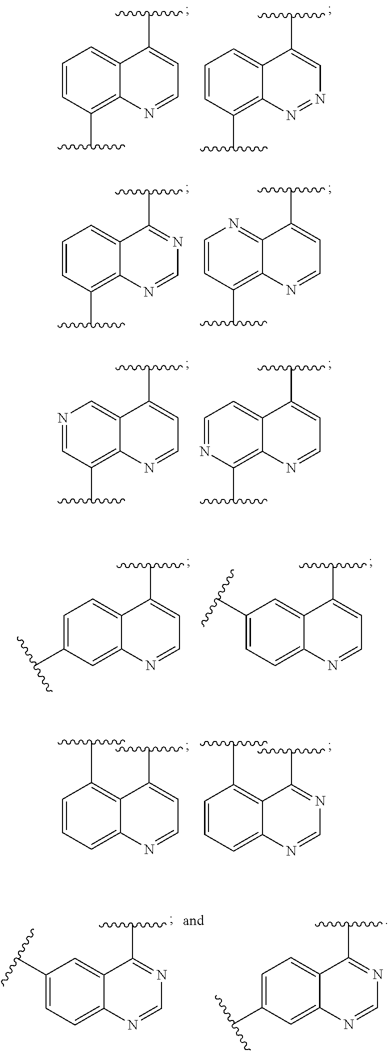

Particular conjugates may comprise a moiety which is selected from the group consisting of:

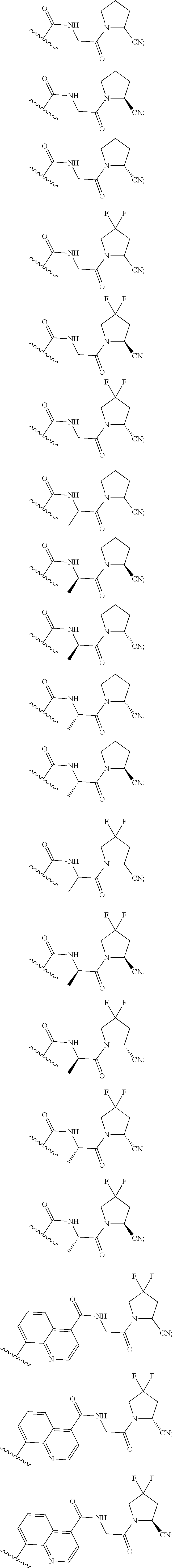

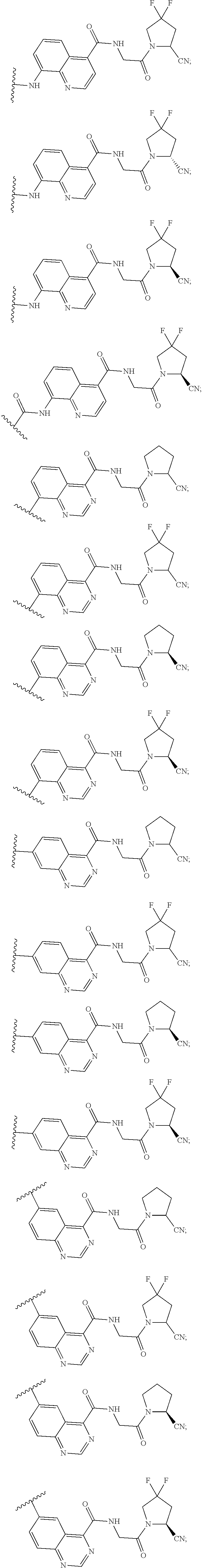

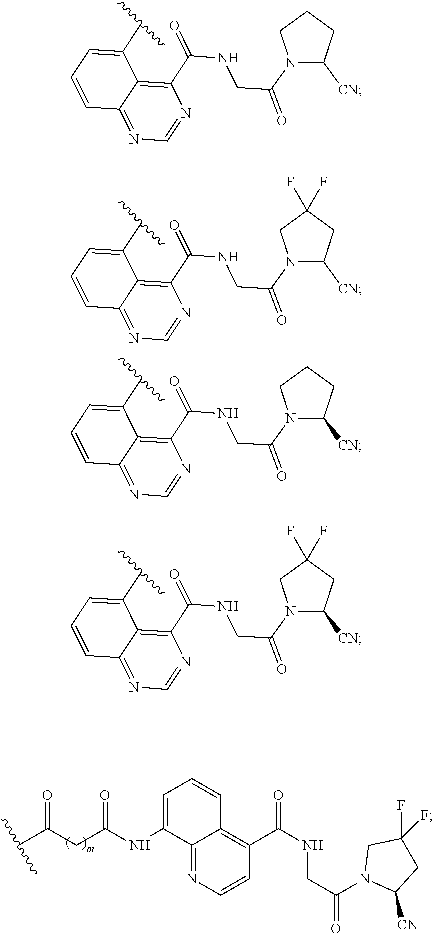

where m is 0 to 10.

In the conjugates of the invention, the ligand which is capable of binding to Fibroblast Activation Protein (FAP) may comprise a cyclic peptide moiety. The ligand may comprise a cyclic peptide moiety as described in WO2021/005125 or WO2021/005131.

Particular conjugates may comprise a moiety which is selected from the group consisting of:

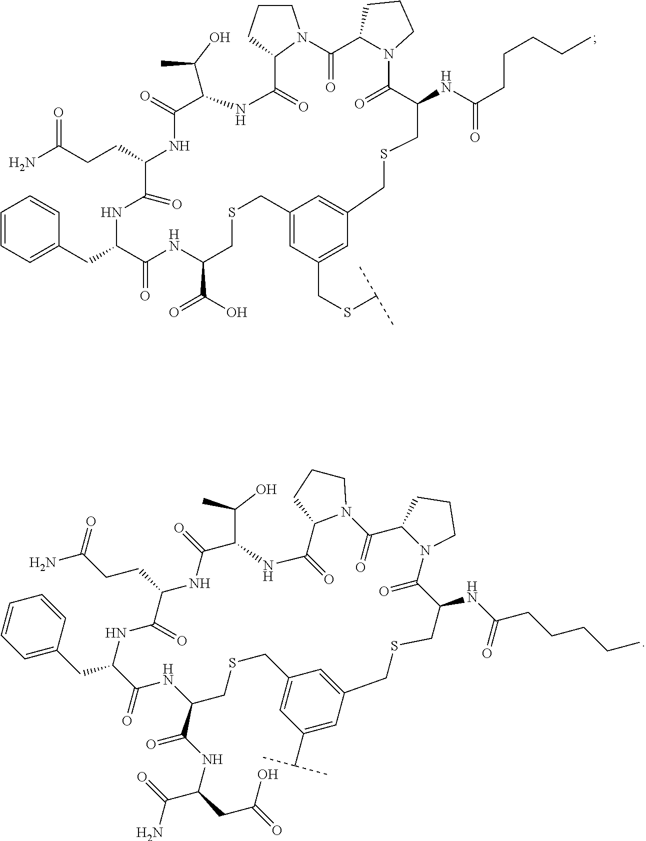

### PSMA Ligand

Some embodiments of the present invention comprise PSMA ligands that are disclosed in WO2019/020831. In some embodiments of the invention, the one or more ligands which are capable of binding to prostate-specific membrane antigen (PSMA ligand) comprises a structure represented by the following formula PSMA 1:

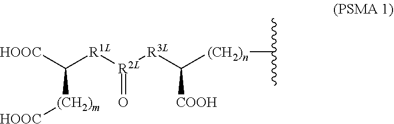

wherein m is an integer of 2 to 6, preferably 2 to 4, more preferably 2; n is an integer of 2 to 6, preferably 2 to 4, more preferably 2 or 3; R^{1L} is CH₂, NH or O, preferably NH; R^{3L} is CH₂, NH or O, preferably NH; R^{2L} is C or P(OH), preferably C; and wherein the ligand is attached to the remainder of the conjugate via the bond marked by [symbol] .

In some embodiments of the invention, the one or more ligands which are capable of binding to prostate-specific membrane antigen (PSMA ligand) comprises a structure represented by the following formula PSMA 2:

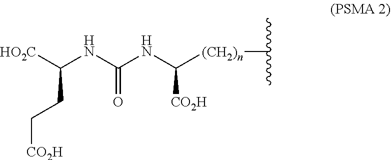

wherein n is an integer of 2 to 6; and wherein the ligand is attached to the remainder of the conjugate via the bond marked by [symbol] .

In some embodiments of the invention, the one or more ligands which are capable of binding to prostate-specific membrane antigen (PSMA ligand) comprises a structure represented by the following formula PSMA 3:

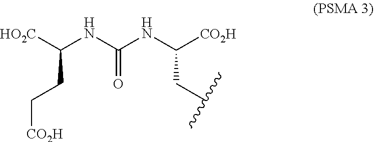 [SMILES: *C[C@H](NC(=O)N[C@@H](CCC(=O)O)C(=O)O)C(=O)O]

In some embodiments of the invention, the one or more ligands which are capable of binding to prostate-specific membrane antigen (PSMA ligand) comprises a structure represented by the following formula PSMA 4:

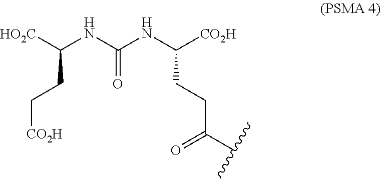 [SMILES: *C(=O)CC[C@H](NC(=O)N[C@@H](CCC(=O)O)C(=O)O)C(=O)O]

In some embodiments of the invention, the one or more ligands which are capable of binding to prostate-specific membrane antigen (PSMA ligand) comprises a structure represented by the following formula PSMA 5:

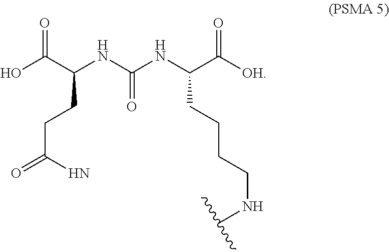 [SMILES: *NCCCC[C@H](NC(=O)N[C@@H](CCC(N)=O)C(=O)O)C(=O)O]

The conjugates of the invention comprise a silicon-fluoride acceptor (SIFA) moiety which comprises a covalent bond between a silicon and a fluorine atom. In the SIFA moiety the fluorine atom can be any known isotope of F or any combination thereof. In particular the fluorine atom of the SIFA moiety may be ¹⁹F or ¹⁸F. For diagnostic imaging and therapy, the fluorine atom of the SIFA moiety may be ¹⁸F. The ¹⁸F can be introduced by isotopic exchange with ¹⁹F.

Whilst certain ligands which are capable of binding to a disease-relevant target molecule may be cyclic peptides, such cyclic peptides are not chelating groups as envisaged herein, as the problem of the hydrophobic SIFA moiety is not solved in the absence of a further chelating moiety. Thus compounds of the invention require a hydrophilic chelating group in addition to the ligands which are capable of binding to a disease-relevant target molecule. The hydrophilic chelating group is required to reduce the hydrophobic nature of the compounds caused by the presence of the SIFA moiety.

### SIFA Moiety

The silicon-fluoride acceptor (SIFA) moiety may comprise the structure represented by formula (3):

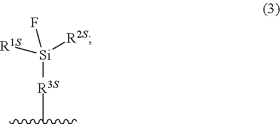

wherein: F is understood to encompass both ¹⁹F and ¹⁸F; R^{1S} and R^{2S} are independently a linear, branched or cyclic C₃ to C₁₀ alkyl group, preferably R^{1S} and R^{2S} are selected from isopropyl and tert-butyl, and are more preferably R^{1S} and R^{2S} are tert-butyl; R^{3S} is a C₁ to C₂₀ hydrocarbon group which may comprise one or more aromatic and one or more aliphatic units and/or up to 3 heteroatoms selected from O and S, preferably R^{3S} is a C₆ to C₁₀ hydrocarbon group which comprises an aromatic ring and which may comprise one or more aliphatic units; more preferably R^{3S} is a phenyl ring, and most preferably, R^{3S} is a phenyl ring wherein the Si-containing substituent and the bond marked by [symbol] are in a para-position, and wherein the SIFA moiety is attached to the remainder of the conjugate via the bond marked by [symbol] .

The silicon-fluoride acceptor (SIFA) moiety may comprise the structure represented by formula (3):

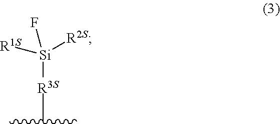

wherein: F is understood to encompass both ¹⁹F and ¹⁸F; R^{1S} and R^{2S} are independently a linear, branched or cyclic C₃ to C₁₀ alkyl group; R^{3S} is a C₁ to C₂₀ hydrocarbon group comprising one or more aromatic and/or aliphatic units and/or up to 3 heteroatoms selected from O and S;
and wherein the SIFA moiety is attached to the remainder of the conjugate via the bond marked by [symbol] .

In conjugates comprising the SIFA moiety of formula (3), R^{1S} and R^{2S} can be independently selected from a linear or branched C₁₋₆ alkyl group or a C₃₋₆ cycloalkyl group. R^{1S} and R^{2S} can be independently selected from methyl, ethyl, n-propyl, isopropyl, n-butyl, t-butyl, sec-butyl, cyclopropyl, cyclobutyl, cyclopentyl or cyclohexyl. R^{1S} and R^{2S} can be methyl. R^{1S} and R^{2S} can be isopropyl. R^{1S} and R^{2S} can be t-butyl. R^{1S} can be selected from methyl, ethyl, n-propyl, isopropyl, n-butyl, t-butyl, sec-butyl, cyclopropyl, cyclobutyl, cyclopentyl or cyclohexyl. R^{1S} can be methyl. R^{1S} can be isopropyl. R^{1S} can be t-butyl. R^{2S} can be selected from methyl, ethyl, n-propyl, isopropyl, n-butyl, t-butyl, sec-butyl, cyclopropyl, cyclobutyl, cyclopentyl or cyclohexyl. R^{2S} can be methyl. R^{2S} can be isopropyl. R^{2S} can be t-butyl.

In conjugates comprising the SIFA moiety of formula (3), R^{3S} is a C₁ to C₂₀ hydrocarbon group comprising one or more aromatic and/or aliphatic units and/or up to 3 heteroatoms selected from O and S. R^{3S} may be a phenyl ring. R^{3S} may be:

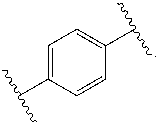 [SMILES: *c1ccc(*)cc1]

The silicon-fluoride acceptor (SIFA) moiety may comprise the structure represented by formula (3a) or (3b):

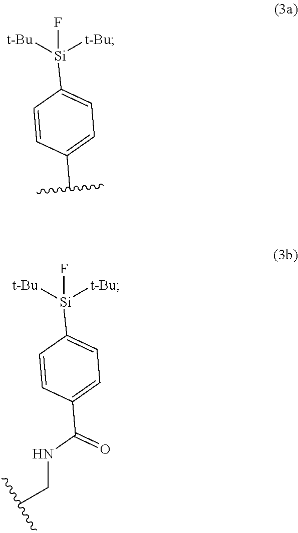 [SMILES: *c1ccc([Si](F)(C(C)(C)C)C(C)(C)C)cc1 ; *CNC(=O)c1ccc([Si](F)(C(C)(C)C)C(C)(C)C)cc1]

wherein t-Bu indicates a tert-butyl group and F is understood to encompass both ¹⁹F and ¹⁸F.

### Chelating Moiety

In the conjugates herein, a preferred chelating moiety (CM) comprises at least one of the following (i), (ii) or (iii):
(i) A macrocyclic ring structure with 8 to 20 ring atoms of which 2 or more, more preferably 3 or more, are selected from oxygen atoms or nitrogen atoms. Preferably, 6 or less ring atoms are selected from oxygen atoms or nitrogen atoms. Especially preferred is that 3 or 4 ring atoms are nitrogen atoms or oxygen atoms. Among the oxygen and nitrogen atoms, preference is given to the nitrogen atoms. In combination with the macrocyclic ring structure, the preferred chelating group may comprise 2 or more, such as 2 to 6, preferably 2 to 4, carboxyl groups and/or hydroxyl groups. Among the carboxyl groups and the hydroxyl groups, preference is given to the carboxyl groups.
(ii) An acyclic, open chain chelating structure with 8 to 20 main chain (back bone) atoms of which 2 or more, more preferably 3 or more are heteroatoms selected from oxygen atoms or nitrogen atoms. Preferably, 6 or less back bone atoms are selected from oxygen atoms or nitrogen atoms. Among the oxygen and nitrogen atoms, preference is given to the nitrogen atoms. More preferably, the open chain chelating structure is a structure which comprises a combination of 2 or more, more preferably 3 or more heteroatoms selected from oxygen atoms or nitrogen atoms, and 2 or more, such as 2 to 6, preferably 2 to 4, carboxyl groups and/or hydroxyl groups. Among the carboxyl groups and the hydroxyl groups, preference is given to the carboxyl groups.
(iii) A branched chelating structure containing a quaternary carbon atom. Preferably the quaternary carbon atom is substituted with 3 identical chelating groups in addition to the remainder of the conjugate. The substituted chelating groups can comprise an amide. The substituted chelating groups can comprise an aromatic group. The substituted chelating groups can comprise a hydroxypyridinone.

The chelating moiety may comprise at least one of:
(i) a macrocyclic ring structure with 8 to 20 ring atoms of which 2 or more are heteroatoms selected from oxygen atoms and nitrogen atoms,
(ii) an acyclic, open chain chelating structure with 8 to 20 main chain atoms of which 2 or more are heteroatoms selected from oxygen atoms and nitrogen atoms, or
(iii) a branched chelating structure containing a quaternary carbon atom.

The chelating moiety (CM) may be selected from bis(carboxymethyl)-1,4,8,11-tetraazabicyclo[6.6.2]hexadecane (CBTE2a), cyclohexyl-1,2-diaminetetraacetic acid (CDTA), 4-(1,4,8,11-tetraazacyclotetradec-1-yl)-methylbenzoic acid (CPTA), N′-[5-[acetyl(hydroxy)amino]pentyl]-N-[5-[[4-[5-aminopentyl-(hydroxy)amino]-4-oxobutanoyl]amino]pentyl]-N-hydroxybutandiamide (DFO), 4,11-bis(carboxymethyl)-1,4,8,11-tetraazabicyclo[6.6.2]hexadecane (DO2A) 1,4,7,10-tetracyclododecan-N,N′,N″,N′″-tetraacetic acid (DOTA), α-(2-carboxyethyl)-1,4,7,10-tetraazacyclododecane-1,4,7,10-tetraacetic acid (DOTAGA), 1,4,7,10 tetraazacyclododecane N, N′, N″, N′″ 1,4,7,10-tetra(methylene) phosphonic acid (DOTMP), N,N′-dipyridoxylethylendiamine-N,N′-diacetate-5,5′-bis(phosphat) (DPDP), diethylene triamine N,N′,N″ penta(methylene) phosphonic acid (DTMP), diethylenetriaminepentaacetic acid (DTPA), ethylenediamine-N,N′-tetraacetic acid (EDTA), ethyleneglycol-O,O-bis(2-aminoethyl)-N,N,N′,N′-tetraacetic acid (EGTA), N,N-bis(hydroxybenzyl)-ethylenediamine-N,N′-diacetic acid (HBED), hydroxyethyldiaminetriacetic acid (HEDTA), 1-(p-nitrobenzyl)-1,4,7,10-tetraazacyclodecan-4,7,10-triacetate (HP-DOA3), 6-hydrazinyl-N-methylpyridine-3-carboxamide (HYNIC), tetra 3-hydroxy-N-methyl-2-pyridinone chelators (4-((4-(3-(bis(2-(3-hydroxy-1-methyl-2-oxo-1,2-dihydropyndine-4-carboxamido)ethyl)amino)-2-((bis(2-(3-hydroxy-1-methyl-2-oxo-1,2-dihydropyridine-4-carboxamido)ethyl)amino)methyl)propyl)phenyl)amino)-4-oxobutanoic acid), abbreviated as Me-3,2-HOPO, 1,4,7-triazacyclononan-1-succinic acid-4,7-di acetic acid (NODASA), 1-(1-carboxy-3-carboxypropyl)-4,7-(carbooxy)-1:4,7-triazacyclononane (NODAGA), 1,4,7-triazacyclononanetriacetic acid (NOTA), 4:11-bis(carboxymethyl)-1,4,8,11-tetraazabicyclo[6.6.2]hexadecane (TE2A), 1,4,8,11-tetraazacyclododecane-1,4,8,11-tetraacetic acid (TETA), tris(hydroxypyridinone) (THP), terpyridin-bis(methyleneamintetraacetic acid (TMT), 1,4,7-triazacyclononane-1,4,7-tris[methylene(2-carboxyethyl)phosphinic acid] (TRAP), 1,4,7,10-tetraazacyclotridecan-N,N′,N″,N′″-tetraacetic acid (TRITA), 3-[[4,7-bis[[2-carboxyethyl(hydroxy)phosphoryl]methyl]-1,4,7-triazonan-1-yl]methyl-hydroxy-phosphoryl]propanoic acid, triethylenetetraaminehexaacetic acid (TTHA), and 1,4-bis(carboxymethyl)-6-[bis(carboxymethyl)]amino-6-methylperhydro-1,4-diazepine (AAZTA).

Particular chelating moieties (CM) are shown below:

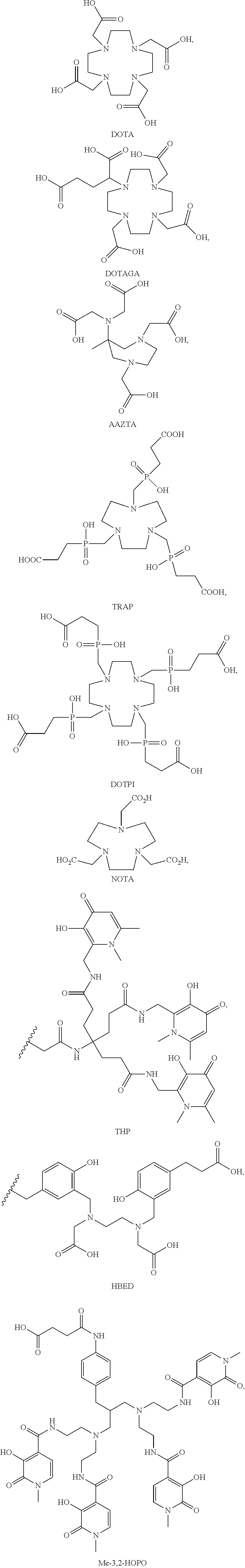

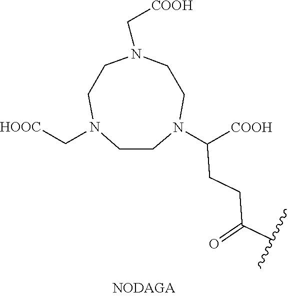 [SMILES: *C(=O)CCC(C(=O)O)N1CCN(CC(=O)O)CCN(CC(=O)O)CC1]

Particular chelating moieties (CM) include:

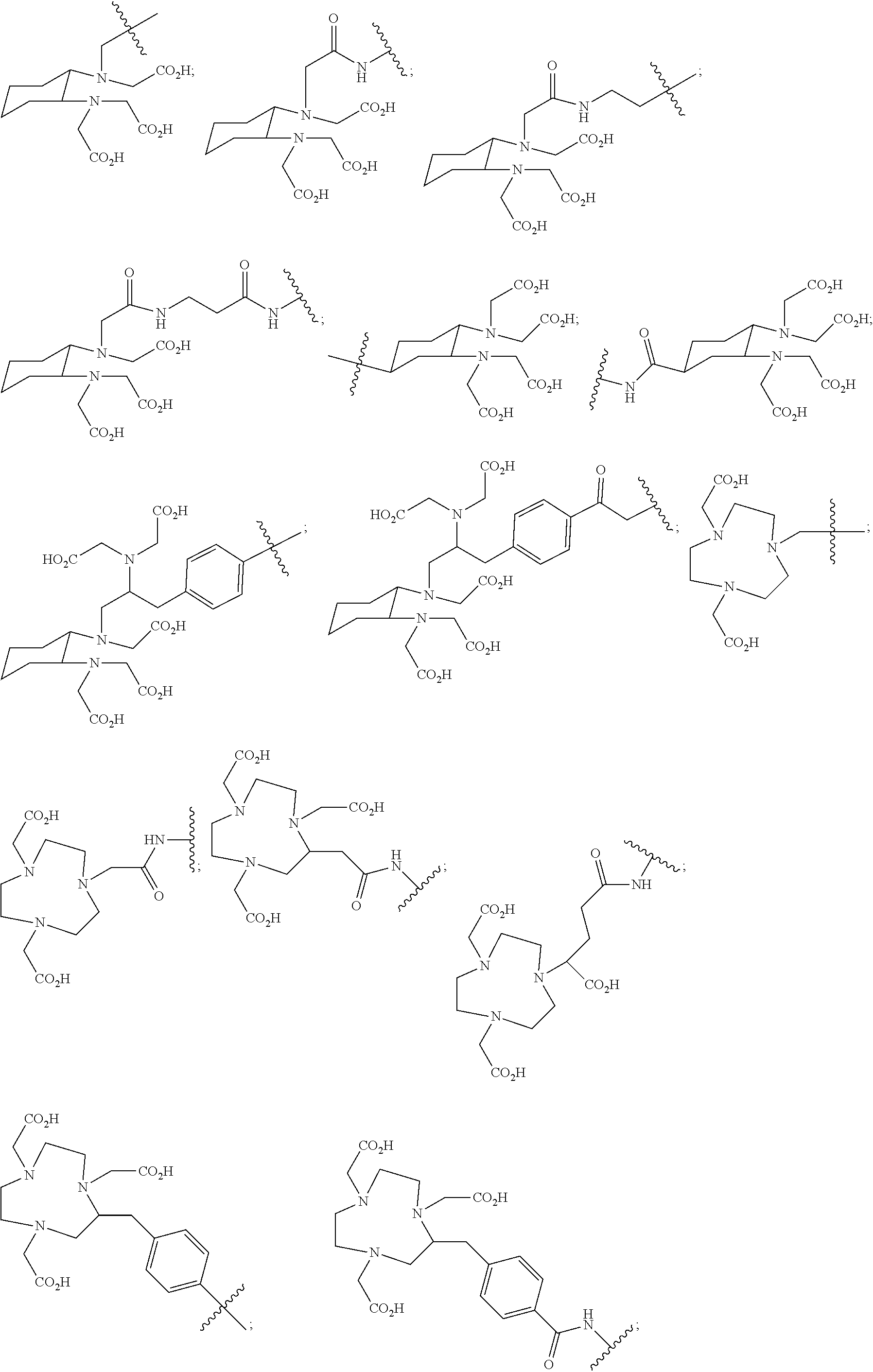

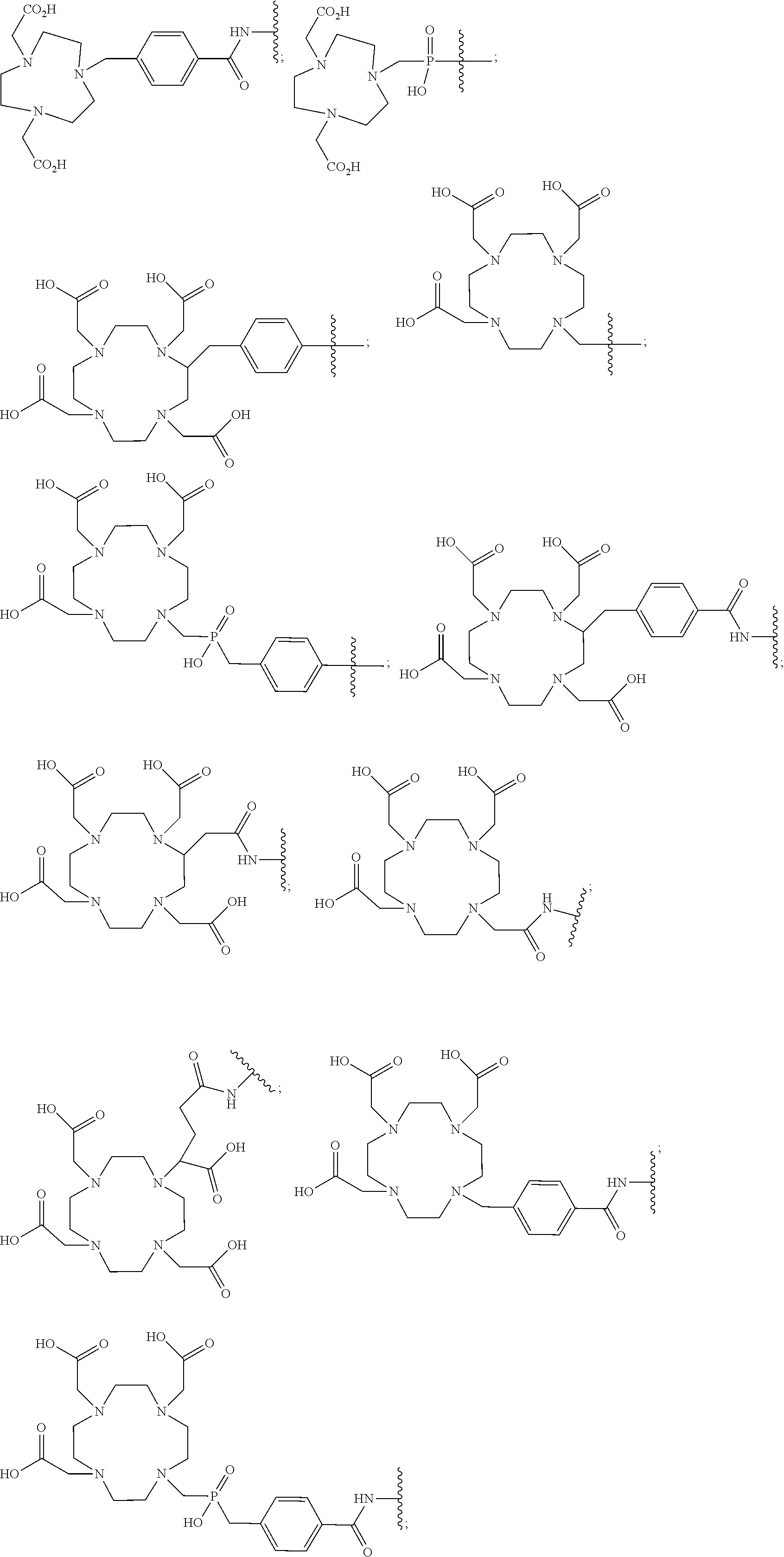

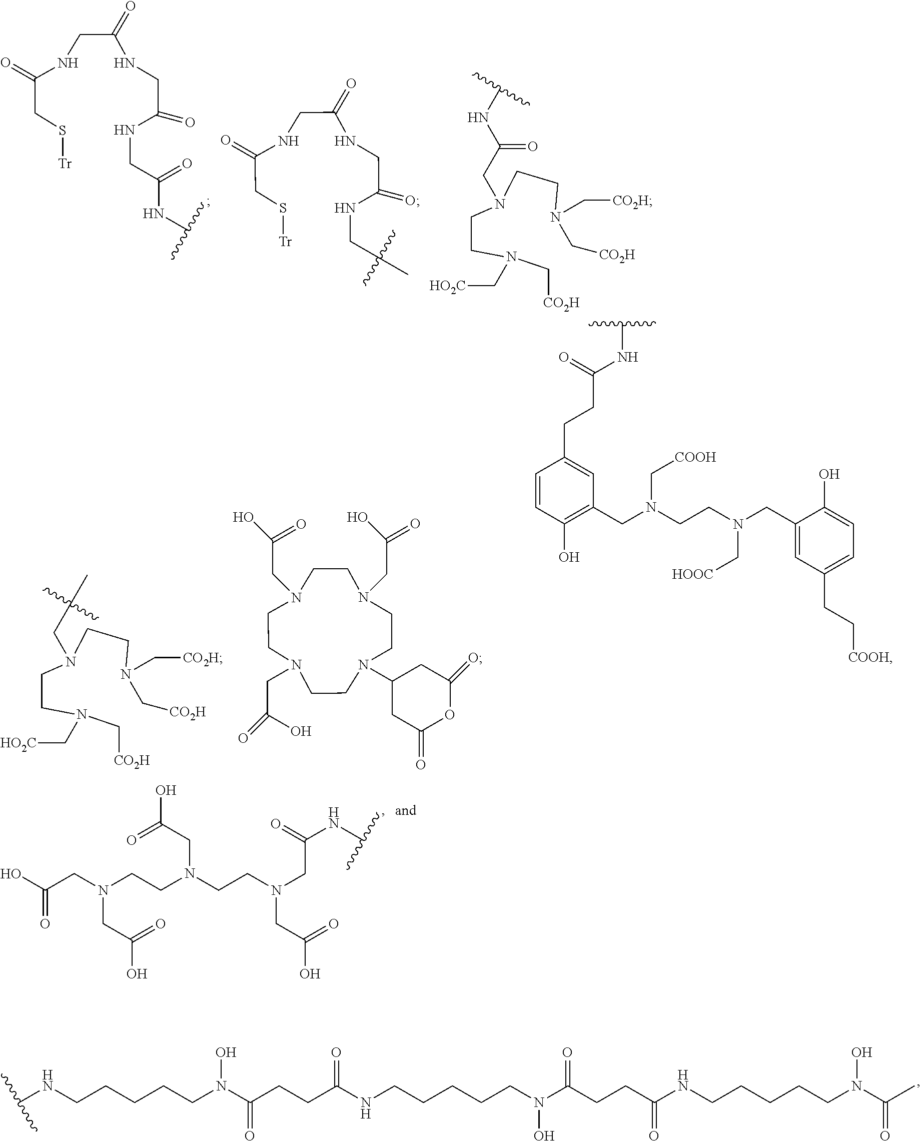

Particular chelating moieties (CM) include:

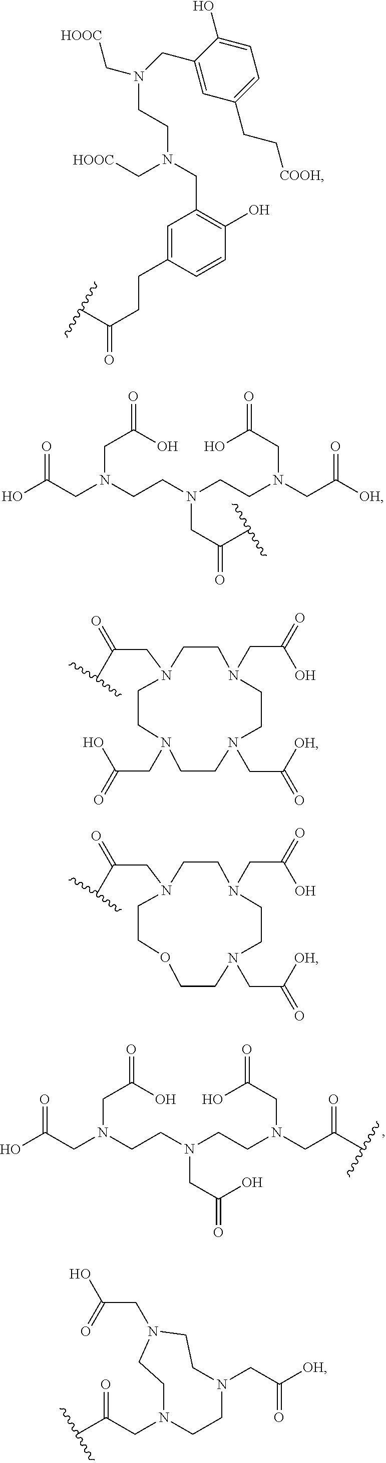

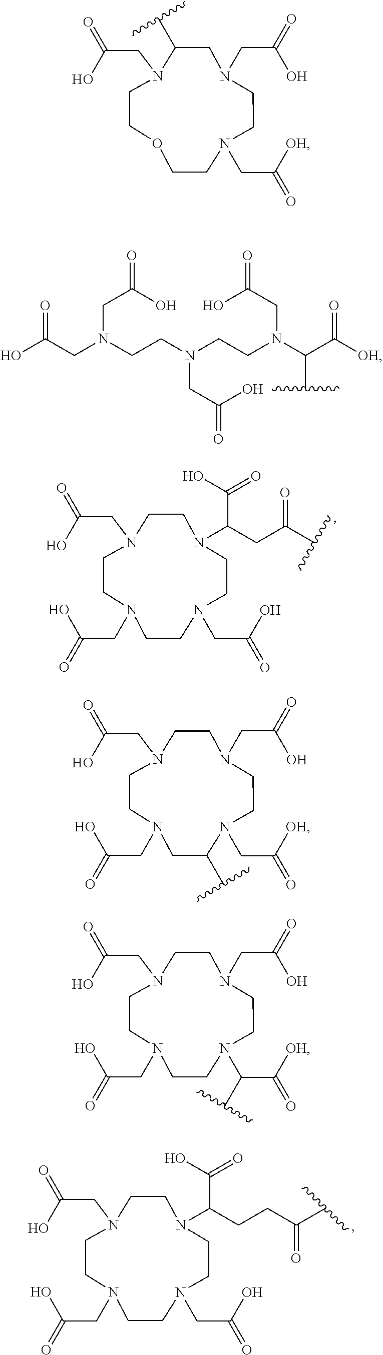

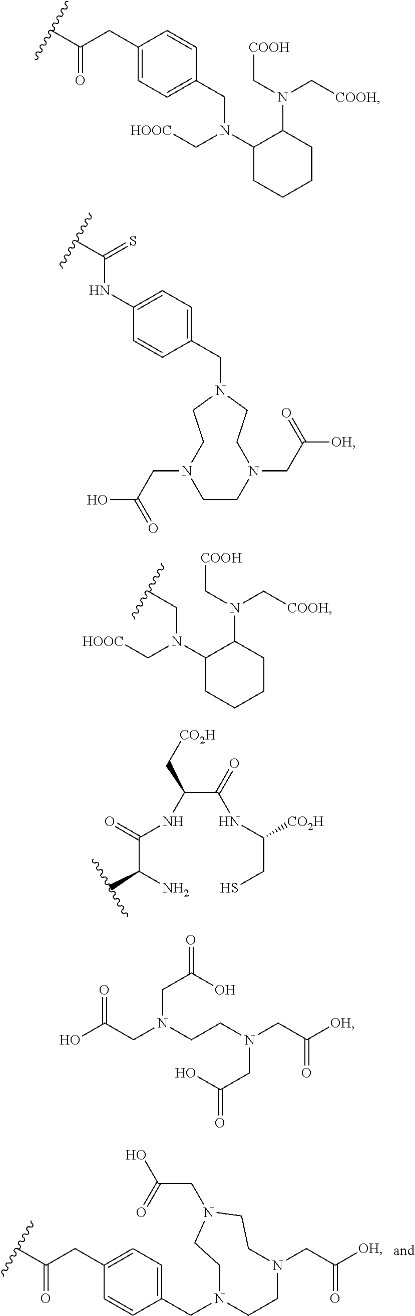 [SMILES: *C(=O)Cc1ccc(CN(CC(=O)O)C2CCCCC2N(CC(=O)O)CC(=O)O)cc1 ; *C(=S)Nc1ccc(CN2CCN(CC(=O)O)CCN(CC(=O)O)CC2)cc1 ; *CN(CC(=O)O)C1CCCCC1N(CC(=O)O)CC(=O)O ; *[C@H](N)C(=O)N[C@@H](CC(=O)O)C(=O)N[C@@H](CS)C(=O)O ; O=C(O)CN(CCN(CC(=O)O)CC(=O)O)CC(=O)O ; *C(=O)Cc1ccc(CN2CCN(CC(=O)O)CCN(CC(=O)O)CC2)cc1]

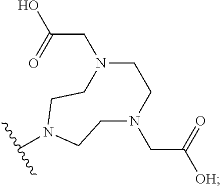 [SMILES: *N1CCN(CC(=O)O)CCN(CC(=O)O)CC1]

Among the above exemplary chelating agents, particular preference is given to a chelating moiety selected from TRAP, DOTA and DOTAGA.

The chelating moiety (CM) may be 1,4,7,10-tetracyclododecan-N,N′,N″,N′″-tetraacetic acid (DOTA):

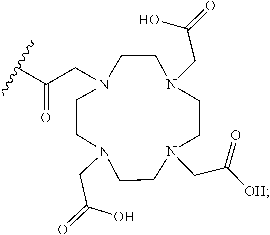 [SMILES: *C(=O)CN1CCN(CC(=O)O)CCN(CC(=O)O)CCN(CC(=O)O)CC1]

or α-(2-carboxyethyl)-1,4,7,10-tetraazacyclododecane-1,4,7,10-tetraacetic acid (DOTAGA):

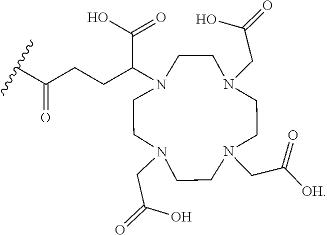 [SMILES: *C(=O)CCC(C(=O)O)N1CCN(CC(=O)O)CCN(CC(=O)O)CCN(CC(=O)O)CC1]

The chelating moiety (CM) may be selected from:

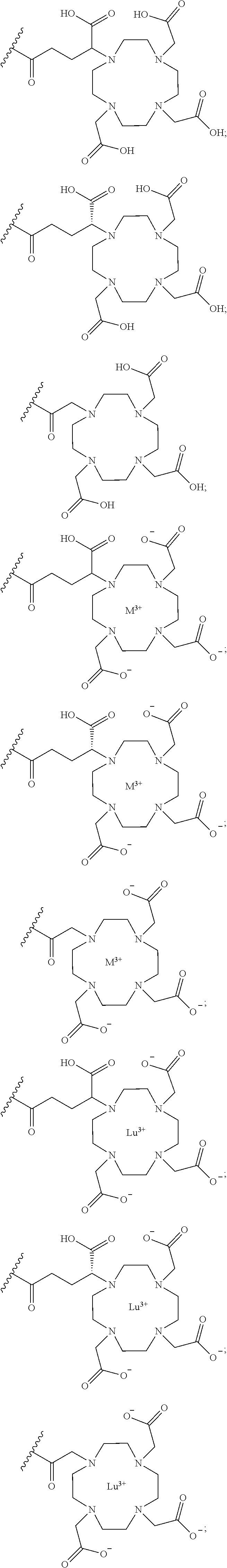

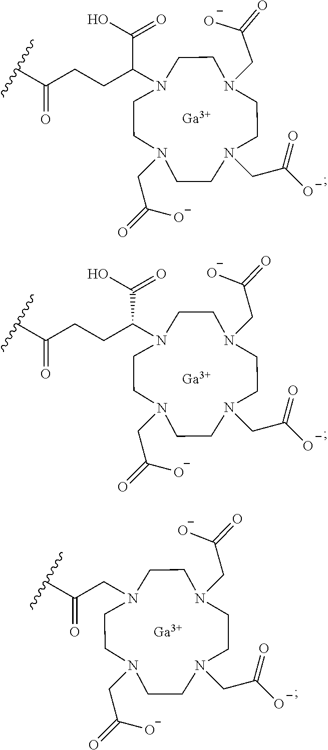

wherein M represents a chelated metal cation.

Metal- or cation-chelating macrocyclic and acyclic compounds are well-known in the art and available from a number of manufacturers. While the chelating moiety in accordance with the present invention is not particularly limited, it is understood that numerous moieties can be used in an off-the-shelf manner by a skilled person without further ado.

The chelating moiety may comprise a chelated cation which may be radioactive or non-radioactive, preferably a chelated metal cation which may be radioactive or non-radioactive. The chelating moiety may comprise a chelated cation which is radioactive. The chelating moiety may comprise a chelated cation which is non-radioactive.

Especially preferred is that CM represents a chelating moiety selected from DOTA and DOTAGA bound with one of its carboxylic groups via an amide bond to the remainder of the conjugate.

Preferred examples of cations that may be chelated by the chelating group are the radioactive or non-radioactive cations of Sc, Cr, Mn, Co, Fe, Ni, Cu, Ga, Zr, Y, Tc, Ru, Rh, Pd, Ag, In, Sn, Te, Pr, Pm, Tb, Sm, Gd, Tb, Ho, Dy, Er, Yb, Tm, Lu, Re, Pt, Hg, Au, Pb, Bi, Ra, Ac, Th; more preferably the cations of Sc, Cu, Ga, Y, In, Tb, Ho, Lu, Re, Pb, Bi, Ac, Th and Er. The cation may be Ga. The cation may be Lu.

The chelating moiety may contain a chelated cation or cationic species selected from the cations of ⁴³Sc, ⁴⁴Sc, ⁴⁷Sc, ⁵¹Cr, ^{52m}Mn, ⁵⁸Co, ⁵²Fe, ⁵⁸Ni, ⁵⁷Ni, ⁶¹Cu, ⁶²Cu, ⁶⁴Cu, ⁶⁷Cu, ⁶⁶Ga, ⁶⁷Ga ⁶⁸Ga, ⁸⁹Zr, ⁹⁰Y, ⁸⁹Y, <Tc, ^{99m}TC, ⁹⁷Ru, ¹⁰⁵Rh, ¹⁰⁹Pd, ¹¹¹Ag, ^{110m}In, ¹¹¹In, ^{113m}In, ^{114m}In, ^{117m}Sn, ¹²¹Sn, ¹²⁷Te, ¹⁴²Pr, ¹⁴³Pr, ¹⁴⁹Pm, ¹⁵¹Pm, ¹⁴⁹Tb, ¹⁵²Tb, ¹⁵⁵Tb, ¹⁶¹Tb, ¹⁵³Sm, ¹⁵⁷Gd, ¹⁸¹Tb, ¹⁶⁶Ho, ¹⁶⁵Dy, ¹⁶⁹Er, ¹⁶⁹Yb, ¹⁷⁵Yb, ¹⁷²Tm, ¹⁷⁷Lu, ¹⁸⁶Re, ¹⁸⁸Re, ¹⁹¹Pt, ¹⁹⁷Hg, ¹⁹⁸Au, ¹⁹⁹Au, ²¹²Pb, ²⁰³Pb, ²¹¹Al, ²¹²Bi, ²¹³Bi, ²²³Ra, ²²⁵Ac, ²²⁷Th, a cationic molecule comprising ¹⁸F or ²¹¹At, or a cation such as ¹⁸F—[AlF]²⁺; more preferably the cations of ⁴⁴Sc, ⁴⁷Sc, ⁶¹Cu, ⁶⁴Cu, ⁶⁷Cu, ⁶⁸Ga, ⁹⁰Y, ¹¹¹In, ¹⁶¹Tb, ¹⁶⁶Ho, ¹⁷⁷Lu, ¹⁸⁸Re, ²¹²Pb, ²¹²Bi, ²¹³Bi, ²²⁵Ac, and ²²⁷Th or a cationic molecule comprising ¹⁸F.

The chelating moiety may contain a chelated cation selected from the cations of ⁴³Sc, ⁴⁴Sc, ⁴⁷Sc, ⁶¹Cu, ⁶⁴Cu, ⁶⁷Cu, ⁶⁷Ga, ⁶⁸Ga, ⁹⁰Y, ¹¹¹in, ¹⁴⁹Tb, ¹⁵²Tb, ¹⁵⁵Tb, ¹⁶¹Tb, ¹⁶⁶Ho, ¹⁷⁷Lu, ¹⁸⁶Re, ¹⁸⁸Re, ²¹²Pb, ²¹²Bi, ²¹³Bi, ²²⁵Ac, and ²²⁷Th or a cationic molecule comprising ¹⁸F. The chelating moiety may contain a chelated cation selected from the cations of ⁶⁸Ga or ¹⁷⁷Lu. The chelating moiety may contain a chelated ⁶⁸Ga cation. The chelating moiety may contain a chelated ¹⁷⁷Lu cation. M may be selected from the cations of ⁴³Sc, ⁴⁴Sc, ⁴⁷Sc, ⁶¹Cu, ⁶⁴Cu, ⁶⁷Cu, ⁶⁷Ga, ⁶⁸Ga, ⁹⁰Y, ¹¹¹In, ¹⁴⁹Tb, ¹⁵²Tb, ¹⁵⁵Tb, ¹⁶¹Tb, ¹⁶⁶Ho, ¹⁷⁷Lu, ¹⁸⁶Re, ¹⁸⁸Re, ²¹²Pb, ²¹²Bi, ²¹³Bi, ²²⁵Ac, and ²²⁷Th. M may be selected from the cations of ⁶⁸Ga, ⁶⁴Cu, ¹⁷⁷Lu, ⁹⁰Y and ²²⁵Ac. M may be selected from the cations of ⁶⁸Ga and ¹⁷⁷Lu. M may be a chelated ⁶⁸Ga cation. M may be a chelated ¹⁷⁷Lu cation. M may be a chelated ⁶⁴Cu cation. M may be a chelated ⁹⁰Y cation. M may be a chelated ²²⁵Ac cation.

In the compounds herein, the chelated nonradioactive or radioactive cation of the chelating moiety may be chelated to one or more COO⁻ groups. The chelated nonradioactive or radioactive cation of the chelating moiety may be chelated to one or more N atoms. The chelated nonradioactive or radioactive cation of the chelating moiety may be chelated to one or more N atoms or one or more COO⁻ groups. The chelated nonradioactive or radioactive cation of the chelating moiety may be chelated to one or more N atoms and one or more COO⁻ groups. In the structures provided herein, where chelated nonradioactive or radioactive cations are shown, the groups to which they are chelated are merely representatively shown. For example, disclosure of a compound comprising the chelating moiety shown below includes within its scope all complexes or modes of chelation that are chemically possible between the Ga³⁺ cation and the conjugate as a whole:

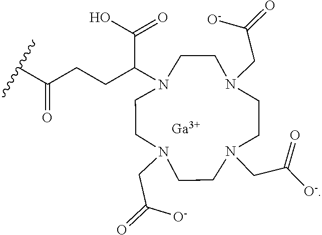

A key aspect of the invention is the combination, within a single molecule, of a silicon fluoride acceptor and a chelating group (chelator) or a chelate. These two structural elements, SIFA and the chelator, exhibit a spatial proximity. Preferably, the shortest distance between two atoms of the two elements is less or equal 25 Å, more preferably less than 20 Å and even more preferably less than 15 Å. Alternatively or in addition, it is preferred that not more than 25 covalent bonds separate an atom of the SIFA moiety and an atom the chelator, preferably not more than 20 chemical bonds and even more preferably not more than 15 chemical bonds.

Suitably, the cation is a radioactive or non-radioactive cation. It is preferably a radioactive or non-radioactive metal cation, and more preferably a radioactive metal cation. Examples are given further below.

As a consequence, conjugates fall under the terms of the first aspect which are radioactively labelled at both the SIFA moiety and the chelating group, molecules which are radioactive labelled at only one of the two sides, as well as molecules which are not radiolabelled at all. In the latter case, the chelating group may be either a complex of a cold (non-radioactive) ion or may devoid of any ion.

The placement of the silicone fluoride acceptor in the neighbourhood of a hydrophilic chelator such as, but not limited to, DOTAGA or DOTA, may shield or compensate efficiently the lipophilicity of the SIFA moiety to an extent which shifts the overall hydrophobicity of the radio-therapeutic or -diagnostic compound in a range which renders the compound suitable for in-vivo administration.

In addition, the combination of the use of a chelator and an isotopic exchange on SIFA by means of ¹⁸F-fluoride also results in “paired” diagnostic tracers that can either be used as [¹⁸F][^{nat}Ion]tracers at centers with onsite cyclotron or centers that obtain ¹⁸F-fluoride by shipment from cyclotron centers, whereas in centers, that do not have access to ¹⁸F-fluoride but have access to radioisotope generators, such as a Ge-68/Ga-68 generator, the corresponding versions, e.g. [^{nat}F][⁶⁸Ga]tracers can be used.

Importantly, in both cases, the chemically identical radiopharmaceutical is injected, and thus no differences in the in vivo behavior are expected. Whereas currently, due to chemical differences, the clinical data of a ¹⁸F-labelled compound provided by a patient cohort at one site cannot be directly compared with the clinical data of a ⁶⁸Ga-analogue provided by another group at another site, radiopharmaceuticals and/or diagnostics according to the invention can be directly compared and thus will allow to link such data (e.g. data from a center in Europe working with F-18 and another center in India working with Ga-68). Furthermore, when suitably selected, the chelate can also be used for labelling with a therapeutic isotope, such as the beta-emitting isotopes Lu-177, Y-90, or the alpha emitting isotope Ac-225, thus allowing to expand the concept of “paired” tracers to bridge diagnostic ([¹⁸F][^{nat}Lu]tracers) and therapeutic radiopharmaceuticals ([^{nat}F][¹⁷⁷Lu].

Also provided is a pharmaceutical imaging composition comprising or consisting of one or more conjugates of the invention as disclosed herein.

Also provided is a diagnostic composition comprising or consisting of one or more conjugates of the invention as disclosed herein.

Also provided is a therapeutic composition comprising or consisting of one or more conjugates of the invention as disclosed herein.

The pharmaceutical composition may further comprise pharmaceutically acceptable carriers, excipients and/or diluents. Examples of suitable pharmaceutical carriers, excipients and/or diluents are well known in the art and include phosphate buffered saline solutions, water, emulsions, such as oil/water emulsions, various types of wetting agents, sterile solutions etc. Compositions comprising such carriers can be formulated by well-known conventional methods. These pharmaceutical compositions can be administered to the subject at a suitable dose. Administration of the suitable compositions may be effected in different ways, e.g., by intravenous, intraperitoneal, subcutaneous, intramuscular, topical, intradermal, intranasal or intrabronchial administration. It is particularly preferred that said administration is carried out by injection and/or delivery, e.g., to a site in the pancreas or into a brain artery or directly into brain tissue. The compositions may also be administered directly to the target site, e.g., by biolistic delivery to an external or internal target site, like the pancreas or brain. The dosage regimen will be determined by the attending physician and clinical factors. As is well known in the medical arts, dosages for any one patient depends upon many factors, including the patient's size, body surface area, age, the particular compound to be administered, sex, time and route of administration, general health, and other drugs being administered concurrently. Pharmaceutically active matter may be present in an effective therapeutic amount, which may be between 0.1 ng and 10 mg/kg body weight per dose; however, doses below or above this exemplary range are envisioned, especially considering the aforementioned factors.

Also provided is one or more conjugates, compounds or compositions of the invention as disclosed herein for use in diagnostic medicine.

The conjugates of the invention may be useful in the treatment or diagnosis of medical indications associated with elevated FAP expression in human tissue. The conjugates of the invention may be useful in the treatment or diagnosis of cancer. The conjugates of the invention may be useful in (i) the detection of smaller primary tumors, thus allowing earlier diagnosis, (ii) the detection of smaller metastasis, thus affording a better assessment of tumor stage, (iii) providing precise intra-operative guidance facilitating complete surgical removal of tumor tissue, (iv) providing better differentiation between inflammation and tumor tissue, (v) providing more precise staging of patients with tumors, (vi) providing better follow up of tumor lesions after antitumor therapy, and (vii) as theranostic agents for diagnosis and therapy. Furthermore, the molecules can be used for the diagnosis and treatment of non-malignant diseases such as chronic inflammation, atherosclerosis, fibrosis, tissue remodeling and keloid disorders.

The conjugates of the invention may be for use in the diagnosis or treatment of a disease characterized by overexpression of fibroblast activation protein (FAP) in an animal or a human subject. The disease characterized by overexpression of fibroblast activation protein (FAP) may be selected from the group consisting of cancer, chronic inflammation, atherosclerosis, fibrosis, tissue remodelling and keloid disorder. The cancer may be selected from the group consisting of breast cancer, pancreatic cancer, small intestine cancer, colon cancer, rectal cancer, lung cancer, head and neck cancer, ovarian cancer, hepatocellular carcinoma, esophageal cancer, hypopharynx cancer, nasopharynx cancer, larynx cancer, myeloma cells, bladder cancer, cholangiocellular carcinoma, clear cell renal carcinoma, neuroendocrine tumor, oncogenic osteomalacia, sarcoma, CUP (carcinoma of unknown primary), thymus carcinoma, desmoid tumors, glioma, astrocytoma, cervix carcinoma and prostate cancer.

Preferred uses in medicine are in nuclear medicine such as nuclear diagnostic imaging, also named nuclear molecular imaging, and/or targeted radiotherapy of diseases associated with an overexpression, of FAP on the diseased tissue.

Also provided is a conjugate, compound or composition of the invention as defined herein for use in a method of diagnosing and/or staging cancer.

The term “treatment”, in relation to the uses of any of the conjugates or compounds described herein, is used to describe any form of intervention where a compound is administered to a subject suffering from, or at risk of suffering from, or potentially at risk of suffering from the disease or disorder in question. Thus, the term “treatment” covers both preventative (prophylactic) treatment and treatment where measurable or detectable symptoms of the disease or disorder are being displayed.

The term “effective therapeutic amount” (for example in relation to methods of treatment of a disease or condition) refers to an amount of the compound which is effective to produce a desired therapeutic effect.

Terms such as “alkyl”, “hydrocarbon” and “cycloalkyl” are all used in their conventional sense (e.g. as defined in the IUPAC Gold Book), unless indicated otherwise. “Optionally substituted” as applied to any group means that the said group may if desired be substituted with one or more substituents, which may be the same or different.

To the extent that any of the compounds described have chiral centers, the present invention extends to all optical isomers of such compounds, whether in the form of racemates or resolved enantiomers. The invention described herein relates to all crystal forms, solvates and hydrates of any of the disclosed compounds however so prepared. To the extent that any of the compounds disclosed herein have acid or basic centers such as carboxylates or amino groups, then all salt forms of said compounds are included herein. In the case of pharmaceutical uses, the salt should be seen as being a pharmaceutically acceptable salt.

Salts or pharmaceutically acceptable salts that may be mentioned include acid addition salts and base addition salts as well as salt forms arising due to the presence of the chelated nonradioactive or radioactive cation. Such salts may be formed by conventional means, for example by reaction of a free acid or a free base form of a compound with one or more equivalents of an appropriate acid or base, optionally in a solvent, or in a medium in which the salt is insoluble, followed by removal of said solvent, or said medium, using standard techniques (e.g. in vacuo, by freeze-drying or by filtration). Salts may also be prepared by exchanging a counter-ion of a compound in the form of a salt with another counter-ion, for example using a suitable ion exchange resin.

Further to the suitable chelated nonradioactive or radioactive cations described herein above, further examples of pharmaceutically acceptable salts include acid addition salts derived from mineral acids and organic acids, and salts derived from metals such as sodium, magnesium, potassium and calcium.

Examples of acid addition salts include acid addition salts formed with acetic, 2,2-dichloroacetic, adipic, alginic, aryl sulfonic acids (e.g. benzenesulfonic, naphthalene-2-sulfonic, naphthalene-1,5-disulfonic and p-toluenesulfonic), ascorbic (e.g. L-ascorbic), L-aspartic, benzoic, 4-acetamidobenzoic, butanoic, (+) camphoric, camphor-sulfonic, (+)-(1S)-camphor-10-sulfonic, capric, caproic, caprylic, cinnamic, citric, cyclamic, dodecylsulfuric, ethane-1,2-disulfonic, ethanesulfonic, 2-hydroxyethanesulfonic, formic, fumaric, galactaric, gentisic, glucoheptonic, gluconic (e.g. D-gluconic), glucuronic (e.g. D-glucuronic), glutamic (e g. L-glutamic), α-oxoglutaric, glycolic, hippuric, hydrobromic, hydrochloric, hydriodic, isethionic, lactic (e g. (+)-L-lactic and (±)-DL-lactic), lactobionic, maleic, malic (e.g. (−)-L-malic), malonic, (±)-DL-mandelic, metaphosphoric, methanesulfonic, 1-hydroxy-2-naphthoic, nicotinic, nitric, oleic, orotic, oxalic, palmitic, pamoic, phosphoric, propionic, L-pyroglutamic, salicylic, 4-amino-salicylic, sebacic, stearic, succinic, sulfuric, tannic, tartaric (e.g. (+)-L-tartaric), thiocyanic, undecylenic and valeric acids.

Also encompassed are any solvates of the conjugates or compounds and their salts. Preferred solvates are solvates formed by the incorporation into the solid state structure (e g. crystal structure) of the compounds of the invention of molecules of a non-toxic pharmaceutically acceptable solvent (referred to below as the solvating solvent). Examples of such solvents may include water, alcohols (such as ethanol, isopropanol and butanol) and dimethylsulfoxide. Solvates can be prepared by recrystallising the compounds of the invention with a solvent or mixture of solvents containing the solvating solvent. Whether or not a solvate has been formed in any given instance can be determined by subjecting crystals of the compound to analysis using well known and standard techniques such as thermogravimetric analysis (TGA), differential scanning calorimetry (DSC) and X-ray crystallography.

The solvates can be stoichiometric or non-stoichiometric solvates. Particular solvates may be hydrates, and examples of hydrates include hemihydrates, monohydrates and dihydrates. For a more detailed discussion of solvates and the methods used to make and characterise them, see Bryn et al, Solid-State Chemistry of Drugs, Second Edition, published by SSCI, Inc of West Lafayette, IN, USA, 1999, ISBN 0-967-06710-3.

The conjugates of the invention may contain one or more isotopic substitutions, and a reference to a particular element includes within its scope all isotopes of the element. For example, a reference to hydrogen includes within its scope ¹H, ²H (D), and ³H (T). Similarly, references to carbon and oxygen include within their scope respectively ¹²C, ¹³C and ¹⁴C and ¹⁶O and ¹⁸O. In an analogous manner, a reference to a particular functional group also includes within its scope isotopic variations, unless the context indicates otherwise. For example, a reference to an alkyl group such as an ethyl group or an alkoxy group such as a methoxy group also covers variations in which one or more of the hydrogen atoms in the group is in the form of a deuterium or tritium isotope, e.g. as in an ethyl group in which all five hydrogen atoms are in the deuterium isotopic form (a perdeuteroethyl group) or a methoxy group in which all three hydrogen atoms are in the deuterium isotopic form (a trideuteromethoxy group). The isotopes may be radioactive or non-radioactive.

### SELECTED EMBODIMENTS

Some embodiments of the invention include:

1. Ligand-SIFA conjugates comprising, within a single molecule, two separate moieties (a) and (b), wherein: (a) is one or more ligands which are capable of binding to Fibroblast Activation Protein (FAP); and, (b) is a silicon-fluoride acceptor (SIFA) moiety which comprises a covalent bond between a silicon and a fluorine atom and which is optionally labelled with ¹⁸F; and, wherein said ligand-SIFA conjugate optionally includes within said single molecule: (c) one or more chelating moieties (CM), optionally containing a chelated nonradioactive cation or radioactive cation; or, (d) one or more ligands which are capable of binding to prostate-specific membrane antigen (PSMA); or, (e) a combination of both (c) said one or more chelating moieties (CM) and (d) said one or more PSMA ligands; or a pharmaceutically or diagnostically acceptable salt or solvate thereof.

2. Ligand-SIFA conjugates according to embodiment 1 comprising, within a single molecule, two separate moieties: (a) one or more ligand(s) which is capable of binding to Fibroblast Activation Protein (FAP), and, (b) a silicon-fluoride acceptor (SIFA) moiety which comprises a covalent bond between a silicon and a fluorine atom and which is optionally labelled with ¹⁸F; or a pharmaceutically or diagnostically acceptable salt or solvate thereof.

3. Ligand-SIFA conjugates according to embodiment 1 comprising, within a single molecule, three separate moieties: (a) one or more ligands which are capable of binding to Fibroblast Activation Protein (FAP); (b) a silicon-fluoride acceptor (SIFA) moiety which comprises a covalent bond between a silicon and a fluorine atom and which is optionally labelled with ¹⁸F; and (c) one or more chelating moieties (CM), optionally containing a chelated nonradioactive or radioactive cation; or a pharmaceutically or diagnostically acceptable salt or solvate thereof.

4. Ligand-SIFA conjugates according to embodiment 1 comprising, within a single molecule three separate moieties: (a) one or more ligands which are capable of binding to Fibroblast Activation Protein (FAP); (b) a silicon-fluoride acceptor (SIFA) moiety which comprises a covalent bond between a silicon and a fluorine atom and which is optionally labelled with ¹⁸F; and (d) one or more ligands which are capable of binding to prostate-specific membrane antigen (PSMA); or a pharmaceutically or diagnostically acceptable salt or solvate thereof.

5. Ligand-SIFA conjugates according to embodiment 1 comprising, within a single molecule four separate moieties: (a) one or more ligands which are capable of binding to Fibroblast Activation Protein (FAP); (b) a silicon-fluoride acceptor (SIFA) moiety which comprises a covalent bond between a silicon and a fluorine atom and which is optionally labelled with ¹⁸F; (c) one or more chelating moieties (CM), optionally containing a chelated nonradioactive or radioactive cation; and (d) one or more ligands which are capable of binding to prostate-specific membrane antigen (PSMA); or a pharmaceutically or diagnostically acceptable salt or solvate thereof.

6. Ligand-SIFA conjugates according to embodiments 1 to 5 wherein the one or more FAP ligand(s) is as defined in any one of claims 3, and 13 to 21.

7. Ligand-SIFA conjugates according to embodiments 1 to 6 wherein the SIFA moiety is as defined in any one of claims 6, 7 and 12.

8. Ligand-SIFA conjugates according to embodiments 1, 3, 5, 6 and 7 wherein the one or more chelating moieties (CM) is as defined in any one of claims 8, 9, 10 and 11.

9. Ligand-SIFA conjugates according to embodiments 1, 4, 5, 6, 7 and 8 wherein the one or more PSMA ligand(s) is selected from a structure of formulae PSMA 1, PSMA 2, PSMA 3, PSMA 4 and PSMA 5 as identified herein.

10. A pharmaceutical or diagnostic composition comprising or consisting of one or more conjugates or compounds according to any one of embodiments 1 to 9.

11. Ligand-SIFA conjugates according to any one of embodiments 1 to 10 for use in medicine.

12. Ligand-SIFA conjugates according to any one of embodiments 1 to 10 for use as a cancer diagnostic or imaging agent.

13. A method of imaging and/or diagnosing cancer comprising administering a ligand-SIFA conjugate according to any one of embodiments 1 to 10 to a patient in need thereof.

14. Ligand-SIFA conjugates according to any one of embodiments 1 to 10 for use in the treatment of cancer.

15. Ligand-SIFA conjugates according to any one of embodiments 1 to 10 for use in the diagnosis or treatment of cancer, chronic inflammation, atherosclerosis, fibrosis, tissue remodelling and keloid disorder.

16. Ligand-SIFA conjugates according to any one of embodiments 1 to 10 for use in the diagnosis or treatment of cancer wherein the cancer is selected from the group consisting of breast cancer, pancreatic cancer, small intestine cancer, colon cancer, rectal cancer, lung cancer, head and neck cancer, ovarian cancer, hepatocellular carcinoma, esophageal cancer, hypopharynx cancer, nasopharynx cancer, larynx cancer, myeloma cells, bladder cancer, cholangiocellular carcinoma, clear cell renal carcinoma, neuroendocrine tumor, oncogenic osteomalacia, sarcoma, CUP (carcinoma of unknown primary), thymus carcinoma, desmoid tumors, glioma, astrocytoma, cervix carcinoma and prostate cancer.

Highly preferred conjugates of the invention include the conjugates of Example 1 to 13a shown in Table 1 below.

### EXAMPLES AND PREPARATIONS

The following non-restrictive examples illustrate the invention. The synthesis of the conjugates of some embodiments of the invention and of the intermediates for use therein are illustrated by the following non-limiting Examples and Preparations.

Examples of conjugates of the invention include those shown in Table 1 below. The conjugate of the invention may be selected from any one of Example 1 to 13a shown in Table 1.

TABLE 1
Example Conjugates

| | |
|---|---|
| 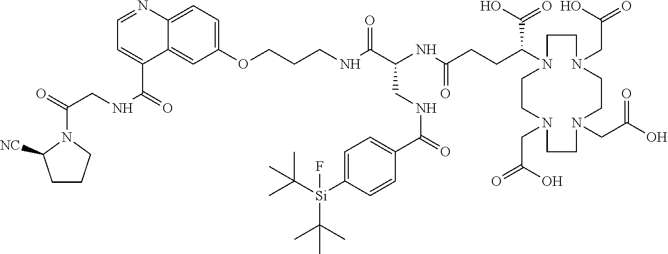 [SMILES: CC(C)(C)[Si](F)(c1ccc(C(=O)NC[C@@H](NC(=O)CC[C@H](C(=O)O)N2CCN(CC(=O)O)CCN(CC(=O)O)CCN(CC(=O)O)CC2)C(=O)NCCCOc2ccc3nccc(C(=O)NCC(=O)N4CCC[C@H]4C#N)c3c2)cc1)C(C)(C)C] | Example 1 |
| 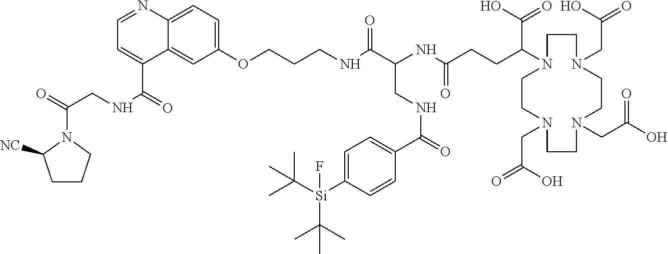 [SMILES: CC(C)(C)[Si](F)(c1ccc(C(=O)NCC(NC(=O)CCC(C(=O)O)N2CCN(CC(=O)O)CCN(CC(=O)O)CCN(CC(=O)O)CC2)C(=O)NCCCOc2ccc3nccc(C(=O)NCC(=O)N4CCC[C@H]4C#N)c3c2)cc1)C(C)(C)C] | Example 1a |
| 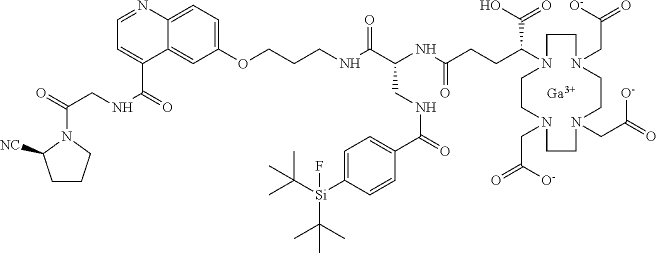 | Example 2 |
| 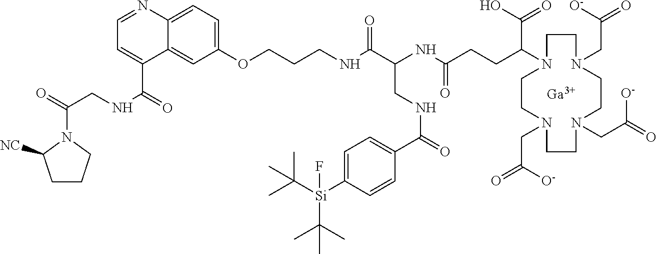 | Example 2a |
| 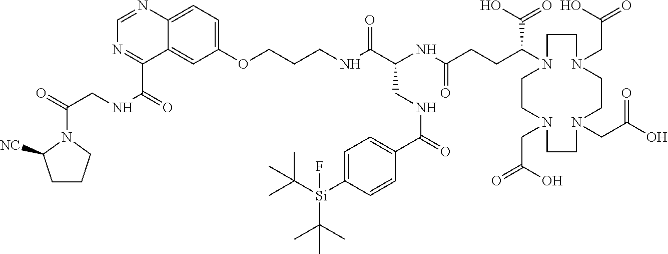 [SMILES: CC(C)(C)[Si](F)(c1ccc(C(=O)NC[C@@H](NC(=O)CC[C@H](C(=O)O)N2CCN(CC(=O)O)CCN(CC(=O)O)CCN(CC(=O)O)CC2)C(=O)NCCCOc2ccc3ncnc(C(=O)NCC(=O)N4CCC[C@H]4C#N)c3c2)cc1)C(C)(C)C] | Example 3 |
| 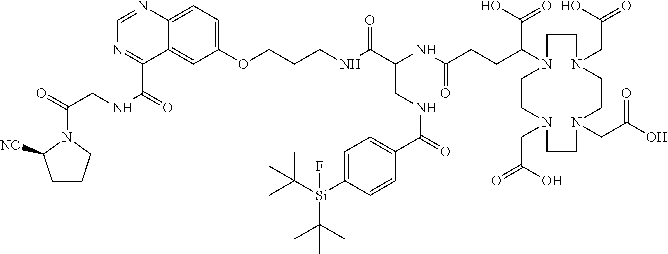 [SMILES: CC(C)(C)[Si](F)(c1ccc(C(=O)NCC(NC(=O)CCC(C(=O)O)N2CCN(CC(=O)O)CCN(CC(=O)O)CCN(CC(=O)O)CC2)C(=O)NCCCOc2ccc3ncnc(C(=O)NCC(=O)N4CCC[C@H]4C#N)c3c2)cc1)C(C)(C)C] | Example 3a |
| 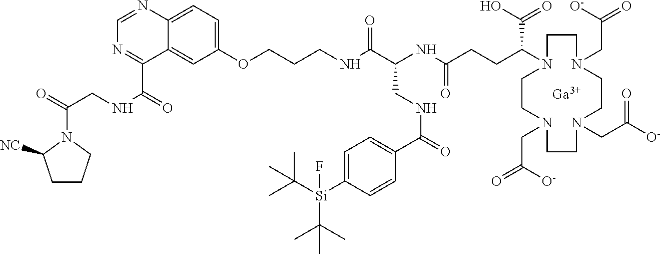 | Example 4 |
| 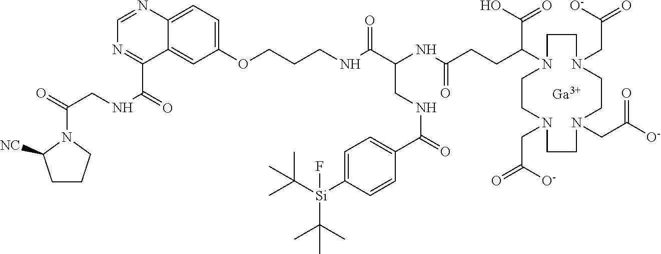 | Example 4a |
| 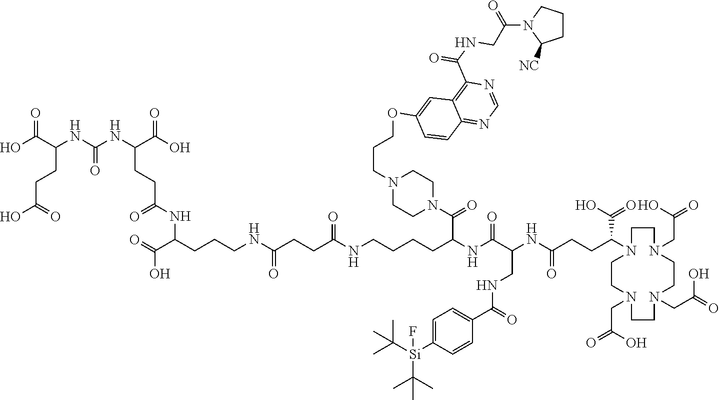 [SMILES: CC(C)(C)[Si](F)(c1ccc(C(=O)NCC(NC(=O)CC[C@H](C(=O)O)N2CCN(CC(=O)O)CCN(CC(=O)O)CCN(CC(=O)O)CC2)C(=O)NC(CCCCNC(=O)CCC(=O)NCCCC(NC(=O)CCC(NC(=O)NC(CCC(=O)O)C(=O)O)C(=O)O)C(=O)O)C(=O)N2CCN(CCCOc3ccc4ncnc(C(=O)NCC(=O)N5CCC[C@H]5C#N)c4c3)CC2)cc1)C(C)(C)C] | Example 5 |
| 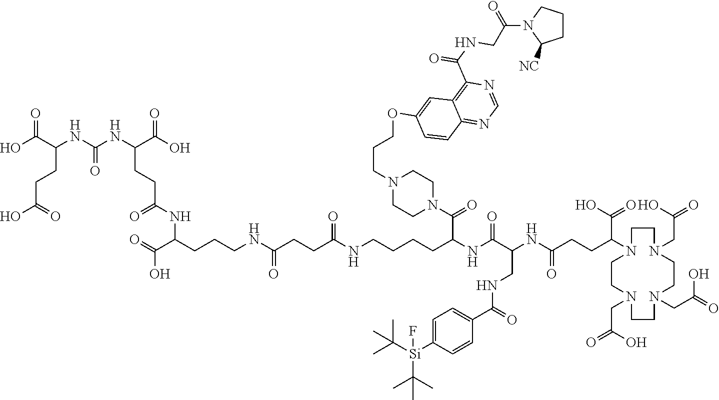 [SMILES: CC(C)(C)[Si](F)(c1ccc(C(=O)NCC(NC(=O)CCC(C(=O)O)N2CCN(CC(=O)O)CCN(CC(=O)O)CCN(CC(=O)O)CC2)C(=O)NC(CCCCNC(=O)CCC(=O)NCCCC(NC(=O)CCC(NC(=O)NC(CCC(=O)O)C(=O)O)C(=O)O)C(=O)O)C(=O)N2CCN(CCCOc3ccc4ncnc(C(=O)NCC(=O)N5CCC[C@H]5C#N)c4c3)CC2)cc1)C(C)(C)C] | Example 5a |
| 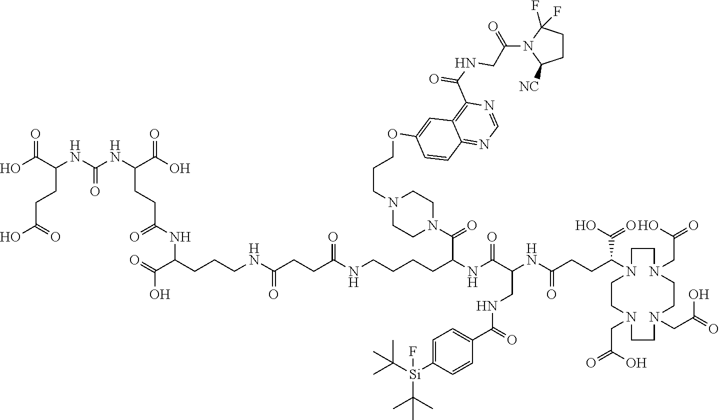 [SMILES: CC(C)(C)[Si](F)(c1ccc(C(=O)NCC(NC(=O)CC[C@H](C(=O)O)N2CCN(CC(=O)O)CCN(CC(=O)O)CCN(CC(=O)O)CC2)C(=O)NC(CCCCNC(=O)CCC(=O)NCCCC(NC(=O)CCC(NC(=O)NC(CCC(=O)O)C(=O)O)C(=O)O)C(=O)O)C(=O)N2CCN(CCCOc3ccc4ncnc(C(=O)NCC(=O)N5[C@H](C#N)CCC5(F)F)c4c3)CC2)cc1)C(C)(C)C] | Example 6 |
| 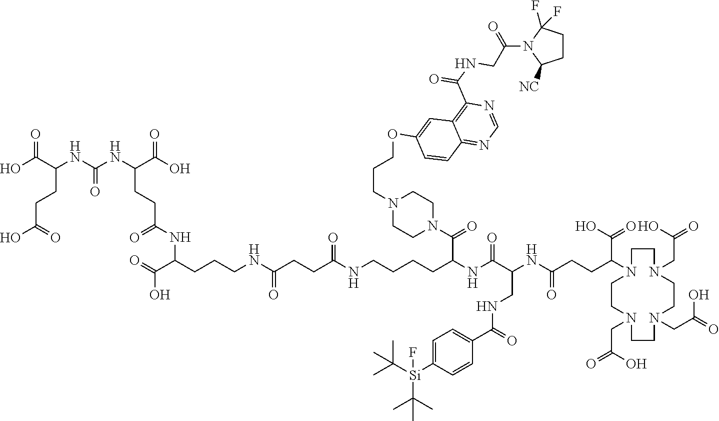 [SMILES: CC(C)(C)[Si](F)(c1ccc(C(=O)NCC(NC(=O)CCC(C(=O)O)N2CCN(CC(=O)O)CCN(CC(=O)O)CCN(CC(=O)O)CC2)C(=O)NC(CCCCNC(=O)CCC(=O)NCCCC(NC(=O)CCC(NC(=O)NC(CCC(=O)O)C(=O)O)C(=O)O)C(=O)O)C(=O)N2CCN(CCCOc3ccc4ncnc(C(=O)NCC(=O)N5[C@H](C#N)CCC5(F)F)c4c3)CC2)cc1)C(C)(C)C] | Example 6a |
| 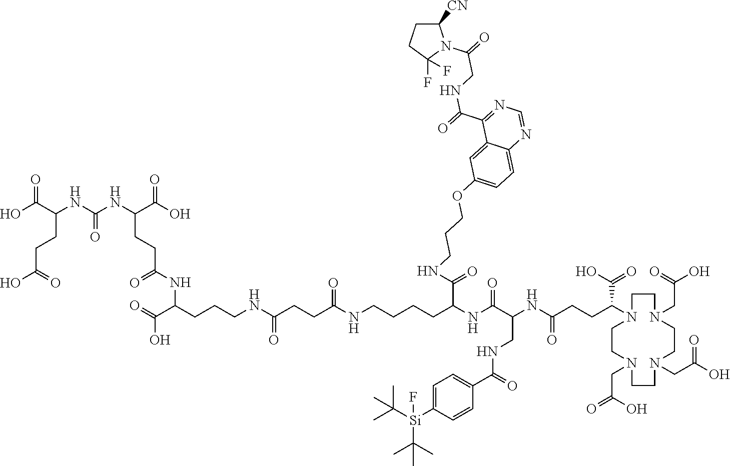 [SMILES: CC(C)(C)[Si](F)(c1ccc(C(=O)NCC(NC(=O)CC[C@H](C(=O)O)N2CCN(CC(=O)O)CCN(CC(=O)O)CCN(CC(=O)O)CC2)C(=O)NC(CCCCNC(=O)CCC(=O)NCCCC(NC(=O)CCC(NC(=O)NC(CCC(=O)O)C(=O)O)C(=O)O)C(=O)O)C(=O)NCCCOc2ccc3ncnc(C(=O)NCC(=O)N4[C@H](C#N)CCC4(F)F)c3c2)cc1)C(C)(C)C] | Example 7 |
| 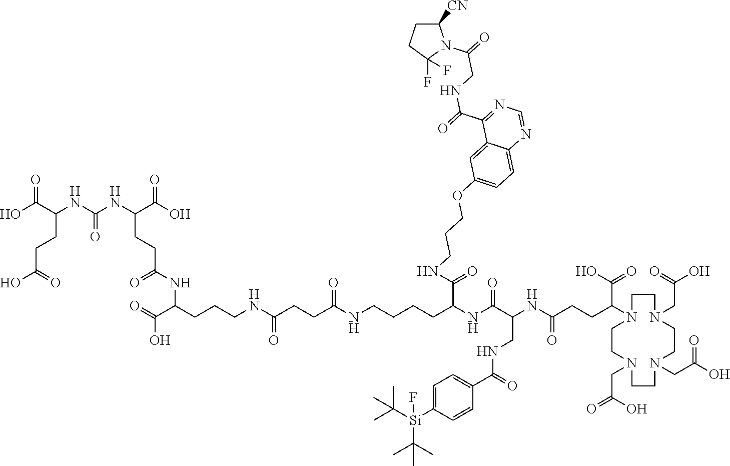 [SMILES: CC(C)(C)[Si](F)(c1ccc(C(=O)NCC(NC(=O)CCC(C(=O)O)N2CCN(CC(=O)O)CCN(CC(=O)O)CCN(CC(=O)O)CC2)C(=O)NC(CCCCNC(=O)CCC(=O)NCCCC(NC(=O)CCC(NC(=O)NC(CCC(=O)O)C(=O)O)C(=O)O)C(=O)O)C(=O)NCCCOc2ccc3ncnc(C(=O)NCC(=O)N4[C@H](C#N)CCC4(F)F)c3c2)cc1)C(C)(C)C] | Example 7a |
| 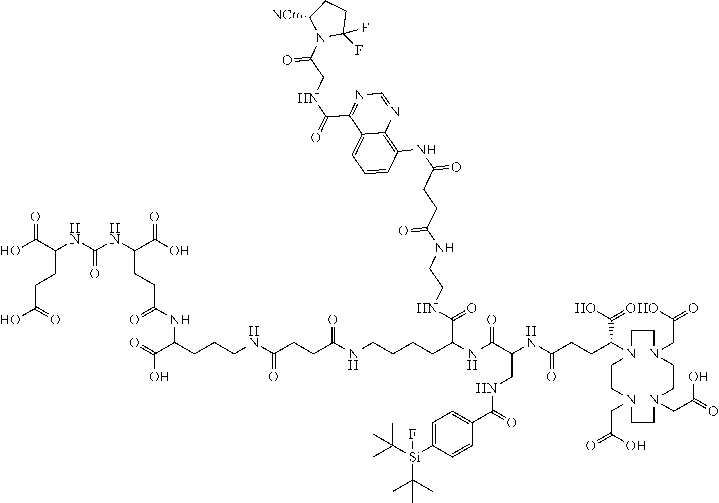 [SMILES: CC(C)(C)[Si](F)(c1ccc(C(=O)NCC(NC(=O)CC[C@H](C(=O)O)N2CCN(CC(=O)O)CCN(CC(=O)O)CCN(CC(=O)O)CC2)C(=O)NC(CCCCNC(=O)CCC(=O)NCCCC(NC(=O)CCC(NC(=O)NC(CCC(=O)O)C(=O)O)C(=O)O)C(=O)O)C(=O)NCCNC(=O)CCC(=O)Nc2cccc3c(C(=O)NCC(=O)N4[C@H](C#N)CCC4(F)F)ncnc23)cc1)C(C)(C)C] | Example 8 |
| 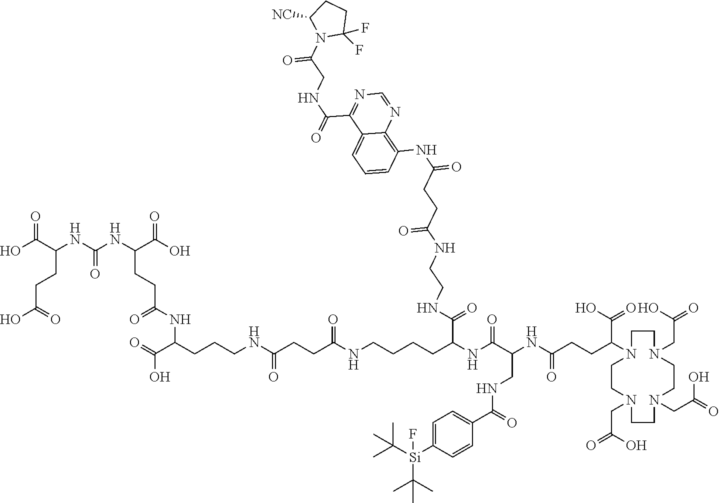 [SMILES: CC(C)(C)[Si](F)(c1ccc(C(=O)NCC(NC(=O)CCC(C(=O)O)N2CCN(CC(=O)O)CCN(CC(=O)O)CCN(CC(=O)O)CC2)C(=O)NC(CCCCNC(=O)CCC(=O)NCCCC(NC(=O)CCC(NC(=O)NC(CCC(=O)O)C(=O)O)C(=O)O)C(=O)O)C(=O)NCCNC(=O)CCC(=O)Nc2cccc3c(C(=O)NCC(=O)N4[C@H](C#N)CCC4(F)F)ncnc23)cc1)C(C)(C)C] | Example 8i |
| 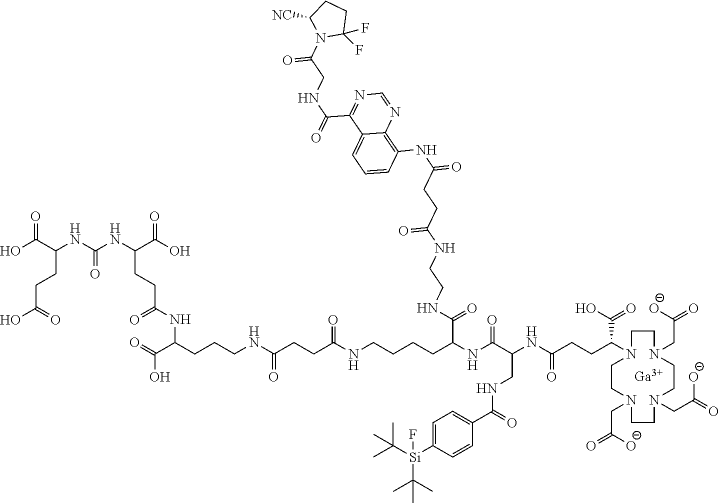 | Example 8a |
| 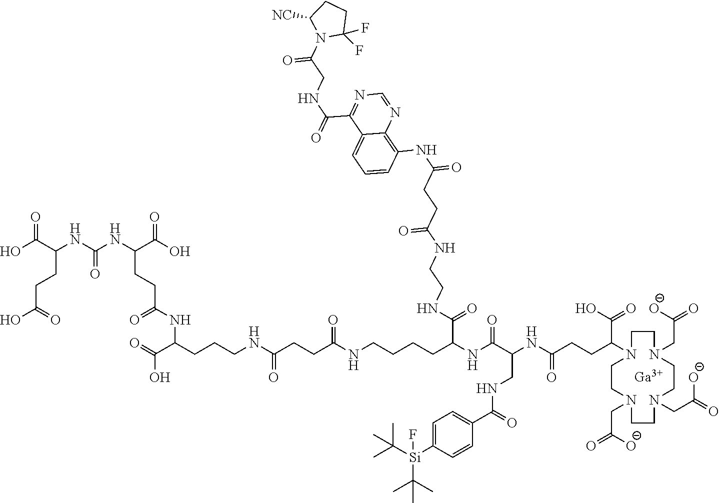 | Example 8ai |
| 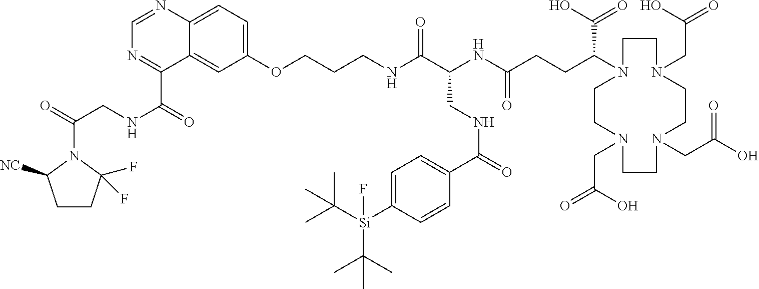 [SMILES: CC(C)(C)[Si](F)(c1ccc(C(=O)NC[C@@H](NC(=O)CC[C@H](C(=O)O)N2CCN(CC(=O)O)CCN(CC(=O)O)CCN(CC(=O)O)CC2)C(=O)NCCCOc2ccc3ncnc(C(=O)NCC(=O)N4[C@H](C#N)CCC4(F)F)c3c2)cc1)C(C)(C)C] | Example 9 |
| 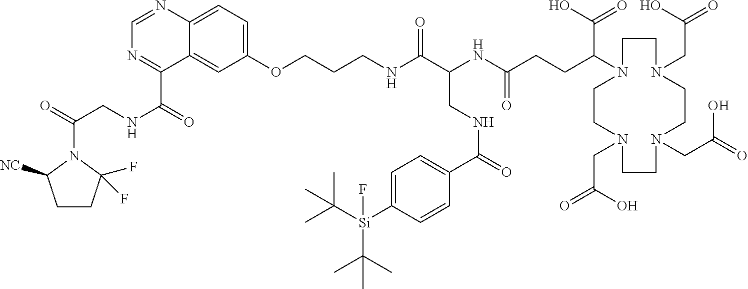 [SMILES: CC(C)(C)[Si](F)(c1ccc(C(=O)NCC(NC(=O)CCC(C(=O)O)N2CCN(CC(=O)O)CCN(CC(=O)O)CCN(CC(=O)O)CC2)C(=O)NCCCOc2ccc3ncnc(C(=O)NCC(=O)N4[C@H](C#N)CCC4(F)F)c3c2)cc1)C(C)(C)C] | Example 9a |
| 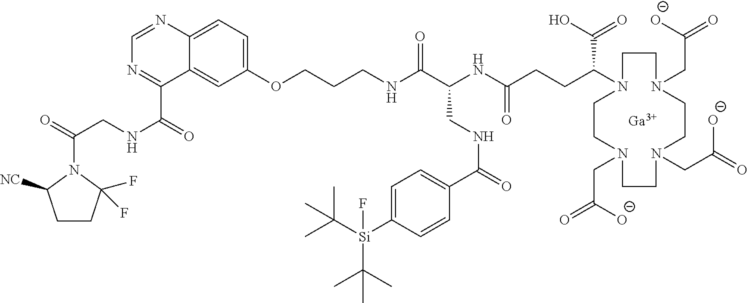 | Example 10 |
| 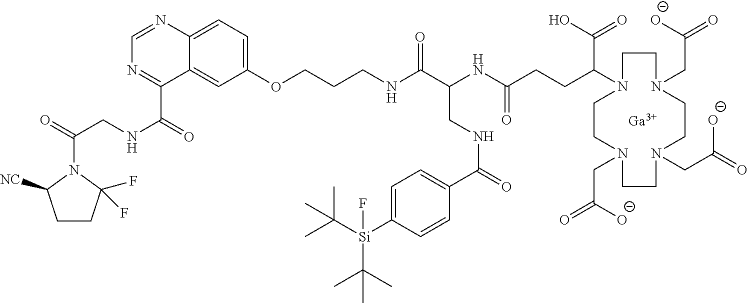 | Example 10a |
| 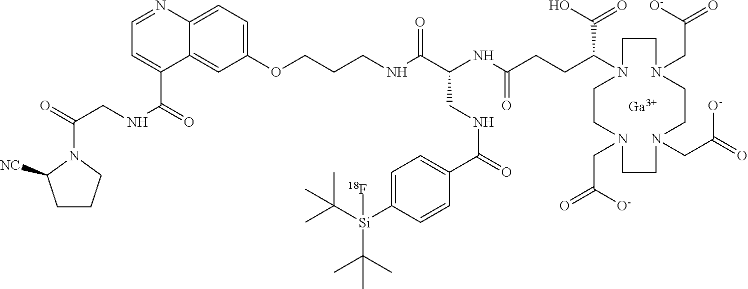 | Example 11 |
| 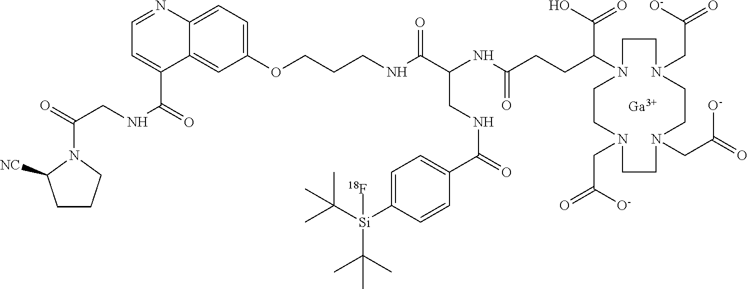 | Example 11a |
| 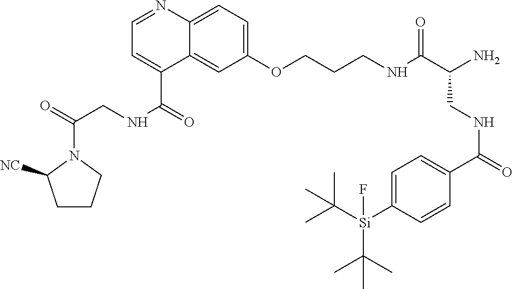 [SMILES: CC(C)(C)[Si](F)(c1ccc(C(=O)NC[C@@H](N)C(=O)NCCCOc2ccc3nccc(C(=O)NCC(=O)N4CCC[C@H]4C#N)c3c2)cc1)C(C)(C)C] | Example 12 |
| 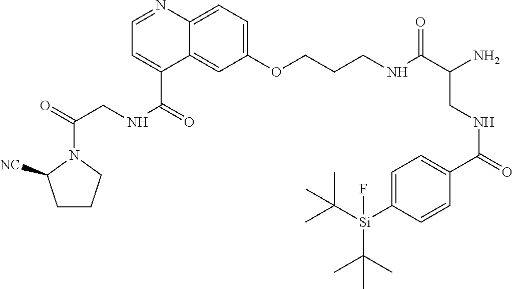 [SMILES: CC(C)(C)[Si](F)(c1ccc(C(=O)NCC(N)C(=O)NCCCOc2ccc3nccc(C(=O)NCC(=O)N4CCC[C@H]4C#N)c3c2)cc1)C(C)(C)C] | Example 12a |
| 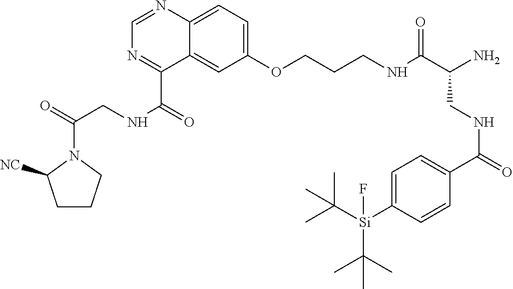 [SMILES: CC(C)(C)[Si](F)(c1ccc(C(=O)NC[C@@H](N)C(=O)NCCCOc2ccc3ncnc(C(=O)NCC(=O)N4CCC[C@H]4C#N)c3c2)cc1)C(C)(C)C] | Example 13 |
| 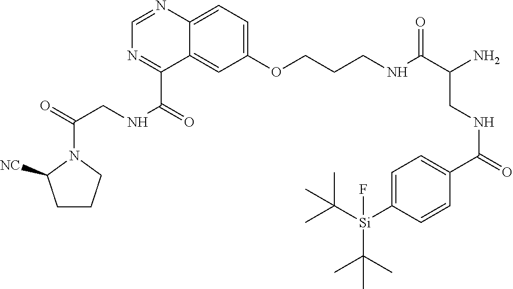 [SMILES: CC(C)(C)[Si](F)(c1ccc(C(=O)NCC(N)C(=O)NCCCOc2ccc3ncnc(C(=O)NCC(=O)N4CCC[C@H]4C#N)c3c2)cc1)C(C)(C)C] | Example 13a |

### General Method

Certain conjugates of the invention may be prepared in accordance with the general scheme below, where FAP comprises an FAP binding moiety, PSMA comprises a PSMA binding moiety, L represents a linker moiety, SIFA represents a SIFA-containing moiety, CM comprises a chelating moiety and CM(M) comprises a chelating moiety with a chelated metal cation.

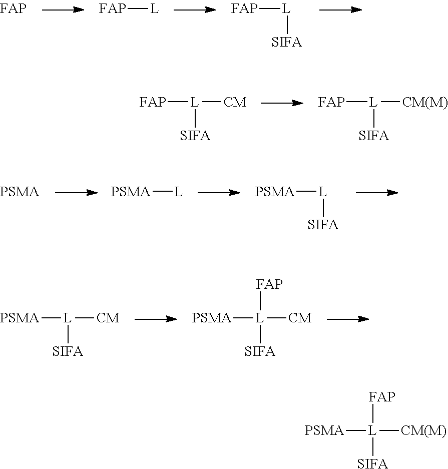

### Materials and Methods

Where no preparative routes are included, the relevant intermediate is commercially available Commercial grade reagents were utilized without further purification, unless otherwise stated. The purity of compounds was determined by HPLC, and all final target compounds had purities of >95%, unless otherwise stated. Proton nuclear magnetic resonance (¹H NMR) spectra were recorded in the deuterated solvents specified on a Varian 400 spectrometer operating at 400 MHz. Mass spectra were determined by using Shimadzu LCMS 2020 with N-Series DUIS (ESI) system using positive-negative switching. HPLC spectra were determined by using Agilent 1200 series.

Room temperature includes 20 to 25° C.

### Synthesis of the Silicon-Fluoride Acceptor Reagent (SiFA-BA)

The silicon-fluoride acceptor reagent used herein was 4-(di-tert-butylfluorosilyl)benzoic acid (SiFA-BA):

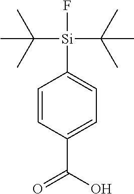 [SMILES: CC(C)(C)[Si](F)(c1ccc(C(=O)O)cc1)C(C)(C)C]

SiFA-BA was synthesized according to a previously published procedure (L. Iovkova et al. Chem. Eur. J. 2009, 15, 2140-2147) as depicted in the below scheme. All reactions were carried out in dried reaction vessels under argon using a vacuum gas manifold.

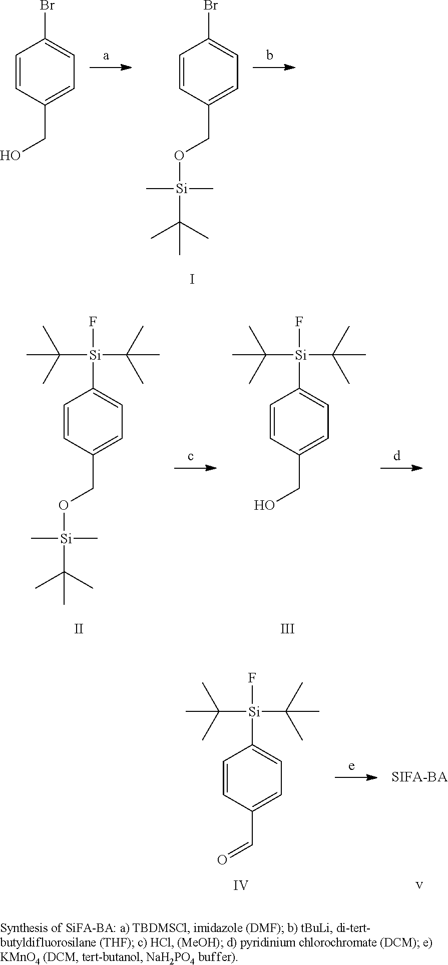

### Synthesis of Conjugates of Example 1 and Example 2

There is shown in the scheme below an exemplary synthetic procedure to obtain the conjugates of the invention of Example 1 and Example 2, as detailed in Table 1.

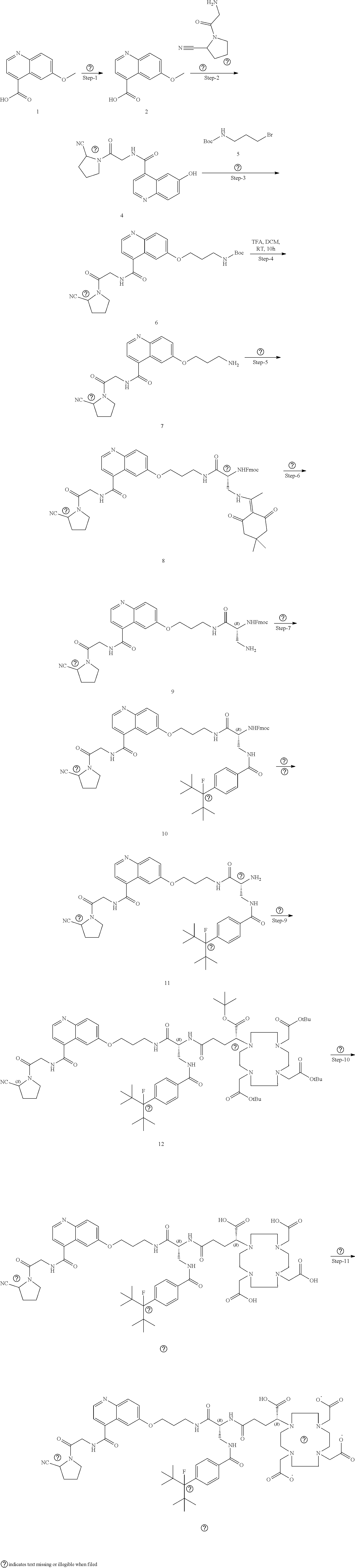

### Synthesis of Intermediates

### Preparation 1

### 6-Hydroxyquinoline-4-carboxylic acid (2)

6-methoxyquinoline-4-carboxylic acid (5 g, 0.0246 mol; BLD-pharma) and solution of HBr (48% in water, 75 ml) in a seal tube was heated to 140° C. for 4 h with vigorous stirring. The progress of the reaction was monitored by thin-layer chromatography (TLC) analysis. After completion, the reaction mixture was cooled to room temperature and concentrated under reduced pressure. The residue was dissolved in water (20 ml) and resulting solution was basified (pH=7.5) using 4 N aqueous sodium hydroxide (NaOH) solution. The precipitate formed was removed by filtration and the filtrate was concentrated to dryness under reduced pressure. The residue was triturated with methanol (20 ml×3 and diethyl ether (20 ml). The resulting slurry was dissolved in acetonitrile (20 ml) and water (60 ml). The resulting mixture was lyophilized to obtain the title compound as a light brown solid (yield: 4.0 g, 86%). The title compound was characterized by LC-MS and ¹H NMR analysis. LC-MS, C₁₀H₇NO₃ calculated 189.04; observed 190.25 [M+H]⁺. ¹H NMR (400 MHz, DMSO): δ 8.46 (d, J=4.0 Hz, 1H), 8.10 (s, 1H), 7.70 (d, J=9.2 Hz, 1H), 7.32 (d, J=4.0 Hz, 1H), 7.17-7.14 (m, 1H).

### Preparation 2

### (S)—N-(2-(2-Cyanopyrrolidin-1-yl)-2-oxoethyl-6-hydroxyquinoline-4-carboxamide (4)

N, N-Diisopropylethylamine (3.5 ml, 0.0198 mol), 1-hydroxy-7-azabenzotriazole (4.0 g, 0.264 mol; Spectrochem) and 2-(1H-Benzotriazole-1-yl)-1,1,3,3-tetramethyluronium (6.0 g, 0.0158 mol; Spectrochem) were added to a stirred solution of the title compound of Preparation 1 (2.5 g, 0.0132 mol) and (S)-1-glycylpyrrolidine-2-carbonitrile (3.0 g, 0.0198 mol, BLD-pharma) in dry N,N-dimethyl formamide (DMF, 50 ml) under nitrogen. The resulting mixture was stirred at room temperature for 16 h. The progress of the reaction was monitored by thin-layer chromatography (TLC) analysis. After completion of the reaction, water (30 ml) was added, and the resulting mixture was extracted with ethyl acetate (50 ml×2). The combined organic layer was dried over anhydrous sodium sulphate and concentrated under reduced pressure. The crude compound was purified by silica-gel (230-400 silica) column chromatography using 5% methanol in dichloromethane to afford the title compound as a yellow solid (yield: 1.9 g, 45%). The title compound was characterized by LC-MS analysis. LC-MS, C₁₇H₁₆N₄O₃ calculated 324.12; observed 325.30 [M+H]⁺.

### Preparation 3

### tert-Butyl-(S)-3-((4-((2-(2-cyanopyrrolidin-1-yl)-2-oxoethyl)carbamoyl)quinolin-6-yl)oxy)propyl)carbamate (6)

tert-Butyl(3-bromopropyl)carbamate (0.87 g, 0.0037 mol) was added portion wise to a solution of the title compound of Preparation 2 (1 g, 0.0031 mol) and potassium carbonate (0.51 g, 0.0037 mol) in N,N-dimethyl formamide (DMF, 10 ml) under nitrogen. The resulting mixture was stirred at 60° C. for 16 h. The progress of the reaction was monitored by TLC analysis. After completion, the reaction mixture was diluted with water (50 ml) and extracted with ethyl acetate (30 ml×3). The combined organic layer was washed with brine and dried over anhydrous sodium sulphate. The solution was filtered, and the filtrate concentrated under reduced pressure to afford a light brown crude compound. The crude compound was purified by silica gel (230-400 mesh) eluting with 0-5% methanol in dichloromethane to afford the title compound as a yellow solid (yield: 0.75 g, 50%). The title compound was characterized by LC-MS and ¹H NMR analysis. LC-MS, C₂₅H₃₁N₅O₅ calculated 481.23; observed 482.40 [M+H]⁺. ¹H NMR (400 MHz, CDCl₃): δ 8.79 (d, J=4.4 Hz, 1H), 7.99 (d, J=9.2 Hz, 1H), 7.62 (bs, 1H), 7.51 (d, J=4.0 Hz, 1H), 7.38-7.35 (m, 1H), 7.30 (bs, 1H), 5.17 (bs, 1H), 4.80 (bs, 1H), 4.46-4.40 (m, 1H), 4.31-4.27 (m, 1H), 4.17 (m, 2H), 3.73 (bs, 1H), 3.58-3.54 (m, 1H), 3.38-3.37 (m, 2H), 2.37-2.26 (m, 4H), 2.05 (bs, 2H), 1.44 (s, 9H).

### Preparation 4

### (S)-6-(3-Aminopropoxy)-N-(2-(2-cyanopyrrolidin-1-yl)-2-oxoethyl)quinoline-4-carboxamide (7)

Trifluoroacetic acid (0.25 ml, 0.0032 mol) was added to a solution of the title compound of Preparation 3 (0.75 g, 0.0016 mol) in dichloromethane (7.5 ml) under nitrogen. The resulting mixture was stirred at room temperature for 16 h. The progress of the reaction was monitored by thin-layer chromatography (TLC) analysis. The reaction mixture was then concentrated under reduced pressure and the residue was co-distilled (3 times) with fresh dichloromethane. Finally, the resulting residue was triturated firstly with diethyl ether (10 ml) and then with n-pentane (10 ml), and then dried under vacuo to afford the title compound as a light brown solid in quantitative yield. The crude title compound was used without further purification. The title compound was characterized by LC-MS and ¹H NMR analysis. LC-MS, C₂₀H₂₃N₅O₃ calculated 381.18; observed 382.35 [M+H]⁺. ¹H NMR (400 MHz, DMSO): δ 9.09 (d, J=5.2 Hz, 1H), 8.84 (d, J=4.0 Hz, 1H), 8.02 (d, J=9.2 Hz, 1H), 7.96-7.87 (m, 4H), 7.56-7.49 (m, 2H), 4.82 (d, J=4.0 Hz, 1H), 4.23 (s, 4H), 3.74 (bs, 1H), 3.59-3.53 (m, 1H), 3.04-3.03 (m, 2H), 2.23-2.09 (m, 5H).

### Preparation 5

(9H-Fluoren-9-yl)methyl((R)-1-((3-((4-((2-((S)-2-cyanopyrrolidin-1-yl)-2-oxoethyl)carbamoyl)quinolin-6-yl)oxy)propyl)amino)-3-((1-(4,4-dimethyl-2,6-dioxocyclohexylidene)ethyl)amino)-1-oxopropan-2-yl)carbamate (8)

2,4,6-Collidine (1.65 mL, 12.30 mmol), 1-hydroxy-7-azabenzotriazole (0.51 g, 3.674 mmol) and 2-(1H-benzotriazole-1-yl)-1,1,3,3-tetramethylaminium tetrafluoroborate (1.18 g, 3.674 mmol) were added to a stirred solution of the title compound of Preparation 4 (0.7 g, 1.84 mmol) and N-alpha-(9-fluorenylmethyloxycarbonyl)-N-beta-[(4,4-dimethyl-2,6-dioxocyclohex-1-ylidene)ethyl]-D-2,3-diaminopropionic acid (0.99 g, 2.02 mmol, ACT-China) in dry N,N-dimethyl formamide (DMF, 14 ml) under nitrogen. The resulting mixture was stirred at room temperature for 16 h. The progress of the reaction was monitored by TLC analysis. After completion of the reaction, water (30 ml) was added, and the resulting mixture was extracted with ethyl acetate (50 ml×2). The combined organic layer was dried over anhydrous sodium sulphate and concentrated under reduced pressure. The crude title compound was purified by silica-gel (230-400 silica) column chromatography using 5% methanol in dichloromethane to afford the title compound as an off-white solid (yield: 0.90 g, 51%). The title compound was characterized by LC-MS analysis. LC-MS, C₄₈H₅₁N₇O₈ calculated 853.38; observed 854.50 [M+H]⁺.

Preparation 6

(9H-Fluoren-9-yl)methyl-((R)-3-amino-1-((3-((4-((2-((S)-2-cyanopyrrolidin-1-yl)-2-oxoethyl)carbamoyl)quinolin-6-yl)oxy)propyl)amino)-1-oxopropan-2-yl)carbamate (9)

Hydrazine hydrate (25%, 1.3 ml) was added to a stirred solution of the title compound of Preparation 5 (2.6 g, 0.0027 mol) in ethanol (25 ml) and the resulting mixture was stirred at room temperature for 2 h. The progress of the reaction was monitored by thin-layer chromatography (TLC) analysis. After completion of the reaction, water (20 ml) was added, and the resulting mixture was extracted with dichloromethane (100 ml×3). The combined organic layers were dried over anhydrous sodium sulphate and concentrated under reduced pressure. The crude title compound was purified by silica-gel (230-400 silica) column chromatography using 10% methanol in dichloromethane to afford the title compound as an off-white solid (yield: 1.50 g, 78%). The title compound was characterized by LC-MS and ¹H NMR analysis. LC-MS, C₃₈H₃₉N₇O₆ calculated 689.30; observed 690.45 [M+H]⁺. ¹H NMR (400 MHz, DMSO): δ 9.03 (t, J=5.6 Hz, 1H), 8.81 (d, J=4.4 Hz, 1H), 8.12 (bs, 1H), 7.97 (d, J=8.8 Hz, 1H), 7.89-7.88 (m, 3H), 7.68 (d, J=7.6 Hz, 2H), 7.51 (d, J=4.4 Hz, 1H), 7.46-7.39 (m, 3H), 7.32 (t, J=7.2 Hz, 3H), 4.83-4.81 (m, 1H), 4.30-4.17 (m, 8H), 3.72 (bs, 4H), 3.57-3.51 (m, 1H), 3.06-2.99 (m, 2H), 2.22-2.16 (m, 2H), 2.09-2.05 (m, 2H), 1.97-1.94 (m, 3H).

Preparation 7

(9H-Fluoren-9-yl)methyl-((R)-1-((3-((4-((2-((S)-2-cyanopyrrolidin-1-yl)-2-oxoethyl)carbamoyl)quinolin-6-yl)oxy)propyl)amino)-3-(4-(di-tert-butylfluorosilyl)benzamido)-1-oxopropan-2-yl)carbamate (10)

N,N-Diisopropylethylamine (1.8 ml, 9.79 mmol), 1-hydroxy-7-azabenzotriazole (0.44 g, 3.27 mmol) and 2-(1H-benzotriazole-1-yl)-1,1,3,3-tetramethylaminium tetrafluoroborate (1.0 g, 3.27 mmol) were added to a stirred solution of the title compound of Preparation 6 (1.5 g, 2.18 mmol) and 4-(di-tert-butylfluorosilyl)benzoic acid (0.92 g, 3.27 mmol) in dry N,N-dimethyl formamide (DMF, 15 ml) under nitrogen. The resulting mixture was stirred at room temperature for 24 h and the progress of reaction monitored by thin-layer chromatography (TLC analysis. After completion of the reaction, water (100 ml) was added, and the resulting mixture was extracted with ethyl acetate (300 ml×2). The combined organic layers were dried over anhydrous sodium sulphate and concentrated under reduced pressure. The crude title compound was purified by silica-gel (230-400 silica) column chromatography using 1-10% methanol in dichloromethane to afford the title compound as an off-white solid (yield: 1.2 g, 60%). The title compound was characterized by LC-MS analysis. LC-MS, C₅₃H₆₀FN₇O₇S calculated 953.43; observed 954.65 [M+H]⁺.

Preparation 8

6-(3-((R)-2-Amino-3-(4-(di-ted-butylfluorosilyl)benzamido)propanamido)propoxy)-N-(2-((S)-2-cyanopyrrolidin-1-yl)-2-oxoethyl)quinoline-4-carboxamide (11) (Example 12)

A solution of piperidine (0.15 ml, 1.51 mmol) in N,N-dimethyl formamide (DMF, 1 ml) was added dropwise to a stirred solution of the title compound of Preparation 7 (1.2 g, 1.26 mmol) in N,N-dimethyl formamide (DMF, 12 ml). The resulting mixture was stirred at room temperature for 1 h and the reaction monitored by TLC analysis. After completion of the reaction, water (100 ml) was added, and the resulting mixture was extracted with ethyl acetate (70 ml×3). The combined organic layers were dried over anhydrous sodium sulphate and concentrated under reduced pressure. The crude title compound was purified by silica-gel (230-400 silica) column chromatography using 5-12% methanol in dichloromethane to afford the title compound as an off-white solid (yield: 0.90 g, 98%). The title compound was characterized by LC-MS and ¹H NMR analysis. LC-MS, C₃₈H₅₀FN₇O₅Si calculated 731.36; observed: 732.45 [M+H]⁺. ¹H NMR (400 MHz, DMSO): δ 9.06 (t, J=6.0 Hz, 1H), 8.81-8.77 (m, 2H), 8.26 (t, J=4.8 Hz, 1H), 8.03-7.95 (m, 3H), 7.87 (bs, 1H), 7.67 (d, J=7.6 Hz, 2H), 7.51 (d, J=4.4 Hz, 1H), 7.45 (d, J=8.8 Hz, 1H), 6.01 (bs, 2H), 4.84 (bs, 1H), 4.60 (bs, 1H), 4.25-4.16 (m, 4H), 3.74 (bs, 1H), 3.57-3.37 (m, 1H), 3.17-3.14 (m, 2H), 3.13-3.08 (m, 2H), 3.06-3.02 (m, 1H), 2.50 (s, 8H), 2.30-1.94 (m, 6H), 1.30-1.14 (m, 1H), 1.08 (s, 18H).

Preparation 9

tri-tert-Butyl 2,2′,2″-(10-((R)-1-(tert-butoxy)-5-(((R)-1-((3-((4-((2-((S)-2-cyanopyrrolidin-1-yl)-2-oxoethyl)carbamoyl)quinolin-6-yl)oxy)propyl)amino)-3-(4-(di-tert-butylfluorosilyl)benzamido)-1-oxopropan-2-yl)amino-1,5-dioxopentan-2-yl)-1,4,7,10-tetraazacyclododecane-1,4,7-triyl)triacetate (12)

N,N-Diisopropylethylamine (0.70 ml, 3.69 mmol) was added to a stirred solution of the title compound of Preparation 8 (0.90 g, 1.23 mmol) and 5-(tert-butoxy)-5-oxo-4-(4,7,10-tris(2-(tert-butoxy)-2-oxoethyl)-1,4,7,10-tetraazacyclododecan-1-yl) pentanoic acid (0.86 g, 1.23 mmol, Argonix Reagents & Intermediates) in dichloromethane (18 ml) under nitrogen. This was followed by an addition of 1-propanephosphonic anhydride solution (50% in ethyl acetate, 1.2 ml, 3.69 mmol). The resulting mixture was stirred at room temperature for 3 h and the reaction monitored by TLC analysis. After completion of the reaction, water (50 ml) was added, and the resulting mixture was extracted with dichloromethane (100 mL×3). The combined organic layers were dried over anhydrous sodium sulphate and concentrated under reduced pressure. The crude title compound was purified by silica-gel (230-400 silica) column chromatography using 5-14% methanol in dichloromethane to afford the title compound as an off-white solid (yield: 1.0 g, 57%). The product (12) was taken forward to the next step without further characterisation.

Synthesis of Conjugates of Examples 1 and 2

Preparation 10—Compound of Example 1

2,2′,2″-(10-((R)-1-Carboxy-4-(((R)-1-((3-((4-((2-((S)-2-cyanopyrrolidin-1-yl)-2-oxoethyl)carbamoyl)quinolin-6-yl)oxy)propyl)amino)-3-(4-(di-tert-butylfluorosilyl) benzamido)-1-oxopropan-2-yl)amino)-4-oxobutyl)-1,4,7,10-tetraazacyclododecane-1,4,7-triyl) triacetic acid (13)

A solution of the title compound of Preparation 9 (1.0 g, 0.71 mmol) in trifluoroacetic acid: triisopropyl silane:water (95:2.5:2.5, 15 ml) was stirred at room temperature for 16 h under nitrogen. The progress of the reaction was monitored by LC-MS analysis. After completion of the reaction, the reaction mixture was concentrated under reduced pressure to obtain a sticky liquid residue. The residue was triturated with methyl tert-butyl ether (10×3 ml times) followed with n-pentane (10 ml) to produce a solid. The solid was dried under vacuum to afford crude title compound as an off-white solid (yield: 0.83 g, 99%). The crude title compound (70 mg) was purified by reverse phase high performance liquid chromatography (HPLC) (Mobile phase A: 0.1% Formic acid in water; and Mobile phase B: acetonitrile—column: Inertsil ODS3V 250×20 mm, 5.0 μm) to produce 20 mg of the title compound as a pale-yellow solid. LC-MS, C₅₇H₈₀FN₁₁O₁₄Si calculated 1189.56; observed 1188.35 [M−H]⁻. ¹H NMR (400 MHz, DMSO): δ 11.80 (bs, 4H), 9.03 (bs, 1H), 8.80 (s, 1H), 8.47-8.33 (m, 2H), 8.14 (bs, 1H), 7.96-7.85 (m, 4H), 7.64-7.62 (m, 2H), 7.51-7.44 (m, 1H), 4.83 (bs, 1H), 4.44 (bs, 2H), 4.21-4.16 (m, 4H), 3.72 (bs, 1H), 3.53-3.40 (m, 10H), 3.10-2.80 (m, 13H), 2.33-1.71 (m, 14H), 1.02 (s, 18H). High performance liquid chromatography (HPLC): 10.361 min, 98.46%, (Column: Inertsil ODS 3V-C18 4.6×250 mm, 5 μm, Mobile phase A: 0.1% Formic acid in water; Mobile phase B: Acetonitrile).

Preparation 11—Compound of Example 2

Gallium(I) 2-(16-((R)-1-carboxy-4-(((R)-1-((3-((4-((2-((S)-2-cyanopyrrolidin-1-yl)-2-oxoethyl)carbamoyl)quinolin-6-yl)oxy)propyl)amino)-3-(4-(di-tert-butylfluorosilyl)benzamido)-1-oxopropan-2-yl)amino)-4-oxobutyl)-3,6-dioxo-4,5-dioxa-1,8,11,16-tetraazabicyclo[6,5,5]octadecan-11-yl)acetate (14, Ga—(S,R,R)—SiFA-FAP-1)

Gallium (III) nitrate (0.236 g, 0.925 mmol) was added to a stirred solution of the title compound of Preparation 10 (0.55 g, 0.462 mmol) in tert-butanol:water (3:1, 20 ml) under nitrogen and the resulting mixture was heated at 75° C. for 3 h. The progress of the reaction was monitored by LC-MS analysis. After completion, the reaction was cooled to room temperature and water (20 ml) added. The resulting mixture was filtered through a micro-filter and the filtrate concentrated under reduced pressure. The residue obtained was triturated with methyl tert-butyl ether (5×3 ml) and diethyl ether (10 ml) to produce a sticky solid which was dried under vacuum to afford the crude tile compound as a pale-yellow solid. The crude title compound was purified by reverse phase HPLC (Mobile phase A: 0.1% Formic acid in water; and Mobile phase B: acetonitrile—column. Inertsil ODS3V 250×20 mm, 5 μm) to produce the title compound as an off-white solid (yield: 78 mg, 13.4%). LC-MS, C₅₇H₇₇FGaN₁₁O₁₄Si calculated 1255.47; observed 1254.40 [M−H]⁻. ¹H NMR (400 MHz. DMSO): δ 9.03 (bs, 1H), 8.80 (d, J=4.0 Hz, 1H), 8.59 (bs, 1H), 8.12 (bs, 2H), 7.97-7.85 (m, 4H), 7.67 (d, J=7.6 Hz, 2H), 7.51 (d, J=4.4 Hz, 1H), 7.46-7.44 (m, 1H), 4.84 (d, J=4.8 Hz, 1H), 4.56 (bs, 1H), 4.47 (bs, 1H), 4.21-4.16 (m, 4H), 3.75 (bs, 1H), 3.56-3.41 (m, 14H), 3.20-2.70 (m, 16H), 2.17 (bs, 1H), 2.07 (bs, 1H), 1.95 (bs, 2H), 1.07 (s, 18H).

Synthesis of Conjugates of Example 3 and Example 4

There is shown in the below scheme an exemplary synthetic procedure to obtain the conjugates of Example 3 and Example 4, as detailed in Table 1.

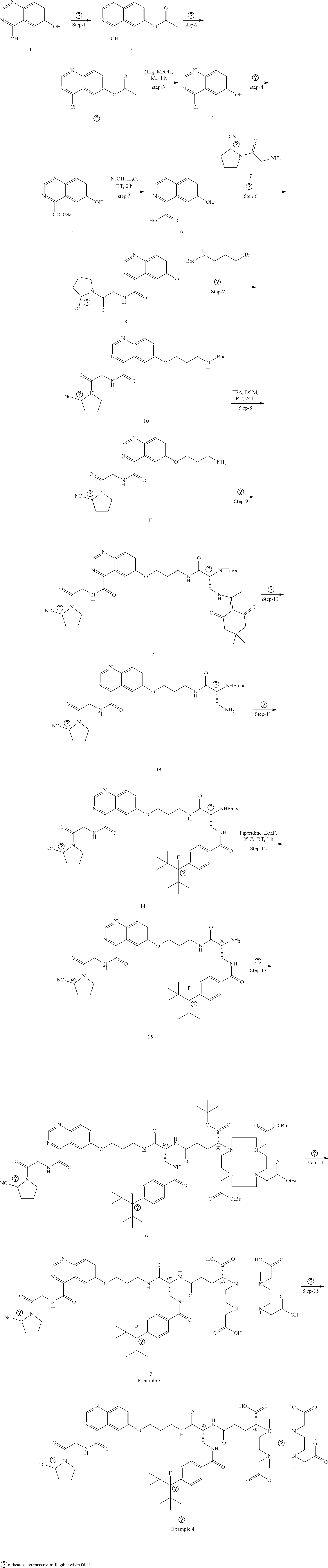

Synthesis of Intermediates

Preparation 1

4-Hydroxyquinazolin-6-yl acetate (2)

A mixture of quinazoline-4,6-diol (5 g, 30.86 mmol; Combi-blocks), acetic anhydride (46.6 mL, 49.38 mmol; Spectrochem) and pyridine (8 mL; Spectrochem) was heated at 100° C. under nitrogen for 2 h. The progress of the reaction was monitored by thin-layer chromatography (TLC) analysis (5% methanol in dichloromethane). After completion, the reaction was cooled to room temperature and the reaction mixture was poured on to crushed ice (50 mL). This resulted in pale-yellow precipitation. The solid was collected by filtration, washed with fresh ice-cold water (200 mL) and dried under vacuum to afford the desired compound as a pale-yellow solid. The title compound was characterized by LC-MS and ¹H NMR analysis. Yield: 6.3 g (100%). LC-MS C₁₀H₈N₂O₃ calculated 204.05; observed 205.25 [M+H]⁺. ¹H NMR (400 MHz, CDCl₃): δ 10.74 (bs, 1H), 8.06-8.02 (m, 2H), 7.80 (d, J=8.8 Hz, 1H), 7.55 (d, J=8.4 Hz, 1H), 2.37 (s, 3H).

Preparation 2

4-Chloroquinazolin-6-yl acetate (3)

To a mixture of 4-hydroxyquinazolin-6-yl acetate (6.3 g, 30.86 mmol) and thionyl chloride (43.05 g, 26.25 mL, 361.8 mmol; Spectrochem) was added catalytic amount of dry N,N-dimethyl formamide (DMF, 0.3 mL) under nitrogen. The resulting mixture was heated at 90° C. for 3 h. The progress of the reaction was monitored by thin-layer chromatography (TLC) analysis (5% methanol in dichloromethane). After completion, the reaction was cooled to room temperature and the reaction mixture concentrated under reduced pressure. The residue was azeotroped with toluene (50 mL) and dried under vacuum to afford the desired compound as a pale-yellow solid. The title compound was characterized by LC-MS and ¹H NMR analysis. Yield: 6.8 g (100%). LC-MS C₁₀H₇ClN₂O₂ calculated 222.02; observed 223.15 [M+H]⁺. ¹H NMR (400 MHz, CDCl₃): δ 9.08 (s, 1H), 8.17 (d, J=9.2 Hz, 1H), 8.04 (s, 1H), 7.76 (d, J=9.2 Hz, 1H), 2.42 (s, 3H).

Preparation 3

4-Chloroquinazolin-6-ol (4)

A mixture of 4-chloroquinazolin-6-yl acetate (19 g, 85.30 mmol) and ammonia (7 N in methanol; 400 mL; Hychem laboratories) was stirred at room temperature for 1 h. The progress of the reaction was monitored by thin-layer chromatography (TLC) analysis (5% methanol in dichloromethane). After completion, the reaction mixture concentrated under reduced pressure. The residue was triturated with diethyl ether (10 mL) and dried under vacuum to afford the desired compound as a dark brown solid. It was taken in next step without further purification. The title compound was characterized by LC-MS analysis. Yield: 13.3 g (86.6%). LC-MS C₈H₅ClN₂O calculated 180.01; observed: 181.20 [M+H]⁺.

Preparation 4

Methyl 6-hydroxyquinazoline-4-carboxylate (5)

A stirred solution of 4-chloroquinazolin-6-ol (13.3 g, 73.6 mmol), triethyl amine (30.6 mL, 220.9 mmol; Spectrochem) in dry methanol (200 mL) was purged with nitrogen for 20 min at room temperature. To this was added PdCl₂dppf (5.3 g, 7.36 mmol; Chempure) and the resulting mixture was heated at 60° C. under CO pressure (5 kg). The progress of the reaction was monitored by thin-layer chromatography (TLC) analysis (5% methanol in dichloromethane). After completion, the reaction mixture was cooled to room temperature and filtered through a celite bed. The filtrated was concentrated under reduced pressure to get crude compound. The crude was subjected to silica gel (100-200) column chromatography using 10-50% ethyl acetate in n-hexane to afford the desired compound as a pale-yellow solid. The title compound was characterized by LC-MS and ¹H NMR analysis. Yield: 8.6 g (57.3%). LC-MS C₁₀H₈N₂O₃ calculated 204.05; observed 205.25 [M+H]⁺. ¹H NMR (400 MHz, DMSO): δ 10.76 (s, 1H), 9.21 (s, 1H), 8.01 (d, J=9.2 Hz, 1H), 7.72 (s, 1H), 7.66 (d, J=9.2 Hz, 1H), 4.02 (s, 3H).

Preparation 5

6-Hydroxyquinazoline-4-carboxylic acid (6)

To a stirred solution of methyl 6-hydroxyquinazoline-4-carboxylate (8.6 g, 42.1 mmol) in tetrahydrofuran:methanol:water (6:1:0.5; 75 mL) was added sodium hydroxide (4.2 g, 105.3 mmol) and the mixture was stirred at room temperature for 2 h. The progress of the reaction was monitored by TLC analysis (5% methanol in dichloromethane). After completion, the reaction mixture was concentrated under reduced pressure to one third of the volume. The pH of the solution was adjusted to 7 using concentrated hydrochloric acid (HCl). Initially formed solid was removed by filtration and the filtrated was further acidified to pH=1 using concentrated HCl. The yellow solid formed was collected by filtration, washed with fresh water, and dried under vacuum to afford the desired compound as a pale-yellow solid. The title compound was characterized by LC-MS and ¹H NMR analysis. Yield: 5.3 g (66.6%). LC-MS C₉H₆N₂O₃ calculated 190.04; observed 191.20 [M+H]⁺. ¹H NMR (400 MHz, DMSO): δ 14.12 (bs, 1H), 10.69 (s, 1H), 9.19 (s, 1H), 7.99 (d, J=9.2 Hz, 1H), 7.72 (s, 1H), 7.64 (d, J=9.6 Hz, 1H).

Preparation 6

(S)—N-(2-(2-Cyanopyrrolidin-1-yl)-2-oxoethyl)-6-hydroxyquinazoline-4-carboxamide (8)

To a stirred solution of HBTU (9.09 g, 24.0 mmol; Spectrochem) in dry N,N-dimethyl formamide (DMF, 70 mL) were added 6-hydroxyquinazoline-4-carboxylic acid (3.8 g, 20.0 mmol), hydroxybenzotriazole (HOBt, 6.1 g, 40.0 mmol; Spectrochem) and diisopropyl ethyl amine (8.9 mL, 50.0 mmol; Spectrochem). This was followed by an addition of (S)-1-glycylpyrrolidine-2-carbonitrile (4.8 g, 30.0 mmol) and the resulting mixture was stirred at room temperature for 16 h. The progress of the reaction was monitored by thin-layer chromatography (TLC) analysis (10% methanol in dichloromethane). After completion of the reaction, water (100 mL) was added, and the mixture was extracted with ethyl acetate (500 mL×3). The combined organic layer was given brine (200 mL) wash and dried over anhydrous sodium sulphate. The solvent was removed under reduced pressure and the crude compound was subject to silica gel (230-400) column chromatography using 5-10% methanol in dichloromethane to afford the desired compound as a pale-yellow solid. The title compound was characterized by LC-MS analysis. Yield: 3.2 g (49.0%). LC-MS C₁₆H₁₅N₅O₃ calculated 325.12, observed 326.05 [M+H]⁺.

Preparation 7

tert-Butyl (S)-(3-((4-((2-(2-cyanopyrrolidin-1-yl)-2-oxoethyl)carbamoyl)quinazolin-6-yl)oxy)propyl)carbamate (10)

To a solution of (S)—N-(2-(2-cyanopyrrolidin-1-yl)-2-oxoethyl)-6-hydroxyquinazoline-4-carboxamide (3.2 g, 9.8 mmol) in dry DMF (32 mL) under nitrogen was added K₂CO₃ (1.6 g, 11.81 mmol; Spectrochem) and tert-butyl (3-bromopropyl)carbamate (2.8 g, 11.81 mmol; BLD-pharma). The resulting mixture was stirred at 60° C. for 24 h. The progress of the reaction was monitored by TLC analysis (10% methanol in dichloromethane). After completion, the reaction was cooled to room temperature and water (100 mL) was added. The resulting mixture was extracted with ethyl acetate (200 mL×3). The combined organic layer was given brine wash and dried over anhydrous sodium sulphate. The solution was filtered and concentrated under reduced pressure. The crude compound was purified by flash silica-gel (230-400) column chromatography using 5-6% methanol in dichloromethane to afford the desired compound as a pale-yellow solid. The desired compound was characterized by LC-MS and ¹H NMR analysis. Yield: 3.4 g (72.0%). LC-MS C₂₄H₃₀N₆O₅ calculated 482.23; observed 481.15 [M−H]⁻. ¹H NMR (400 MHz. DMSO): δ 9.30-9.28 (m, 2H), 8.44 (s, 1H), 8.03 (d, J=9.6 Hz, 1H), 7.73 (d. J=8.8 Hz, 1H), 6.96 (s, 1H), 4.83 (bs, 1H), 4.25 (d, J=5.2 Hz, 2H), 4.15 (s, 2H), 3.74 (bs, 1H), 3.58-3.54 (m, 1H), 3.14 (d, J=5.6 Hz, 2H), 2.19-2.07 (m, 4H), 1.95-1.92 (m, 2H), 1.37 (s, 9H).

Preparation 8

(S)-6-(3-aminopropoxy)-N-(2-(2-cyanopyrrolidin-1-yl)-2-oxoethyl)quinazoline-4-carboxamide (11)

To a solution of tert-butyl (S)-(3-((4-((2-(2-cyanopyrrolidin-1-yl)-2-oxoethyl)carbamoyl)quinazolin-6-yl)oxy)propyl)carbamate (3.4 g, 7.0 mmol) in dichloromethane (DCM, 34 mL) under inert atmosphere added trifluoracetic acid (2.1 mL, 28.2 mmol; Spectrochem). The resulting mixture was stirred at room temperature for 24 h. The progress of the reaction was monitored by thin-layer chromatography (TLC) analysis (15% methanol in dichloromethane). After completion, the reaction mixture was concentrated under reduced pressure and the residue was co-distilled with fresh dichloromethane (20 mL×3). Finally, the residue was triturated with diethyl ether (20 mL) and dried under vacuo to afford the desired compound as a light brown solid in quantitative yield. The crude compound was taken for the next step without further purifications. The desired compound was characterized by LC-MS analysis. LC-MS C₁₉H₂₂N₆O₃ calculated 382.18; observed 383.35 [M+H]⁺.

Preparation 9

(9H-Fluoren-9-yl)methyl ((R)-1-((3-((4-((2-((S)-2-cyanopyrrolidin-1-yl)-2-oxoethyl)carbamoyl)quinazolin-6-yl)oxy)propyl)amino)-3-((1-(4,4-dimethyl-2,6-dioxocyclohexylidene)ethyl)amino)-1-oxopropan-2-yl)carbamate (12)

To stirred solution of (S)-6-(3-aminopropoxy)-N-(2-(2-cyanopyrrolidin-1-yl)-2-oxoethyl)quinazoline-4-carboxamide (1.8 g, 4.71 mmol) and Fmoc-(n-beta-1-(4,4-dimethyl-2,6-dioxocyclohex-1-ylidene)ethyl)-I-alpha, beta-diamino propionic acid (2.5 g, 5.1 mmol; Argonix Reagents & Intermediates) in dry N,N-dimethylformamide (DMF, 20 mL) were added 2,4,6-collidine (4.2 mL, 31.4 mmol; Spectrochem), 1-hydroxy-7-azabenzotriazole (HOAt, 1.2 g, 9.4 mmol; Spectrochem) and 2-(1H-benzotriazole-1-yl)-1,1,3,3-tetramethylaminium tetrafluoroborate (TBTU, 3.0 g, 9.4 mmol; Spectrochem) under nitrogen. The resulting mixture was stirred at room temperature for 16 h. The progress of the reaction was monitored by thin-layer chromatography (TLC) analysis (15% methanol in dichloromethane). After completion of the reaction, ice cold water (100 mL) was added, and the resulting mixture was extracted with ethyl acetate (200 mL×3). The combined organic layer was dried over anhydrous sodium sulphate and concentrated under reduced pressure. The crude compound was purified by silica-gel (230-400 silica) column chromatography using 5-10% methanol in dichloromethane to afford the desired compound as an off-white solid. The desired compound was characterized by LC-MS and ¹H NMR analysis. Yield: 1.3 g (30%). %). LC-MS C₄₇H₅₀N₈O₈ calculated 854.38; observed 855.65 [M+H]⁺. ¹H NMR (400 MHz, DMSO): δ 13.48 (d. J=7.6 Hz, 1H), 9.27 (s, 1H), 8.55-8.46 (m, 2H), 7.99 (d, J=8.8 Hz, 1H), 7.86 (d, J=7.6 Hz, 2H), 7.71-7.62 (m, 4H), 7.40-7.37 (m, 2H), 7.30-7.28 (m, 2H), 4.81 (bs, 1H), 4.58 (bs, 1H), 4.28-4.18 (m, 8H), 3.71 (bs, 1H), 3.53-3.51 (m, 1H), 3.37 (bs, 1H), 2.46 (s, 3H), 2.22-1.98 (m, 11H), 1.09 (t, J=6.4 Hz, 2H), 0.91 (s, 6H).

Preparation 10

(9H-Fluoren-9-yl)methyl ((R)-3-amino-1-((3-((4-((2-((S)-2-cyanopyrrolidin-1-yl)-2-oxoethyl)carbamoyl)quinazolin-6-yl)oxy)propyl)amino)-1-oxopropan-2-yl)carbamate (13)

To a stirred solution of (9H-fluoren-9-yl)methyl ((R)-1-((3-((4-((2-((S)-2-cyanopyrrolidin-1-yl)-2-oxoethyl)carbamoyl)quinazolin-6-yl)oxy)propyl)amino)-3-((1-(4,4-dimethyl-2,6-dioxocyclohexylidene)ethyl)amino)-1-oxopropan-2-yl)carbamate (0.62 g, 0.65 mmol) in ethanol (6 mL) was added hydrazine hydrate (25%, 0.3 mL; Spectrochem). The resulting mixture was stirred at room temperature for 2 h. The progress of the reaction was monitored by thin-layer chromatography (TLC) analysis (15% methanol in dichloromethane). After completion of the reaction, water (30 mL) was added, and the resulting mixture was extracted with dichloromethane (100 mL×3). The combined organic layer was dried over anhydrous sodium sulphate and concentrated under reduced pressure. The crude compound was purified by silica-gel (100-200 silica) column chromatography using 5-10% methanol in dichloromethane to afford the desired compound as an off-white solid. The desired compound was characterized by LC-MS and ¹H NMR analysis. Yield: 0.42 g (100%). LC-MS C₃₇H₃₈N₈O₈ calculated 690.29; observed 691.50 [M+H]⁺. ¹H NMR (400 MHz,): δ 9.29-9.27 (m, 2H), 8.45 (s, 1H), 8.11 (bs, 1H), 8.01 (d, J=8.8 Hz, 1H), 7.87 (d, J=7.2 Hz, 2H), 7.73-7.66 (m, 3H), 7.40-7.31 (m, 4H), 4.82 (bs, 1H), 4.27-4.17 (m, 8H), 3.72 (bs, 1H), 3.53-3.52 (m, 1H), 3.29-3.27 (m, 4H), 3.02 (bs, 1H), 2.17-1.91 (m, 6H), 1.09 (t, J=6.0 Hz, 2H).

Preparation 11

(9H-Fluoren-9-yl)methyl ((R)-1-((3-((4-((2-((S)-2-cyanopyrrolidin-1-yl)-2-oxoethyl)carbamoyl)quinazolin-6-yl)oxy)propyl)amino)-3-(4-(di-tert-butylfluorosilyl)benzamido)-1-oxopropan-2-yl)carbamate (14) (Example 13)

To stirred solution of (9H-fluoren-9-yl)methyl ((R)-3-amino-1-((3-((4-((2-((S)-2-cyanopyrrolidin-1-yl)-2-oxoethyl)carbamoyl)quinazolin-6-yl)oxy)propyl)amino)-1-oxopropan-2-yl)carbamate (0.42 g, 0.61 mmol) and 4-(di-tert-butylfluorosilyl)benzoic acid (SiFA-BA, 0.25 g, 0.89 mmol) in dry N,N-dimethylformamide (DMF, 5 mL) were added N,N-diisopropylethylamine (DIPEA, 0.72 mL, 4.0 mmol; Spectrochem), 1-hydroxy-7-azabenzotriazole (0.122 g, 0.9 mmol; Spectrochem) and 2-(1H-benzotriazole-1-yl)-1,1,3,3-tetramethylaminium tetrafluoroborate (TBTU, 0.28 g, 0.9 mmol; Spectrochem) under nitrogen. The resulting mixture was stirred at room temperature for 16 h. The progress of the reaction was monitored by TLC analysis (10% methanol in dichloromethane). After completion of the reaction, water (30 mL) was added, and the resulting mixture was extracted with ethyl acetate (100 mL×2). The combined organic layer was dried over anhydrous sodium sulphate and concentrated under reduced pressure. The crude compound was purified by silica-gel (230-400 silica) column chromatography using 1-10% methanol in dichloromethane to afford the desired compound as an off-white solid. The desired compound was characterized by LC-MS analysis. Yield: 0.4 g (70%) LC-MS C₅₂H₅₉FN₈O₇Si calculated 954.43; observed 955.65 [M+H]⁺.

Preparation 12

6-(3-((R)-2-amino-3-(4-(di-tert-butylfluorosilyl)benzamido)propanamido)propoxy)-N-(2-((S)-2-cyanopyrrolidin-1-yl)-2-oxoethyl)quinazoline-4-carboxamide (15)

To a stirred solution of (9H-fluoren-9-yl)methyl ((R)-1-((3-((4-((2-((S)-2-cyanopyrrolidin-1-yl)-2-oxoethyl)carbamoyl)quinazolin-6-yl)oxy)propyl)amino)-3-(4-(di-tert-butylfluorosilyl)benzamido)-1-oxopropan-2-yl)carbamate (0.4 g, 0.41 mmol) in dimethylformamide (DMF, 3 mL) was dropwise added a solution of piperidine (0.04 mL, 0.50 mmol; Spectrochem) in N,N-dimethylformamide (1 mL). The resulting mixture was stirred at room temperature for 1 h. The progress of the reaction was monitored by thin-layer chromatography (TLC) analysis (15% methanol in dichloromethane). After completion of the reaction, water (50 mL) was added, and the resulting mixture was extracted with ethyl acetate (70 mL×3). The combined organic layer was dried over anhydrous sodium sulphate and concentrated under reduced pressure. The crude compound was purified by silica-gel (230-400 silica) column chromatography using 5-12% methanol in dichloromethane to afford the desired compound as an off-white solid. The desired compound was characterized by LC-MS and ¹H NMR analysis. Yield: 0.22 g (73%). LC-MS C₃₇H₄₉FN₈O₅Si calculated 732.36; observed 733.65 [M+H]⁺. ¹H NMR (400 MHz. DMSO): δ 9.31-9.26 (m, 2H), 8.84 (bs, 1H), 8.45 (s, 1H), 8.30 (s, 1H), 8.01 (d, J=7.6 Hz, 3H), 7.74-7.64 (m, 3H), 7.10 (bs, 3H), 4.82 (bs, 1H), 4.66 (bs, 1H), 4.25-4.13 (m, 4H), 3.73 (bs, 1H), 3.53-3.52 (m, 1H), 3.35-3.09 (m, 3H), 2.50-2.07 (m, 6H), 1.23 (s, 18H).

Preparation 13

tri-tert-Butyl 2,2′,2″-(10-((R)-1-(tert-butoxy)-5-(((R)-1-((3-((4-((2-((S)-2-cyanopyrrolidin-1-yl)-2-oxoethyl)carbamoyl)quinazolin-6-yl)oxy)propyl)amino)-3-(4-(di-tert-butylfluorosilyl)benzamido)-1-oxopropan-2-yl)amino)-1,5-dioxopentan-2-yl)-1,4,7,10-tetraazacyclododecane-1,4,7-triyl)triacetate (16)

To a stirred solution of 6-(3-((R)-2-amino-3-(4-(di-tert-butylfluorosilyl)benzamido)propanamido)propoxy)-N-(2-((S)-2-cyanopyrrolidin-1-yl)-2-oxoethyl)quinazoline-4-carboxamide (0.22 g, 0.30 mmol) and 1,4,7,10-tetraazacyclodocecane, 1-(glutaric acid)-4,7,10-triacetic acid (DOTAGA(tBu)₄,0.21 g, 0.30 mmol; Argonix Reagents & Intermediates) in dichloromethane (4 mL) was added N,N-diisopropylethylamine (DIPEA, 0.16 mL, 0.90 mmol; Spectrochem) under nitrogen. This was followed by an addition of 1-propanephosphonic anhydride (50% in ethyl acetate, 0.28 mL, 0.90 mmol; Spectrochem). The resulting mixture was stirred at room temperature for 3 h. The progress of the reaction was monitored by TLC analysis (15% methanol in dichloromethane). After completion of the reaction, water (30 mL) was added, and the resulting mixture was extracted with dichloromethane (50 mL×3). The combined organic layer was dried over anhydrous sodium sulphate and concentrated under reduced pressure. The crude compound was purified by silica-gel (230-400 silica) column chromatography using 10-15% methanol in dichloromethane to afford the desired compound as an off-white solid. The desired compound was characterized by LC-MS and ¹H NMR analysis. Yield: 0.29 g (69.0%). %). LC-MS C₇₂H₁₁₁FN₁₂O₁₄Si calculated 1414.81, observed 1413.85 [M−H]⁻. ¹H NMR (400 MHz, DMSO): δ 9.31-9.28 (m, 2H), 8.56-8.47 (m, 2H), 8.16 (bs, 1H), 8.09 (bs, 1H), 8.02-7.90 (m, 3H), 7.73-7.63 (m, 3H), 4.81 (bs, 1H), 4.47 (bs, 1H), 4.24-4.13 (m, 5H), 3.72 (bs, 1H), 3.54-3.52 (m, 2H), 3.30 (bs, 3H), 3.16 (d, J=5.2 Hz, 2H), 3.03-3.00 (m, 5H), 2.85-2.70 (m, 4H), 2.33-1.83 (m, 21H), 1.40 (s, 36H), 1.08 (s, 18H).

Synthesis of Conjugates of Examples 3 and 4

Preparation 14—Compound of Example 3

2,2′,2″-(10-((R)-1-carboxy-4-(((R)-1-((3-((4-((2-((S)-2-cyanopyrrolidin-1-yl)-2-oxoethyl)carbamoyl)quinazolin-6-yl)oxy)propyl)amino)-3-(4-(di-tert-butylfluorosilyl)benzamido)-1-oxopropan-2-yl)amino)-4-oxobutyl)-1,4,7,10-tetraazacyclododecane-1,4,7-triyl)triacetic acid (17)

A solution of tri-tert-butyl 2,2′,2″-(10-((R)-1-(tert-butoxy)-5-(((R)-1-((3-((4-((2-((S)-2-cyanopyrrolidin-1-yl)-2-oxoethyl)carbamoyl)quinazolin-6-yl)oxy)propyl)amino)-3-(4-(di-tert-butylfluorosilyl)benzamido)-1-oxopropan-2-yl)amino)-1,5-dioxopentan-2-yl)-1,4,7,10-tetraazacyclododecane-1,4,7-triyl)triacetate (0.29 g, 0.20 mmol) in trifluoroacetic acid (TFA): triisopropylsilane (TIS):water (95:2.5:2.5, 22 mL; Spectrochem) was stirred at room temperature for 36 h under nitrogen. The progress of the reaction was monitored by LC-MS analysis. After completion of the reaction, the reaction mixture was concentrated under reduced pressure to obtain sticky liquid. The residue was triturated with methyl tert-butyl ether (20 mL×3) followed by with n-pentane (10 mL) resulted in solid formation. The solid was dried under vacuum to afford the desired compound as a light brown solid. The desired compound was characterized by LC-MS analysis. Yield: 0.19 g (77.8%, crude). LC-MS C₅₆H₇₉FN₁₂O₁₄Si calculated 1191.56; observed 1189.70 [M−H]⁻.

Preparation 15—Compound of Example 4

Gallium(I) 2-(16-((R)-1-carboxy-4-(((R)-1-((3-((4-((2-((S)-2-cyanopyrrolidin-1-yl)-2-oxoethyl)carbamoyl)quinazolin-6-yl)oxy)propyl)amino)-3-(4-(di-tert-butylfluorosilyl)benzamido)-1-oxopropan-2-yl)amino)-4-oxobutyl)-3,6-dioxo-4,5-dioxa-1,8,11,16-tetraazabicyclo[6.5.5]octadecan-11-yl)acetate (18, Ga—(S,R,R)—SiFA-FAP-2)

To a stirred solution of 2,2′,2″-(10-((R)-1-carboxy-4-(((R)-1-((3-((4-((2-((S)-2-cyanopyrrolidin-1-yl)-2-oxoethyl)carbamoyl)quinazolin-6-yl)oxy)propyl)amino)-3-(4-(di-tert-butylfluorosilyl)benzamido)-1-oxopropan-2-yl)amino)-4-oxobutyl)-1,4,7,10-tetraazacyclododecane-1,4,7-triyl)triacetic acid (crude, 0.19 g, 0.16 mmol) in tert-butanol: water (3:1, 7.3 mL) under nitrogen was added gallium (III) nitrate (0.082 g, 0.32 mmol; Sigma Aldrich) and the resulting mixture was heated at 75° C. for 3 h. The progress of the reaction was monitored by LC-MS analysis. After completion, the reaction was cooled to room temperature and added water (10 mL). The resulting mixture was filtered through micro filter and the filtrate was concentrated under reduced pressure. The residue obtained was triturated with methyl tert-butyl ether (10 mL×3) and diethyl ether (10 mL). The sticky residue was dried under high vacuum to afford the pale-yellow solid. The crude compound was purified by reverse phase HPLC; Mobile phase A: A: 0.1% Formic acid in water and Mobile phase B: Acetonitrile; Column: Waters XBridge Prep C18 250×19 mm, 5 μm to afford the desired compound as an off-white solid. Yield: 13 mg (6.5%). LC-MS C₅₆H₇₆FGaN₁₂O₁₄Si calculated 1256.46; observed 1257.80 [M+H]⁺. ¹H NMR (400 MHz, DMSO): δ 9.33-9.27 (m, 2H), 8.61 (m, 1H), 8.46 (bs, 1H), 8.16 (bs, 2H), 8.02-7.91 (m, 3H), 7.75-7.65 (m, 3H), 4.84 (bs, 1H), 4.57 (bs, 1H), 4.45 (bs, 1H), 4.25-4.17 (m, 4H), 3.73 (bs, 1H), 3.48 (m, 4H), 3.30-2.70 (m, 23H), 2.18-1.83 (m, 11H), 1.02 (s, 18H); HPLC: 7.730 min; 90.6%, Column: XBridge C18 250×4.6, 5 μm, Mobile phase A: 0.1% formic acid in H₂O; Mobile phase B: Acetonitrile.

Synthesis of conjugates of Examples 5 to 10

The conjugates of Examples 5 to 10 may be prepared in accordance with the below schemes.

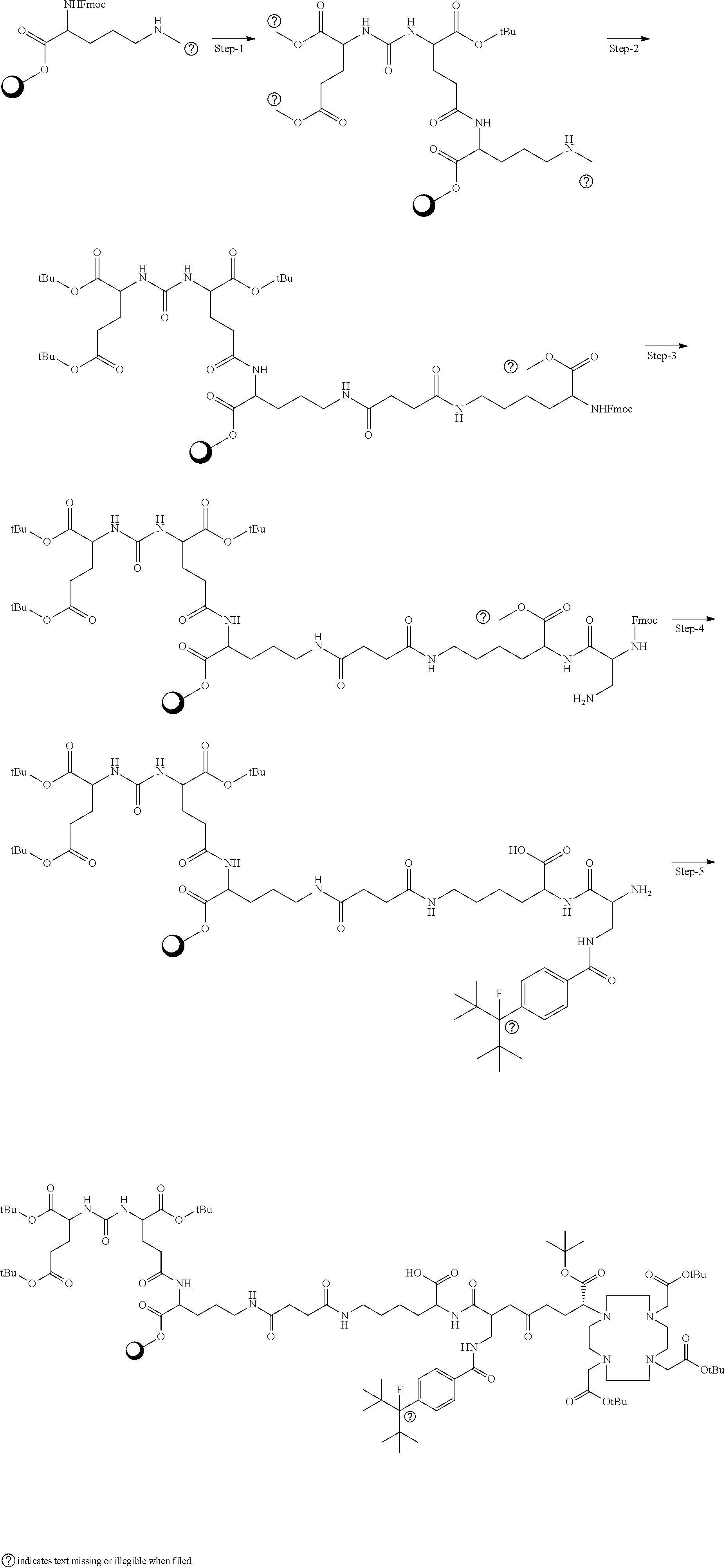

Steps 1-5 above can be conducted according to published methods (WO2019/020831): Step 1: a) 20% piperidine, (DMF); b) (tBuO)EuE(OtBu)₂, HOBt, TBTU, DIPEA, (DMF); Step 2: a) 2% hydrazine (DMF); b) succinic anhydride, DIPEA, (DMF); c) Fmoc-D-Lys-OAII-HCl, HOBt, TBTU, DIPEA, (DMF); Step 3: a) 20% piperidine, (DMF); b) Fmoc-D-Dap (Dde)-OH, HOBt, TBTU, DIPEA, (DMF); c) imidazole, hydroxylamine hydrochloride, (NMP, DMF); Step 4: a) SIFA-BA (WO2019/020831), HOBt, TBTU, DIPEA (DMF); b) 20% piperidine, (DMF); Step 5: DOTAGA anhydride, DIPEA, (DMF)

Example 5

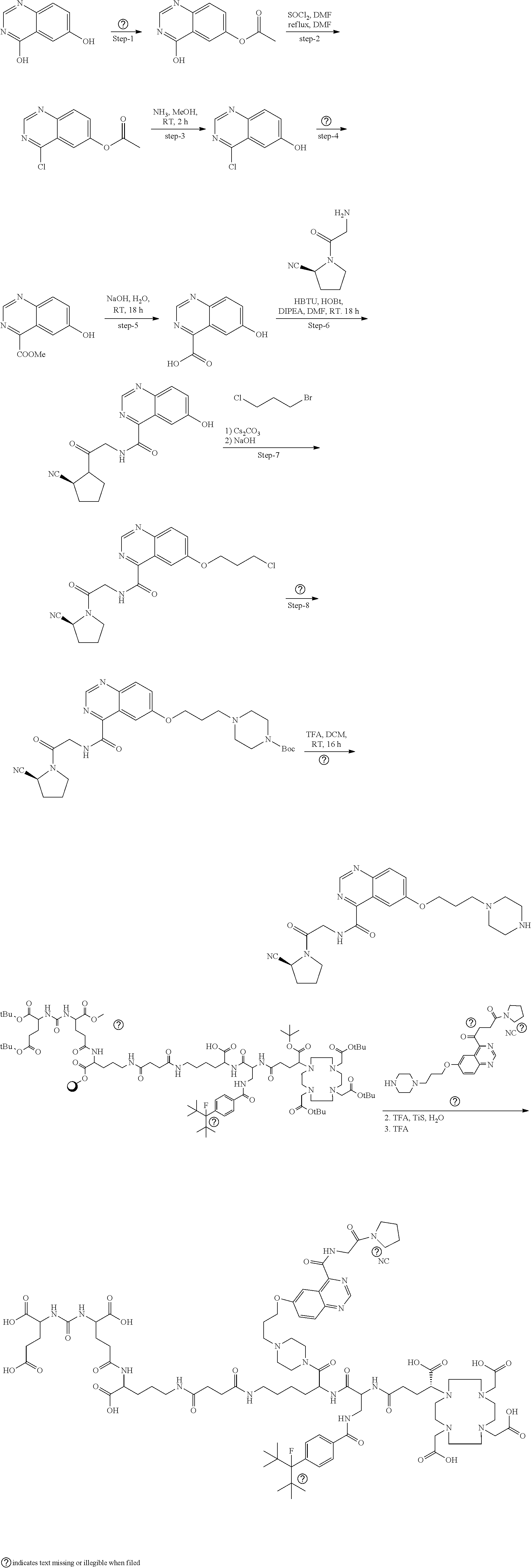

Example 6

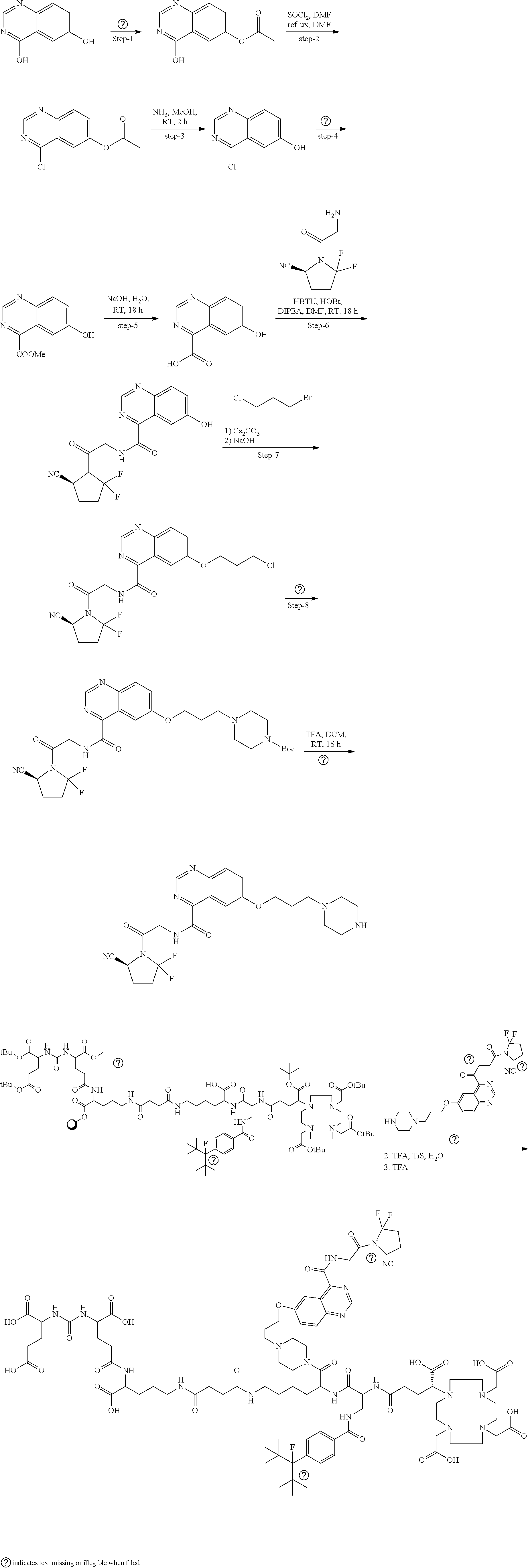

Example 7

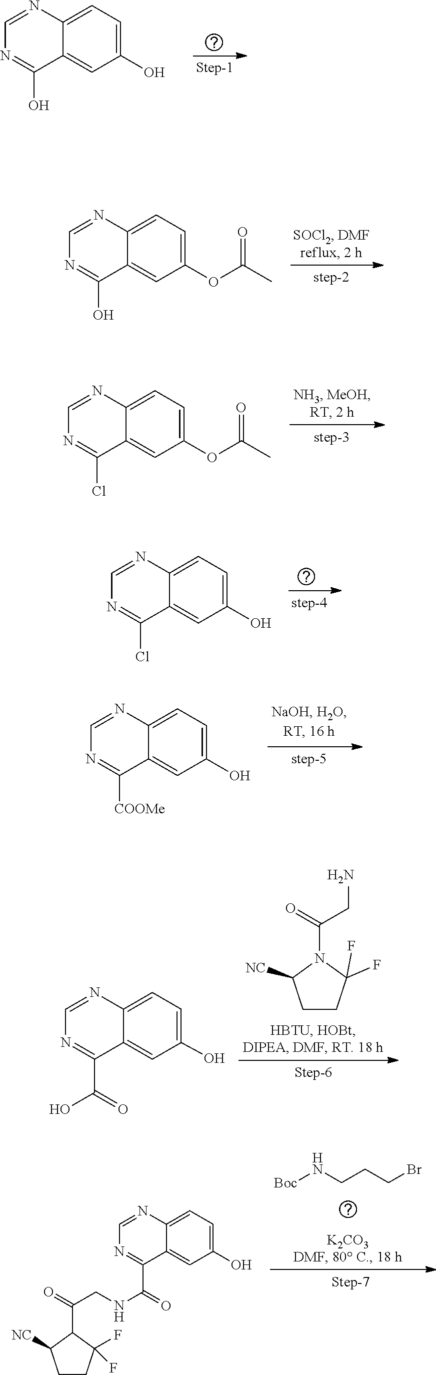

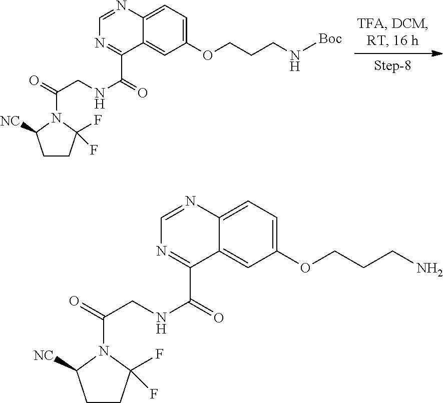 [SMILES: CC(C)(C)OC(=O)NCCCOc1ccc2ncnc(C(=O)NCC(=O)N3[C@H](C#N)CCC3(F)F)c2c1 ; N#C[C@@H]1CCC(F)(F)N1C(=O)CNC(=O)c1ncnc2ccc(OCCCN)cc12]

Synthesis of (S)-1-(2-aminoacetyl)-4,4-difluoropyrrolidine-2-carbonitrile (step 6 above) is conducted according to the method described by Jansen et al. (ACS Med. Chem. Lett. 2013, 4, 491-496).

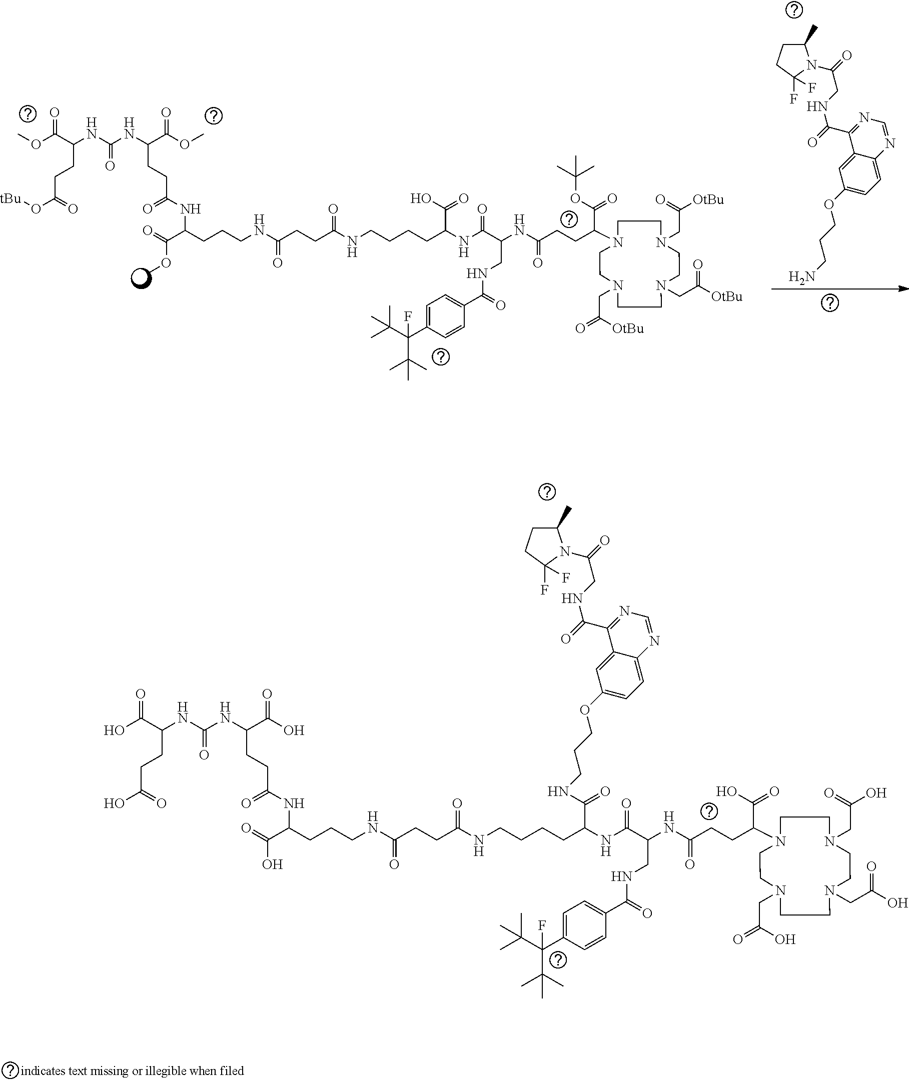

Step 6: a) HATU, DIPEA, (DMF); b) cleavage: TFA TIS, water; c) Final deprotection: TFA.

Example 8 & 8a

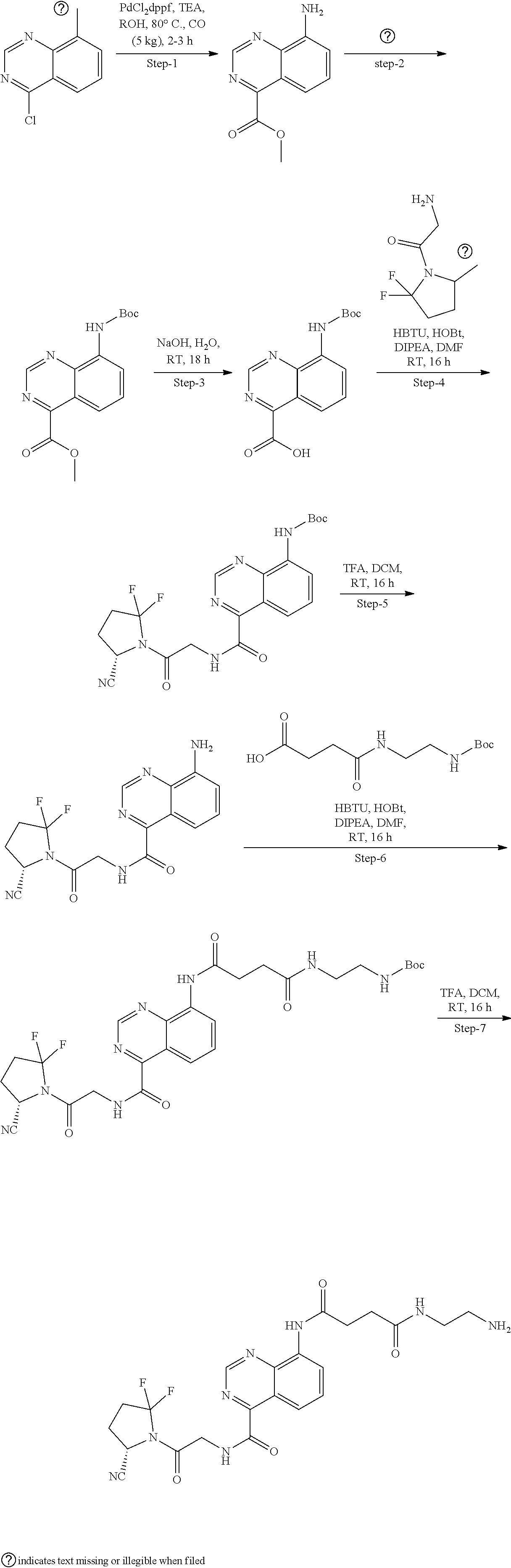

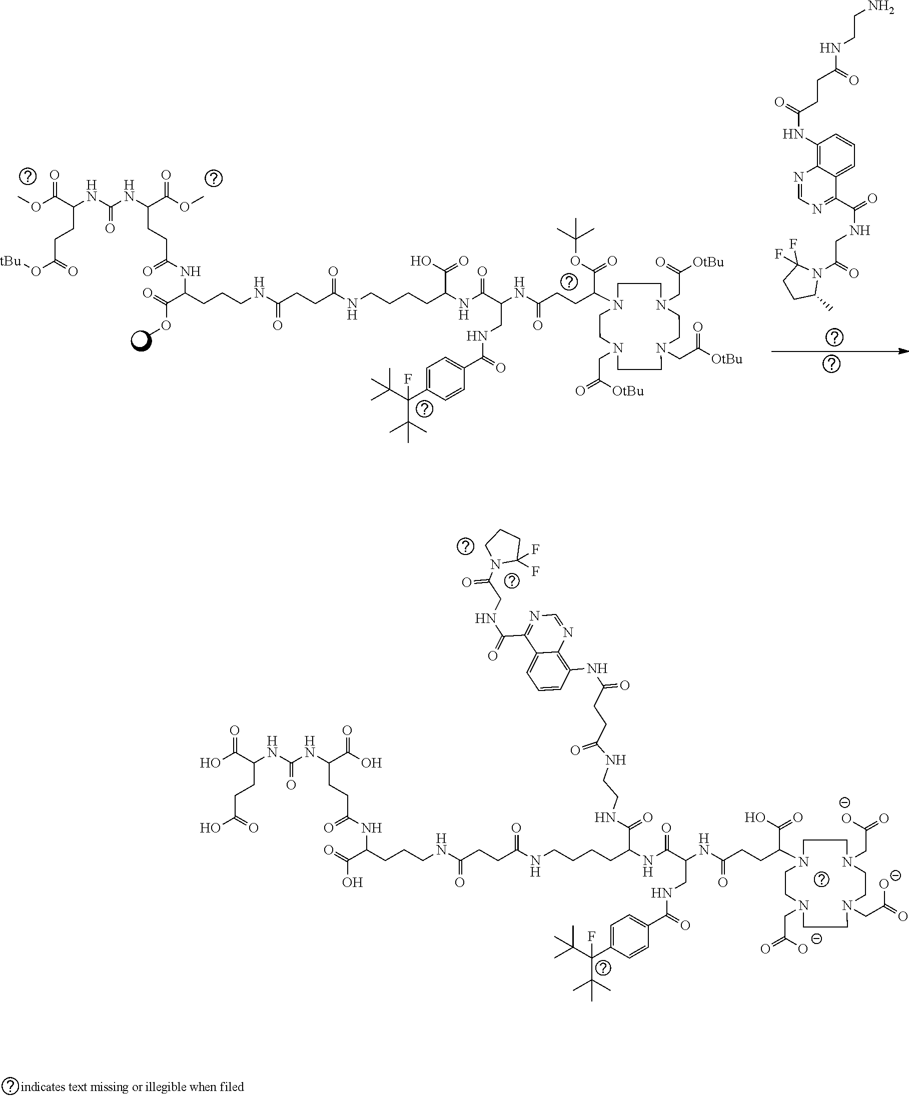

Example 9& 10

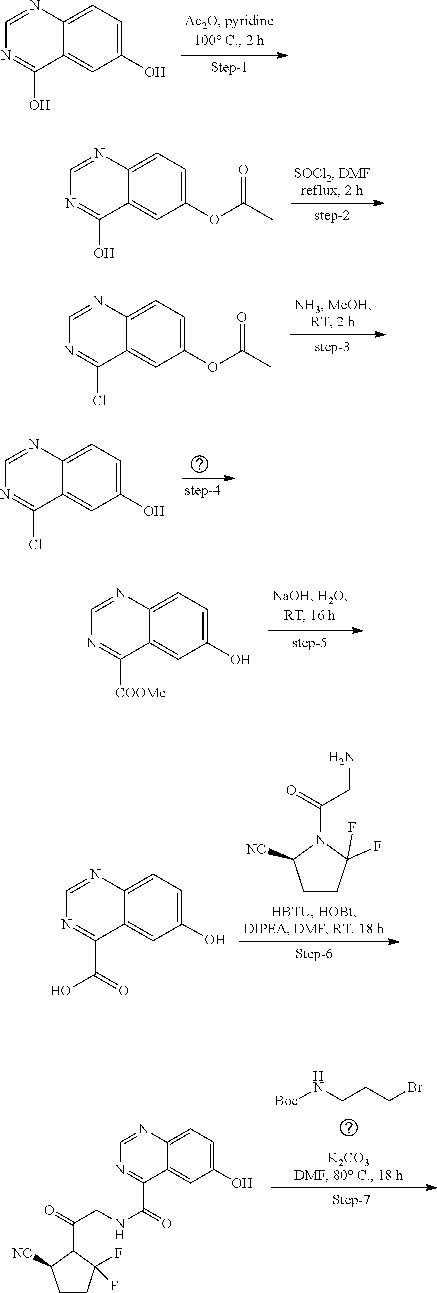

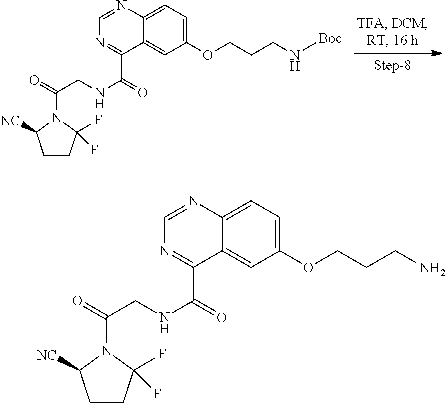 [SMILES: CC(C)(C)OC(=O)NCCCOc1ccc2ncnc(C(=O)NCC(=O)N3[C@H](C#N)CCC3(F)F)c2c1 ; N#C[C@@H]1CCC(F)(F)N1C(=O)CNC(=O)c1ncnc2ccc(OCCCN)cc12]

Synthesis of(S)-1-(2-aminoacetyl)-4,4-difluoropyrrolidine-2-carbonitrile (step 6) is conducted according to the method described by Janson et al. (ACS Med. Chem. Lett. 2013, 4, 491-496).

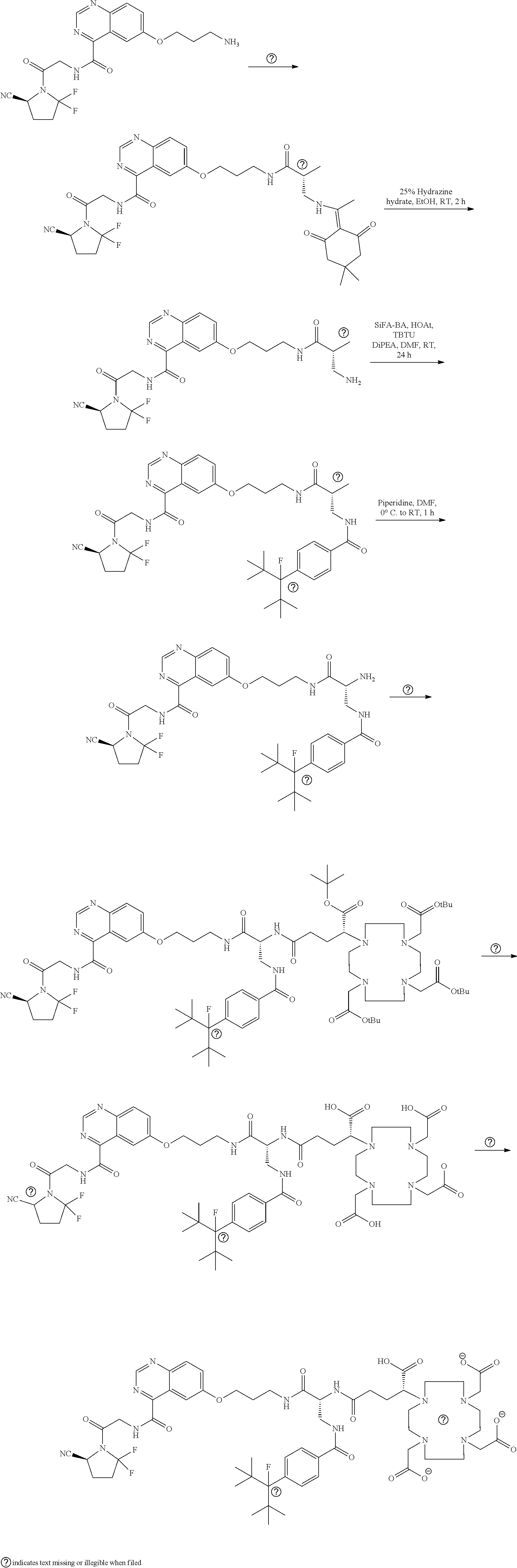

### Radiolabelling

### General Information

No-carrier-added fluorine-18 was produced by Curium Pharma, via the [¹⁸O (p, n)¹⁸F] nuclear reaction by irradiation of a 2.8 mL>97%-enriched [¹⁸O]H₂O target (Bruce Technology) on a PETtrace cyclotron (16 MeV proton beam, GE healthcare). Radio-instant thin-layer chromatography (radio-ITLC) analyses were measured on a mini GITA Dual radio-TLC instrument (Elysia-Raytest) using silica gel-impregnated chromatography paper (Varian Inc.) eluted for 4 minutes with an aqueous Na₂CO₃ 0.1 M solution. Analytical HPLC measurements were performed on a system consisting of an Agilent HP series 1100 (Hewlett Packard, Les Ulis, France) combined with a Flo-one A500 Radiomatic detector (Packard, Canberra, Australia). The separation was carried out on a C-18 column (Kinetex® EVO C18, 5 μm, 4.6×150 mm, 100 Å, equipped with a guard column) using the following solvent conditions: water containing 0.1% of trifluoroacetic acid (solvent A) and acetonitrile containing 0.1% of trifluoroacetic acid (solvent B); 0 to 10 min: gradient elution 99%-0% A with a flow rate of 1 ml/min, λ=254 and 214 nm. Preparation of anhydrous the K[¹⁸F]F-K222-carbonate complex was performed using a SynChrom R&D EVOI synthesis module (Raytest). Oasis HLB Plus LP cartridge (60 μm), Sep-Pak® Light Accell Plus QMA carbonate cartridges (46 mg, 45 μm) and Sep-Pak® light C18 Plus cartridges (130 mg, 55-105 μm) were purchased from Waters.

All radiolabelled compounds were compared by TLC or analytical HPLC to the authentic non-radioactive material and to be free of significant UV-absorbing chemical and radiochemical impurities.

Radiosynthesis of Example 11 ([¹⁸F]—Ga—(S,R,R)—SiFA-FAP-1)

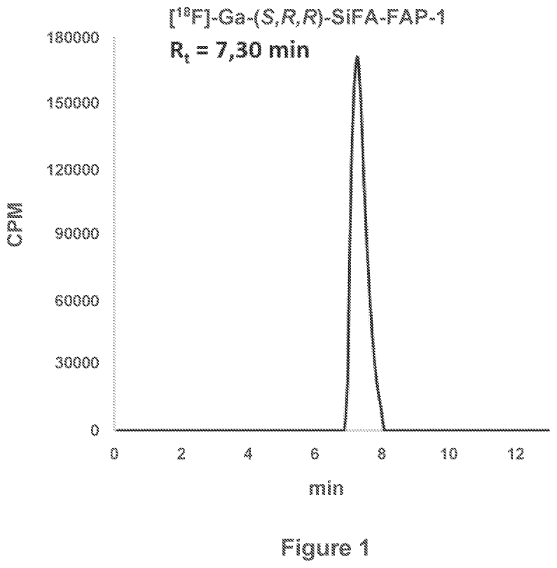

On a SynChrom R&D EVOI synthesis module, the aqueous solution of [¹⁸F]F— in [¹⁸O]H₂O (2.67 GBq at 12h16) was passed through an anion exchange resin (Sep-Pak® Light Accell Plus QMA carbonate cartridge 46 mg) preconditioned with deionised water (10 mL) and air (10 mL). Then a solution of potassium carbonate (2.8 mg) and Kryptofix (K222, 21 mg) in a mixture of water (200 μL) and acetonitrile (700 μL) was passed through the cartridge to elute the radioactivity to the reactor. After 30 s of helium bubbling, the azeotropic drying of the mixture was performed under vacuum and helium flow at 100° C. for 3 minutes. After cooling to 30° C., acetonitrile (1 mL) was added to the reactor. After 30 s of helium bubbling, the reaction mixture was evaporated to dryness under vacuum and helium flow at 110° C. for 3 min. After cooling to 30° C., a freshly prepared solution of the compound of Example 2 (66 μg, 52 nmol) and acetic acid (9 μL) in anhydrous DMSO (500 μL) was added to the dry reactor. The solution was stirred for 10 s and transferred in a closed glass vial containing a magnetic stirrer. The reaction mixture was then stirred at room temperature for 10 min, diluted with water (20 mL) and passed through an Oasis HLB Plus cartridge. The latter was washed with water (10 mL), dried with air (20 mL) and the radioactivity was recovered from the cartridge using absolute ethanol (2 mL) and air (3 mL). After evaporation under vacuum, the final product was formulated in saline (0.9% NaCl, 0.5 mL). Total synthesis time: 61 min. Radiochemical yield decay-corrected: 57%. The radiochemical purity of the radiotracer was verified using analytical radio RP-HPLC measurements at the end of radiosynthesis (>99%, FIG. 1).

### Biological Activity

Studies relating to in vitro binding affinity and in vivo biodistribution were conducted.

In Vivo PET imaging and Biodistribution

The human glioblastoma U87-MG cell line was purchased from ATCC. Cells were cultured in EMEM (supplemented with 2 mM L-glutamine, with 10% fetal bovine serum and 0.1 mM NEAA) at 37° C. in a humidified atmosphere (5% CO2, 95% air).

All animal experiments were conducted in accordance with the Federation for Laboratory Animal Science Associations guidelines. Healthy female Swiss nude (CrI:NU(Ico)-Foxn1nu) mice (5-6 weeks old) were purchased from Charles River. Mice were irradiated 24-72 hours prior to tumour cell inoculation (whole-body irradiation, 2 Gy/mouse), and U87-MG cells (1×10⁷ in 200 μL of RPMI 1640) were then injected subcutaneously into the right shoulder flank.

Female Swiss nude (CrI:NU(Ico)-Foxn1nu) mice each bearing a subcutaneous U87-MG tumour in the right flank (n=4) were administered with radiolabelled compound of Example 11 ([¹⁸F]—Ga—(S,R,R)—SiFA-FAP-1) (8±1.5 MBq, 100 μL per mouse) intravenously into the tail vein (approximately 3 weeks post inoculation of U87-MG cells). Static PET imaging was performed 60 minutes post-radiotracer administration under anaesthesia (2% isoflurane) using a small animal PET scanner (eXploreVISTA, GE) (n=3). Whole-body PET acquisitions were performed over two bed positions for 20 minutes. Regions of interest were generated, and radiotracer uptake in tissues was calculated as % injected dose per region of interest volume (% ID/cm³). Immediately post-PET imaging, mice (n=4) were sacrificed and select tissues (blood, tumor, heart, lung, liver, kidneys, spleen, pancreas, intestine, bone, muscle, tail) were harvested and weighed. Radioactivity in the collected samples was determined using γ-counting (Packard). Radiotracer uptake was calculated as % injected dose per gram of tissue (% ID/g).

### Results

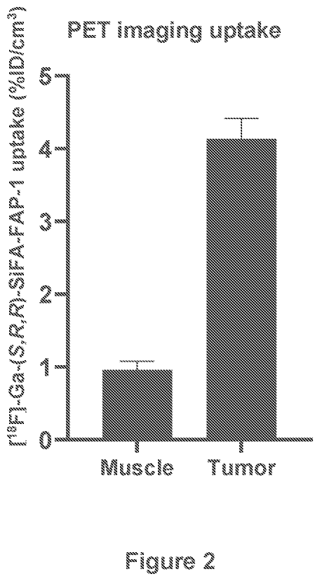

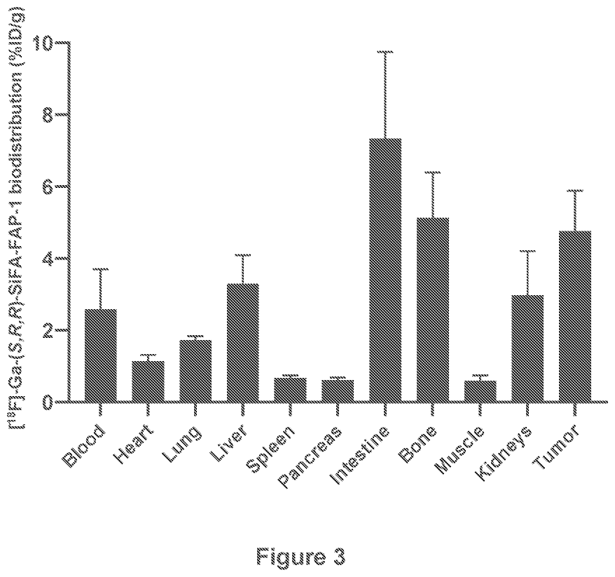

Tumor uptake of the radiolabelled compound of Example 11 ([¹⁸F]—Ga—(S,R,R)—SiFA-FAP-1) was observed using both PET imaging (Table 2, FIG. 2) and biodistribution analyses by γ-counting excised tissues (Table 3a and 3b, FIG. 3). PET imaging demonstrated radiotracer uptake greater than or equal to 4-fold higher in tumor compared to muscle (Table 2, FIG. 2). Biodistribution analyses demonstrated radiotracer uptake 8-fold higher in tumor compared to muscle (Table 3a and 3b, FIG. 3).

TABLE 2
Example 11 ([¹⁸F]-Ga-(S,R,R)-SiFA-FAP-1) PET Imaging Uptake

| Mouse ID | | Mouse 1 | Mouse 2 | Mouse 3 | Mean ± SD |
|---|---|---|---|---|---|
| % ID/cm³ | Muscle | 1 | 0.8 | 1.1 | 1.0 ± 0.1 |
| | Tumor | 4.4 | 4.2 | 3.8 | 4.1 ± 0.3 |
| | Tumor:Muscle | 4.6 | 5 | 3.5 | 4.4 ± 0.8 |
| | ratio | | | | |

TABLE 3a
Example 11 ([¹⁸F]-Ga-(S,R,R)-SIFA-FAP-1)
Biodistribution (% ID/g)

| | Mouse 1 | Mouse 2 | Mouse 3 | Mouse 4 | Mean ± SD |
|---|---|---|---|---|---|
| Blood | 2.0 | 1.3 | 3.5 | 3.5 | 2.6 ± 1.1 |
| Heart | 1.1 | 0.9 | 1.3 | 1.2 | 1.1 ± 0.2 |
| Lung | 1.6 | 1.8 | 1.7 | 1.8 | 1.7 ± 0.1 |
| Liver | 3.8 | 2.5 | 2.7 | 4.2 | 3.3 ± 0.8 |
| Spleen | 0.7 | 0.6 | 0.7 | 0.7 | 0.7 ± 0.1 |
| Pancreas | 0.7 | 0.6 | 0.6 | 0.5 | 0.6 ± 0.1 |
| Intestine | 5.4 | 5.3 | 8.6 | 10.1 | 7.3 ± 2.4 |
| Bone | 5.7 | 6.6 | 4.0 | 4.2 | 5.1 ± 1.3 |
| Muscle | 0.5 | 0.8 | 0.5 | 0.6 | 0.6 ± 0.1 |
| Kidneys | 3.8 | 1.6 | 4.2 | 2.4 | 3.0 ± 1.2 |

TABLE 3b
Example 11 ([¹⁸F]-Ga-(S,R,R)-SiFA-FAP-1)
Biodistribution - Tumor to muscle ratio (T:M)

| Mouse ID | | Mouse 1 | Mouse 2 | Mouse 3 | Mouse 4 | Mean ± SD |
|---|---|---|---|---|---|---|
| % ID/g | Muscle | 0.5 | 0.8 | 0.5 | 0.6 | 0.6 ± 0.1 |
| | Tumor | 5.8 | 4.8 | 5.2 | 3.2 | 4.8 ± 1.1 |
| | T:M | 11.0 | 5.9 | 10.6 | 5.7 | 8.3 ± 2.9 |

### In Vitro FAP Binding Affinity

The binding affinity of non-radiolabelled compounds Example 2 (Ga—(S,R,R)—SiFA-FAP-1) and Example 1 ((S,R,R)—SiFA-FAP-1) was assessed in vitro using Grating-Coupled Interferometry (GCI) and waveRAPID kinetics assay (Kartal Ö et al. SLAS Discov. 2021 September; 26(8)-995-1003).

FAP was immobilized on a streptavidin sensor chap in order to assess target binding of compounds. Briefly, the streptavidin chip was conditioned with borate buffer and activated with EDC-NHS solution. Immobilization was achieved via amine coupling to the surface on an amine reactive sensor chip (NeutrAvidin sensor chip) and unused amine reactive groups were deactivated with ethanolamine. FAP was immobilized via via NeutrAvidin capturing of the biotinylated FAP and remaining NeutrAvidin quenched with biocytin. Binding of the compounds to immobilized FAP was assessed using the waveRAPID kinetics assay, using different concentrations of each compound in 1×PBS pH 7.4, 1 mM DTT, 0.005% Tween, 2% DMSO. Binding affinity to FAP was reported as K_{D}.

### Results

The binding affinity for both compounds was in the pM range (Table 4).

TABLE 4
Binding affinity

| Binding | Example 2 | Example 1 |
|---|---|---|
| affinity | (Ga-(S,R,A)-SiFA-FAP-1) | ((S,R,A)-SIFA-FAP-1) |
| K_{D} (pM) | 44 | 31 |

BRIEF DESCRIPTION OF THE FIGURES

FIG. 1. Quality control of Example 11 ([¹⁸F]—Ga—(S,R,R)—SiFA-FAP-1) at the end of radiosynthesis.

FIG. 2. Example 11 ([¹⁸F]—Ga—(S,R,R)—SiFA-FAP-1) PET imaging uptake of U87-MG tumor-bearing mice (Table 2). Mice were injected with [¹⁸F]—Ga—(S,R,R)—SiFA-FAP-1 (8±1.5 MBq) and imaged 60 minutes post radiotracer administration. Average radiotracer uptake in muscle and tumor (% ID/cm³), mean±SD is shown (n=3). Radiotracer uptake was >4-fold higher in tumor compared to muscle.

FIG. 3. Biodistribution of Example 11 ([¹⁸F]−Ga—(S,R,R)—SiFA-FAP-1) in U87-MG tumor-bearing mice (Tables 3a and 3b). Mice were injected with Example 11 ([¹⁸F]—Ga—(S,R,R)—SiFA-FAP-1) (8±1.5 MBq). Radioactivity in tissues was measured by γ-counting 80 minutes post-radiotracer injection. Radiotracer biodistribution (% ID/g), mean±SD is shown (n=4). Biodistribution analyses demonstrated radiotracer uptake 8-fold higher in tumor compared to muscle.

1. A ligand-SIFA conjugate, comprising, within in a single molecule two separate moieties:
(a) one or more ligands which are capable of binding to Fibroblast Activation Protein (FAP), and
(b) a silicon-fluoride acceptor (SIFA) moiety which comprises a covalent bond between a silicon and a fluorine atom and which SIFA is optionally labelled with ¹⁸F;
or a pharmaceutically or diagnostically acceptable salt or solvate thereof.

2. The conjugate according to claim 1, further comprising:
(c) one or more chelating moieties, optionally containing a chelated nonradioactive or radioactive cation.

3. The conjugate according to claim 1 or 2, wherein the one or more ligands which are capable of binding to Fibroblast Activation Protein (FAP) each independently comprise one or more heterocyclic group(s), selected from optionally substituted pyrrolidinyl, quinolinyl, isoquinolinyl, quinoxalinyl, phthalazinyl, quinazolinyl, cinnolinyl and naphthyridinyl.

4. The conjugate according to claim 2, which is a conjugate of formula (4), (4a) or (4b): 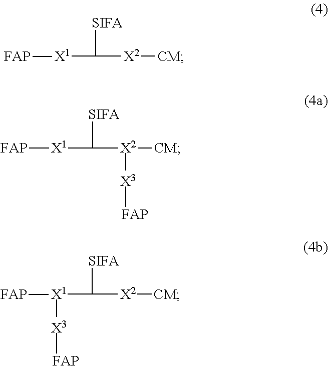
or a salt thereof, wherein X¹, X² and X³ represent divalent linking groups, and where X¹, X² and X³ together with the groups to which they are attached comprise one or more amide bonds;
FAP represents a ligand which is capable of binding to Fibroblast activation protein (FAP);
L is an optionally substituted linker group;
SIFA represents the silicon-fluoride acceptor (SIFA) moiety which comprises a covalent bond between a silicon and a fluorine atom; and
CM represents a chelating moiety, optionally containing a chelated nonradioactive or radioactive cation.

5. The conjugate according to claim 4, wherein X¹ is an optionally substituted 10-20 atom linker comprising 1 or more amide bonds, wherein the optional substituent is selected from —X³-FAP, CO₂H and CH₂OH;
X² is an optionally substituted 1-5 atom linker comprising 1 or more amide bonds, where in compounds of formula (4) or (4b), X² may also be —NH— or represent a bond;
X³ is an optionally substituted 10-20 atom linker comprising 1 or more amide bonds, wherein the optional substituent is selected from CO₂H and CH₂OH.

6. The conjugate according to any one of claims 1 to 5, wherein the silicon-fluoride acceptor (SIFA) moiety comprises the structure represented by formula (3): 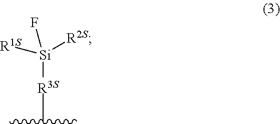
wherein R^{1S} and R^{2S} are independently a linear, branched or cyclic C₃ to C₁₀ alkyl group;
R^{3S} is a C₁ to C₂₀ hydrocarbon group comprising one or more aromatic and/or aliphatic units and/or up to 3 heteroatoms selected from O and S;
and wherein the SIFA moiety is attached to the remainder of the conjugate via the bond marked by [symbol] .

7. The conjugate according to claim 6, wherein the silicon-fluoride acceptor (SIFA) moiety comprises the structure represented by formula (3a): 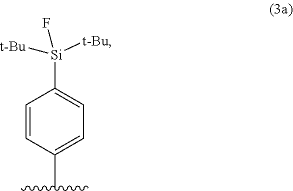 [SMILES: *c1ccc([Si](F)(C(C)(C)C)C(C)(C)C)cc1]
wherein t-Bu indicates a tert-butyl group.

8. The conjugate according to any one of claims 1 to 7, wherein the chelating moiety comprises at least one of:
(i) a macrocyclic ring structure with 8 to 20 ring atoms of which 2 or more are heteroatoms selected from oxygen atoms and nitrogen atoms;
(ii) an acyclic, open chain chelating structure with 8 to 20 main chain atoms of which 2 or more are heteroatoms selected from oxygen atoms and nitrogen atoms; or
(iii) a branched chelating structure containing a quaternary carbon atom.

9. The conjugate according to claim 8, wherein the chelating moiety is selected from bis(carboxymethyl)-1,4,8,11-tetraazabicyclo[6.6.2]hexadecane (CBTE2a), cyclohexyl-1,2-diaminetetraacetic acid (CDTA), 4-(1,4,8,11-tetraazacyclotetradec-1-yl)-methylbenzoic acid (CPTA), N′-[5-[acetyl(hydroxy)amino]pentyl]-N-[5-[[4-[5-aminopentyl-(hydroxy)amino]-4-oxobutanoyl]amino]pentyl]-N-hydroxybutandiamide (DFO), 4,11-bis(carboxymethyl)-1,4,8,11-tetraazabicyclo[6.6.2]hexadecane (DO2A) 1,4,7,10-tetracyclododecan-N,N′,N″,N′″-tetraacetic acid (DOTA), α-(2-carboxyethyl)-1,4,7,10-tetraazacyclododecane-1,4,7,10-tetraacetic acid (DOTAGA), 1,4,7,10 tetraazacyclododecane N, N′, N″, N′″ 1,4,7,10-tetra(methylene) phosphonic acid (DOTMP), N,N′-dipyridoxylethylendiamine-N,N′-diacetate-5,5′-bis(phosphate) (DPDP), diethylene triamine N,N′,N″ penta(methylene) phosphonic acid (DTMP), diethylenetriaminepentaacetic acid (DTPA), ethylenediamine-N,N′-tetraacetic acid (EDTA), ethyleneglycol-O,O-bis(2-aminoethyl)-N,N,N′,N′-tetraacetic acid (EGTA), N,N-bis(hydroxybenzyl)-ethylenediamine-N,N′-diacetic acid (HBED), hydroxyethyldiaminetriacetic acid (HEDTA), 1-(p-nitrobenzyl)-1,4,7,10-tetraazacyclodecan-4,7,10-triacetate (HP-DOA3), 6-hydrazinyl-N-methylpyridine-3-carboxamide (HYNIC), tetra 3-hydroxy-N-methyl-2-pyridinone chelators (4-((4-(3-(bis(2-(3-hydroxy-1-methyl-2-oxo-1,2-dihydropyridine-4-carboxamido)ethyl)amino)-2-((bis(2-(3-hydroxy-1-methyl-2-oxo-1,2-dihydropyridine-4-carboxamido)ethyl)amino)methyl)propyl)phenyl)amino)-4-oxobutanoic acid), abbreviated as Me-3,2-HOPO, 1,4,7-triazacyclononan-1-succinic acid-4,7-diacetic acid (NODASA), 1-(1-carboxy-3-carboxypropyl)-4,7-(carbooxy)-1,4,7-triazacyclononane (NODAGA), 1,4,7-triazacyclononanetriacetic acid (NOTA), 4,11-bis(carboxymethyl)-1,4,8,11-tetraazabicyclo[6.6.2]hexadecane (TE2A), 1,4,8,11-tetraazacyclododecane-1,4,8,11-tetraacetic acid (TETA), tris(hydroxypyridinone) (THP), terpyridin-bis(methyleneamintetraacetic acid (TMT), 1,4,7-triazacyclononane-1,4,7-tris[methylene(2-carboxyethyl)phosphinic acid] (TRAP), 1,4,7,10-tetraazacyclotridecan-N,N′,N″,N′″-tetraacetic acid (TRITA), 3-[[4,7-bis[[2-carboxyethyl(hydroxy)phosphoryl]methyl]-1,4,7-triazonan-1-yl]methyl-hydroxy-phosphoryl]propanoic acid, and triethylenetetraaminehexaacetic acid (TTHA).

10. The conjugate according to claim 9, wherein the chelating moiety is 1,4,7,10-tetracyclododecan-N,N′,N″,N′″-tetraacetic acid (DOTA), α-(2-carboxyethyl)-1,4,7,10-tetraazacyclododecane-1,4,7,10-tetraacetic acid (DOTAGA) or 1,4,7-triazacyclononane-1,4,7-tris[methylene(2-carboxyethyl)phosphinic acid] (TRAP).

11. The conjugate according to claim 9 or claim 10, wherein the chelating moiety contains a chelated cation selected from the cations of ⁴³Sc, ⁴⁴Sc, ⁴⁷Sc, ⁶¹Cu, ⁶⁴Cu, ⁶⁷Cu, ⁶⁷Ga, ⁶⁸Ga, ⁹⁰Y, ¹¹¹In, ¹⁴⁹Tb, ¹⁵²Tb, ¹⁵⁵Tb, ¹⁶¹Tb, ¹⁶⁶Ho, ¹⁷⁷Lu, ¹⁸⁶Re, ¹⁸⁸Re, ²¹²Pb, ²¹²Bi, ²¹³Bi, ²²⁵Ac, and ²²⁷Th or a cationic molecule comprising ¹⁸F.

12. The conjugate according to any one of claims 1 to 11, wherein the SIFA fluorine atom is ¹⁸F.

13. The conjugate according to any one of claims 1 to 12, wherein the FAP binding moiety comprises a substituted pyrrolidine ring.

14. The conjugate according to any one of claims 1 to 13, wherein the FAP binding moiety comprises a moiety of formula (2): 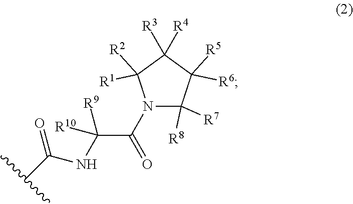
wherein, R¹, R², R³, R⁴, R⁵, R⁶, R⁷ and R⁸ are independently selected from H, OH, B(OH)₂, CO₂H, CN, halo, C₁₋₆ alkyl and —O—C₁₋₆ alkyl; and
R⁹ and R¹⁰ are independently H or C₁₋₆ alkyl.

15. The conjugate according to claim 13, comprising a moiety of formula (2a): 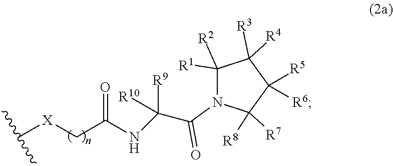
wherein, R¹, R², R³, R⁴, R⁵, R⁶, R⁷ and R⁸ are independently selected from H, OH, B(OH)₂, CO₂H, CN, halo, C₁₋₆ alkyl and —O—C₁₋₆ alkyl;
R⁹ and R¹⁰ are independently H or C₁₋₆ alkyl;
n is 0to 3; and
X is a 5 to 10-membered N-containing monocyclic or bicyclic heterocycle which optionally further comprises 1, 2 or 3 heteroatoms selected from O, N and S and is optionally substituted with 1 to 3 substituents selected from C₁₋₆ alkyl, —O—C₁₋₆ alkyl, —S—C₁₋₆ alkyl and —NR²⁰R²¹, where R²⁰ and R²¹ are independently selected from H and C₁₋₆ alkyl.

16. The conjugate according to claim 15, wherein n is 0.

17. The conjugate according to claim 15 or 16, wherein X is selected from:

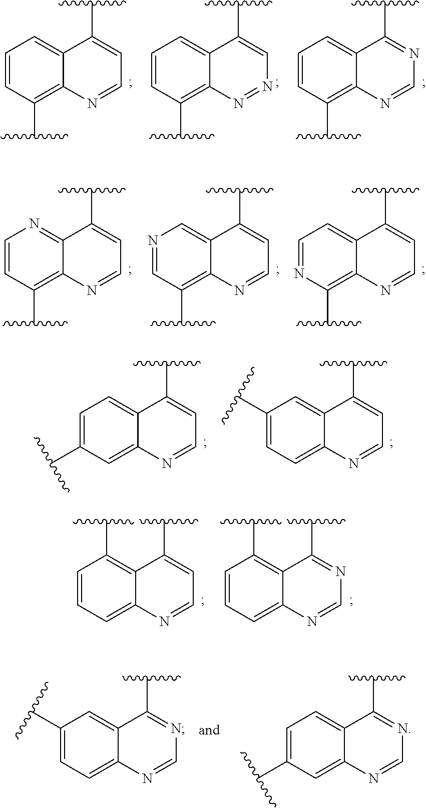

18. The conjugate according to any one of claims 14 to 17, wherein:
R³ and R⁴ are F;
R⁷ is CN; and
R¹, R², R⁵, R⁶, R⁸, R⁹ and R¹⁰ are H.

19. The conjugate according to any one of claims 1 to 12, wherein the FAP binding moiety comprises a moiety which is selected from the group consisting of: 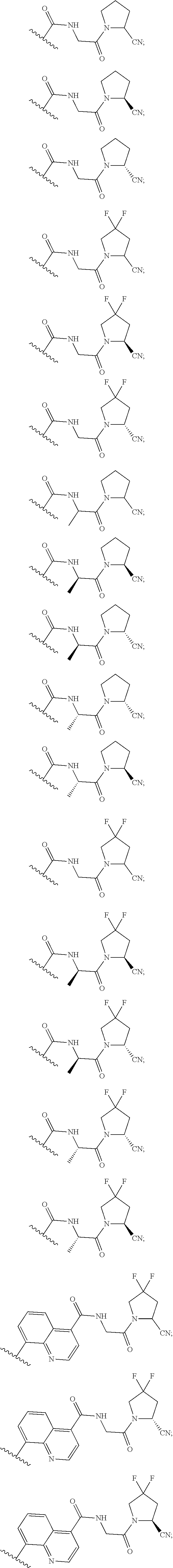 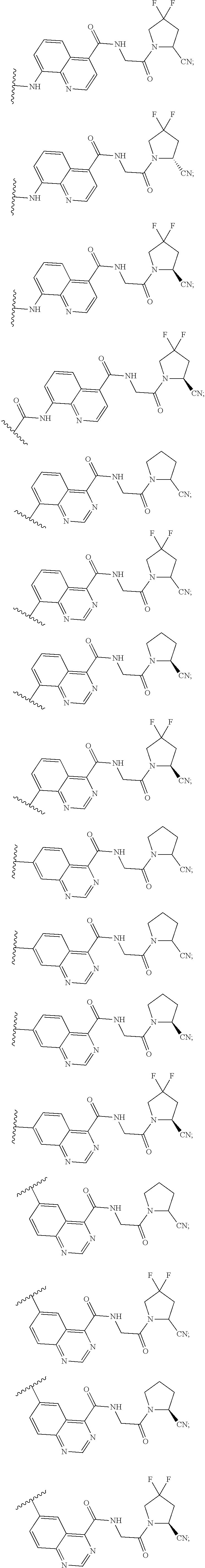 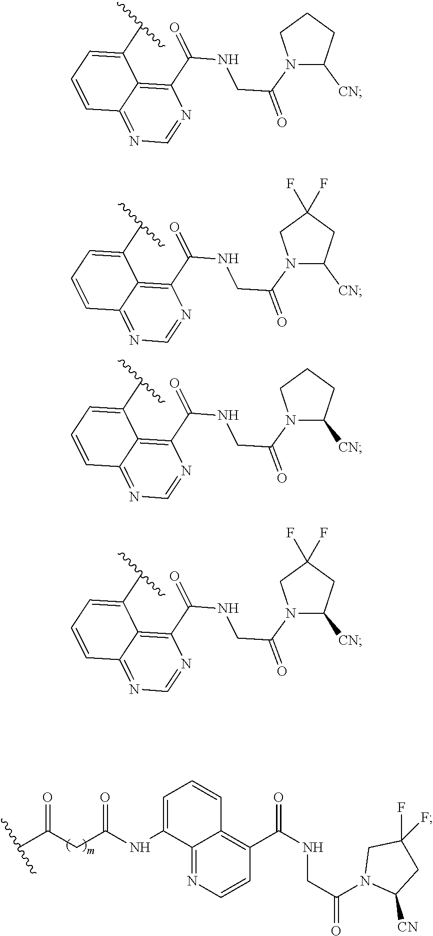
where m is 0to 10.

20. The conjugate according to any one of claims 1 to 12, wherein the FAP binding moiety comprises a cyclic peptide.

21. The conjugate according to any one of claims 1 to 12, wherein the FAP binding moiety comprises a moiety selected from the group consisting of:

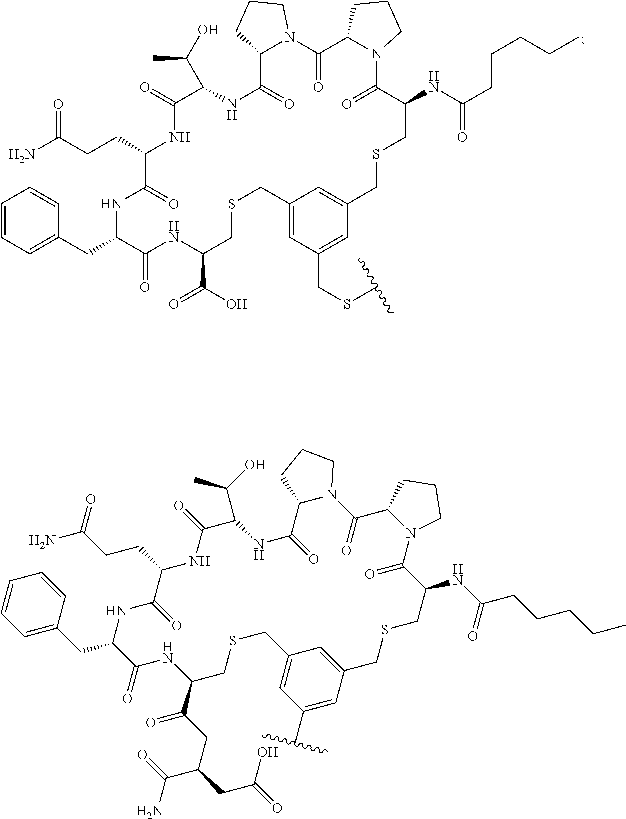

22. The conjugate according to claim 1, which is selected from the group consisting of:

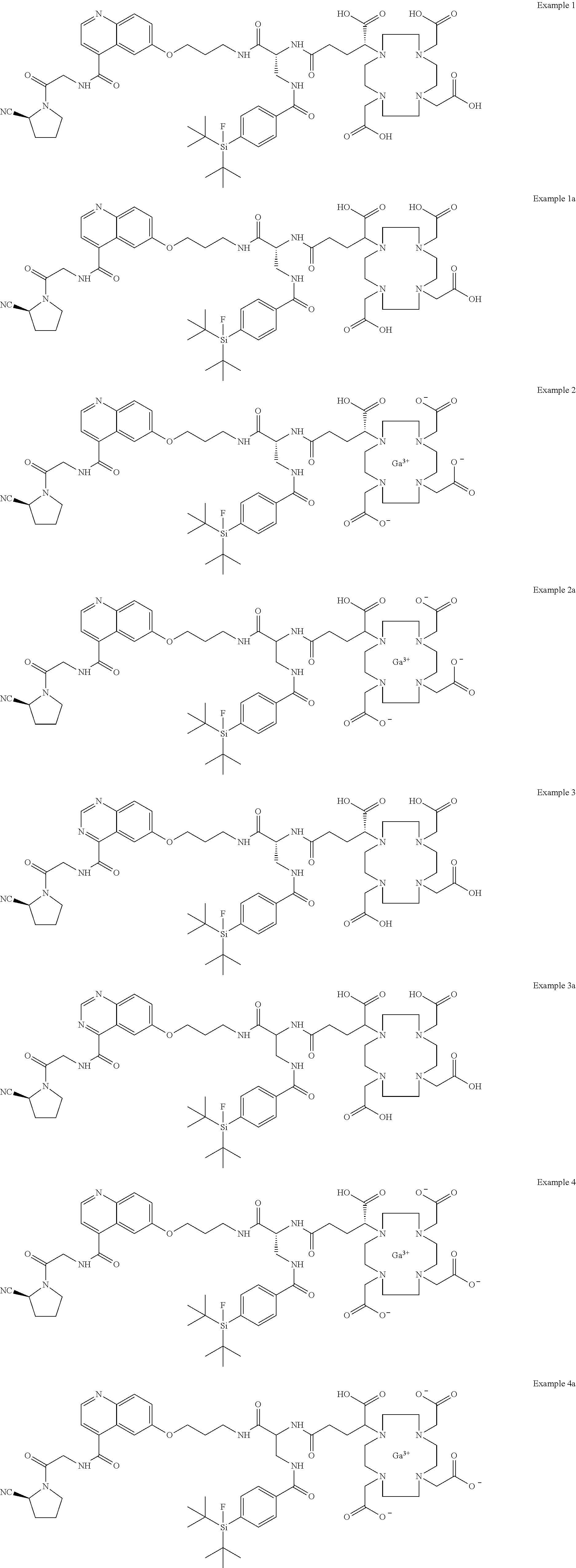

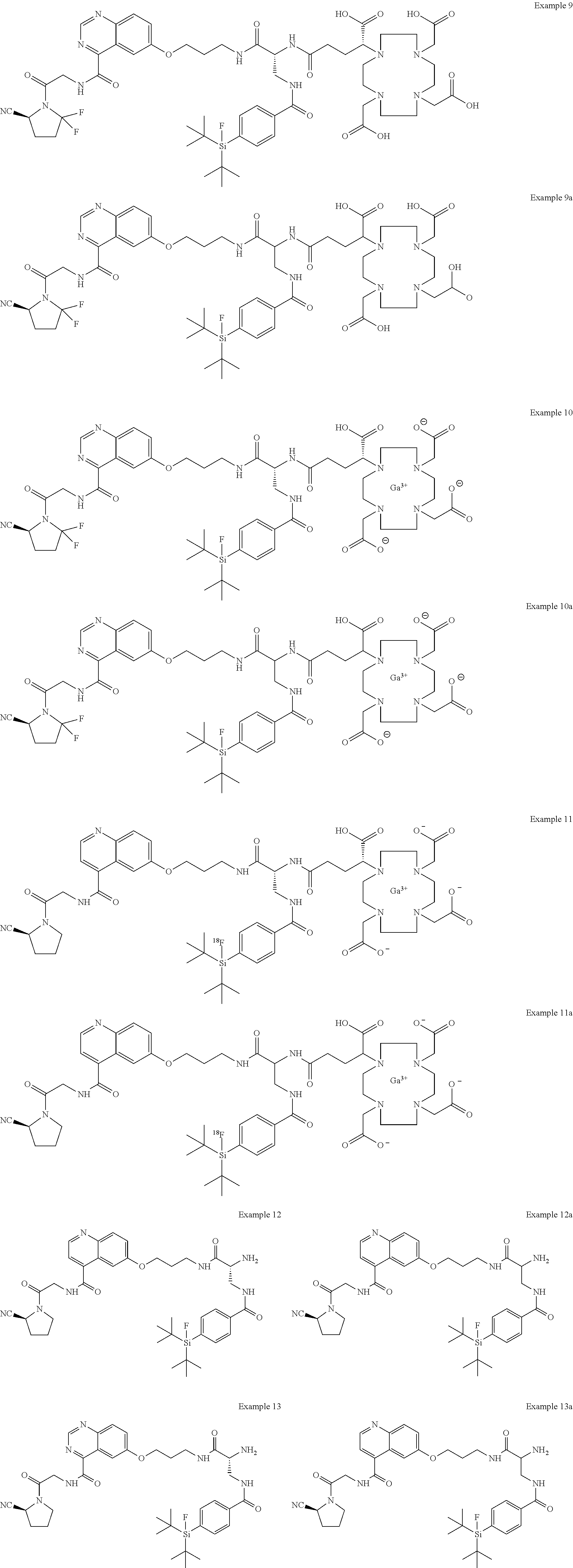

23. A pharmaceutical or diagnostic composition comprising or consisting of one or more conjugates or compounds according to any one of claims 1 to 22.

24. A conjugate, compound or composition according to any one of claims 1 to 23 for use in medicine.

25. A conjugate, compound or composition according to any one of claims 1 to 24 for use as a cancer diagnostic or imaging agent.

26. A method of imaging and/or diagnosing cancer comprising administering a conjugate, compound or composition according to any one of claims 1 to 25 to a patient in need thereof.

27. A conjugate, compound or composition according to any one of claims 1 to 24 for use in the treatment of cancer.

28. A conjugate, compound or composition according to any one of claims 1 to 25 for use in the diagnosis or treatment of cancer, chronic inflammation, atherosclerosis, fibrosis, tissue remodelling and keloid disorder.

29. The conjugate, compound or composition for use according to claim 28, wherein the cancer is selected from the group consisting of breast cancer, pancreatic cancer, small intestine cancer, colon cancer, rectal cancer, lung cancer, head and neck cancer, ovarian cancer, hepatocellular carcinoma, esophageal cancer, hypopharynx cancer, nasopharynx cancer, larynx cancer, myeloma cells, bladder cancer, cholangiocellular carcinoma, clear cell renal carcinoma, neuroendocrine tumor, oncogenic osteomalacia, sarcoma, CUP (carcinoma of unknown primary), thymus carcinoma, desmoid tumors, glioma, astrocytoma, cervix carcinoma and prostate cancer.
